(19) United States
(12) Patent Application Publication
(10) Pub. No.: US 2026/0112924 A1
(43) Pub. Date: Apr. 23, 2026

(54) SYSTEMS, DEVICES, AND METHODS FOR ESTABLISHING A WIRELESS LINK

(71) Applicant: uLink Labs, Inc., San Mateo, CA (US)

(72) Inventors: Jayant CHARTHAD, Menlo Park, CA (US); Marcus WEBER, San Francisco, CA (US)

(21) Appl. No.: 19/282,108

(22) Filed: Jul. 28, 2025

Related U.S. Application Data
(63) Continuation of application No. 17/571,817, filed on Jan. 10, 2022.
Application No. 17/571,817 is a continuation of application No. PCT/US2020/041696, filed on Jul. 10, 2020.
(60) Provisional application No. 62/872,256, filed on Jul. 10, 2019.
(60) Provisional application No. 62/929,684, filed on Nov. 1, 2019.
(60) Provisional application No. 63/036,298, filed on Jun. 8, 2020.

(51) Int. Cl. H02J 50/80; A61B 5/00; H02J 50/90; H04B 11/00; H04B 13/00; H04B 17/318

(52) U.S. Cl. CPC H02J 50/80; H02J 50/90; H04B 11/00; H04B 13/005; H04B 17/318; A61B 5/0031; A61B 5/686

(57) ABSTRACT

Described here are systems, devices, and methods for establishing a wireless link such as for exchanging wireless power, data, or signals through tissue. In some variations, a system may comprise a first device configured to generate a wireless signal. A second device may comprise a processor and one or more transducer arrays configured to receive the wireless signal from the first device. The processor may be configured to generate first device data based on the received wireless signal. The second transducer array may be configured to exchange one or more wireless signals with the first device based on the first device data.

### CROSS-REFERENCE TO RELATED APPLICATIONS

This application is a continuation of U.S. application Ser. No. 17/571,817, filed Jan. 10, 2022, which is a continuation of International Patent Application No. PCT/US2020/041696, filed Jul. 10, 2020, which claims priority to U.S. Provisional Application No. 62/872,256, filed Jul. 10, 2019, U.S. Provisional Application No. 62/929,684, filed Nov. 1, 2019, and U.S. Provisional Application No. 63/036,298, filed Jun. 8, 2020, each of which is hereby incorporated by reference in its entirety.

### TECHNICAL FIELD

Devices, systems, and methods herein relate to exchanging one or more of wireless power and data between wireless devices such as an external wireless device and an internal device disposed within a patient.

### BACKGROUND

Devices such as physiological sensors and stimulators (e.g., pacemakers) may be disposed within a body of a patient and may be configured to monitor, diagnose, and treat a patient. These conventional devices may form a wireless power and/or data link with another device disposed external to the body. However, establishing and maintaining a robust and reliable link between the external and internal device may be challenging due to tissue interference and movement of the internal device within the body during use. For example, patient breathing, movement, and heart activity may change the location and/or orientation of the internal device within the body so as to reduce the efficiency of the wireless link between the internal device and the external device. As such, additional devices, systems, and methods for establishing a wireless link may be desirable.

### SUMMARY

Described herein are systems, devices, and methods for establishing a wireless link, such as for exchanging power or data through tissue. Generally, a system may be configured to establish a closed-loop link to exchange one or more of wireless power and data. In some variations, a system may comprise a first device (e.g., an implantable medical device) configured to generate a wireless signal, and a second device (e.g., an external wireless device) comprising a first transducer array, a second transducer array, and a processor, wherein the first transducer array may be configured to receive the wireless signal from the first device, the processor may be configured to generate first device data based on the received wireless signal, and the second transducer array may be configured to exchange one or more of wireless power and data with the first device based on the first device data.

In some variations, the first device may comprise an implantable medical device and the second device may be configured to be disposed external to a body of a patient. In some variations, the first transducer array and the second transducer array may each comprise an ultrasound transducer array. In some variations, the second transducer array may comprise a one-dimensional linear array or a two-dimensional array. In some variations, the first transducer array may comprise at least three non-collinear transducer elements.

In some variations, the first transducer array and the second transducer array may comprise distinct transducer elements. In some variations, the first transducer array and the second transducer array may comprise at least one same transducer element. In some variations, the first transducer array may comprise a subset of the second transducer array. In some variations, the second device may comprise a third transducer array configured to transmit an interrogation signal to the first device, and the wireless signal may comprise a feedback signal generated in response to the interrogation signal. In some variations, the third transducer array may comprise distinct transducer elements from each of the first transducer array and the second transducer array. In some variations, one or more transducer elements of the second transducer array may be configured to receive the wireless signal from the first device. In some variations, the wireless signal may comprise wireless data.

In some variations, one or more transducer elements of the first transducer array and the second transducer array may be interleaved or interspersed. In some variations, the second transducer array may be configured to exchange one or more of the wireless power and data with the first device based at least in part on one or more of an interpolation and extrapolation of the wireless signal.

Also described are systems configured to establish a closed-loop link to exchange one or more of wireless power and data. In some variations, a system may comprise a first device, and a second device comprising a processor and a transducer array comprising a plurality of sub-arrays, wherein a first sub-array may be configured to transmit an interrogation signal to the first device, a second sub-array may be configured to receive a feedback signal from the first device, and wherein the processor may be configured to cycle through one or more sub-arrays of the plurality of sub-arrays until the received feedback signal satisfies a predetermined condition.

In some variations, the first device may comprise an implantable medical device, and the second device may be configured to be disposed external to a body of a patient. In some variations, the transducer array may comprise an ultrasound transducer array. In some variations, the sub-array may comprise one or more transducer elements of the transducer array. In some variations, the first sub-array and the second sub-array may comprise the same transducer elements. In some variations, the predetermined condition may comprise a strength of the received feedback signal calculated for one or more transducer elements of the second sub-array. In some variations, the processor may be configured to select a transducer configuration based on the received feedback signal that may satisfy the predetermined condition, the transducer configuration configured to exchange one or more of the wireless power and data with the first device. In some variations, the transducer configuration may comprise one or more transducer elements of the transducer array.

Also described are systems configured to establish a closed-loop link to exchange one or more of wireless power and data. In some variations, a system may comprise a first device, and a second device comprising a processor and a transducer array comprising a plurality of sub-arrays, wherein a first sub-array may be configured to transmit an interrogation signal to the first device, and a second sub-array may be configured to receive a feedback signal from the first device, the feedback signal comprising one or more of digital first device energy data and digital interrogation signal strength data, wherein the processor may be configured to select a transducer configuration based on the feedback signal, the transducer configuration configured to exchange one or more of wireless power and data with the first device.

In some variations, the first device may comprise an implantable medical device and the second device may be configured to be disposed external to a body of a patient. In some variations, the transducer array may comprise an ultrasound transducer array. In some variations, the sub-array may comprise one or more transducer elements of the transducer array. In some variations, the first sub-array and the second sub-array may comprise the same transducer elements. In some variations, the transducer configuration may comprise one or more transducer elements of the transducer array. In some variations, the first device may comprise a power source comprising one or more of a rechargeable battery, capacitor, supercapacitor, and non-rechargeable battery. In some variations, the digital first device energy data may comprise a power source parameter comprising one or more of a voltage, energy level, charging voltage, and charging current. In some variations, the transducer configuration may be configured to wirelessly recharge the power source.

In some variations, the interrogation signal may comprise a first frequency and the one or more of the wireless power and data may comprise a second frequency different than the first frequency. In some variations, the first device may comprise at least one ultrasound transducer comprising a first impedance corresponding to the first frequency and a second impedance corresponding to the second frequency, the first impedance greater than the second impedance. In some variations, the first device may comprise a first ultrasound transducer comprising a first impedance corresponding to the first frequency, and a second ultrasound transducer comprising a second impedance corresponding to the second frequency, the first impedance greater than the second impedance.

In some variations, the interrogation signal may comprise a broad ultrasound beam. In some variations, the first device may comprise an ultrasound transducer, and a diameter of the broad ultrasound beam upon emission from the first device may comprise a diameter greater than a dimension of the ultrasound transducer. In some variations, the interrogation signal may comprise one or more of an identifier, code, and command. In some variations, the interrogation signal may comprise a radio-frequency (RF) signal.

In some variations, the feedback signal may comprise one or more analog pulses. In some variations, the feedback signal may comprise one or more of an analog pulse, acknowledgment signal, a digital first device energy state, digital interrogation signal strength, identification number, code, command, one or more parameters of the first device, the wireless power signal, and the data signal. In some variations, the feedback signal may comprise one or more ultrasonic reflection signals corresponding to the interrogation signal. In some variations, the feedback signal may comprise one or more ultrasonic backscatter signals corresponding to the interrogation signal. In some variations, the first device may be configured to modulate the ultrasonic backscatter signal. In some variations, the first device may be configured to transmit the feedback signal at one or more frequencies. In some variations, the processor may be configured to identify a frequency of the transducer configuration for transmitting one or more of wireless power and downlink data to the first device based on the feedback signal. In some variations, the identified frequency of the transducer configuration may correspond to the feedback signal frequency at a maximum amplitude. In some variations, the feedback signal may comprise periodic transmission of one or more of analog and digital feedback signals.

In some variations, the transducer configuration may comprise one or more transducer elements configured to focus one or more of the wireless power and data to the first device. In some variations, the transducer configuration may comprise one or more transducer elements configured to beamform signals. In some variations, the transducer configuration may be configured to deactivate a set of transducer elements of the transducer array based on a strength of the received feedback signal.

In some variations, the transducer configuration may be selected based on one or more of time reversal, triangulation and estimating a strength of the feedback signal. In some variations, the transducer configuration may be selected based on one or more of time reversal, triangulation and estimating a strength of the one or more analog pulses. In some variations, the processor may be configured to adjust one or more of transmit power and transmit duration of the transducer configuration based on the feedback signal. In some variations, the processor may be configured to monitor one or more of a time-averaged output power of the second device, a peak output power of the second device, heating of one or more of the second device and skin, heating of the first device, heating of a tissue structure, acoustic intensity in tissue, and an energy level of the second device. In some variations, the first device may be configured to monitor one or more of a heating of the first device and an acoustic intensity incident on the first device. In some variations, the processor may be configured to adjust one or more of transmit power and transmit duration of the transducer configuration based on one or more of a time-averaged output power of the second device, a peak output power of the second device, heating of one or more of the second device and skin, heating of the first device, heating of a tissue structure, acoustic intensity in tissue, and an energy level of the second device. In some variations, the processor may be configured to localize the first device, and adjust one or more of transmit power and transmit duration of the transducer configuration based on the feedback signal. In some variations, the processor may be configured to localize the first device based on the one or more analog pulses, and adjust one or more of transmit power and transmit duration of the transducer configuration based on one or more of digital first device energy data and digital interrogation signal strength data.

Also described are methods of establishing a close-loop link to exchange one or more wireless signals. In some variations, a method may comprise the steps of transmitting an interrogation signal to a first device using a first sub-array of a second device, receiving a feedback signal from the first device using a second sub-array of the second device, selecting one or more transducer configurations of the second device based on the feedback signal, and exchanging one or more wireless signals with the first device using the one or more transducer configurations of the second device during a plurality of intervals, wherein the wireless signals comprise one or more of a power signal, data signal, interrogation signal, feedback signal, downlink signal and uplink signal.

In some variations, the method may further comprise transmitting the feedback signal from the first device in response to one or more wireless signals received by the first device during one or more of the plurality of intervals. In some variations, the method may further comprise detecting one or more of a falling edge of one or more wireless signals and a code corresponding to one or more wireless signals received by the first device.

In some variations, selecting one or more transducer configurations of the second device may comprise one or more of determining one or more of a frequency, a delay, a phase, an amplitude and a gain of the selected one or more transducer elements based at least in part on one or more of a delay, phase, arrival time, time of flight, amplitude, frequency, and encoded data of the feedback signal.

In some variations, the method may further comprise determining to transmit one or more of a power signal, interrogation signal, data signal and a downlink signal to the first device in response to the received feedback signal. In some variations, the method may further comprise determining to inhibit transmission of the wireless signal to the first device in response to the received feedback signal. In some variations, a transducer configuration corresponding to a subsequent interval may be selected based on one or more previously received feedback signals during one or more previous intervals. In some variations, a duration of at least one interval of the plurality of intervals may be determined by the first device. In some variations, a duration of at least one interval of the plurality of intervals may be determined by the second device. In some variations, the first device may be configured to periodically transmit the feedback signals during one or more of the intervals.

In some variations, the one or more transducer configurations may be selected based on time reversal. In some variations, the method may further comprise identifying a frequency of the feedback signal, wherein the one or more transducer configurations may comprise the identified frequency. In some variations, the method may further comprise identifying a frequency of the feedback signal, wherein the one or more transducer configurations may comprise a frequency different from the identified frequency.

In some variations, selecting one or more transducer configurations of the second device may comprise estimating a set of spatial coordinates of the first device using triangulation, wherein exchanging one or more of the wireless power signal and data signal using the one or more transducer configurations may be based at least in part on the estimated spatial coordinates.

In some variations, selecting one or more of the transducer configurations of the second device may comprise estimating a strength of the feedback signal received by the second sub-array of the second device, and exchanging one or more of the wireless power signal and data signal using the one or more transducer configurations based on the estimated strength of the received feedback signal.

In some variations, the feedback signal may comprise a digital amplitude of the interrogation signal received by the first device, and selecting the one or more transducer configurations of the second device may comprise selecting one or more of the sub-arrays corresponding to a maximum digital amplitude of the interrogation signal.

In some variations, the feedback signal may comprise a first feedback signal, and the method may further comprise powering the first device by transmitting a first power signal during a first power interval, and receiving a second feedback signal from the first device after the first power interval. In some variations, the method may further comprise powering the first device intermittently, wherein the second device may be configured to inhibit powering of the first device based on the feedback signal. In some variations, the interrogation signal may be a first interrogation signal, and the method may further comprise transmitting a second interrogation signal to the first device after a time delay. In some variations, the first device may be configured to transmit the feedback signal after a time delay.

In some variations, the method may further comprise selecting the transducer configuration based on a location of the first device. In some variations, the method may further comprise storing the transducer configuration corresponding to the location of the first device in a memory of the second device. In some variations, the method may further comprise selecting the stored transducer configuration for exchanging one or more of the wireless power signal and data signal with the first device.

In some variations, a duration of the interval may be predetermined. In some variations, the first device may be configured to transmit a plurality of feedback signals upon receiving the interrogation signal. In some variations, the plurality of feedback signals may comprise pulses periodically transmitted by the first device. In some variations, the method may further comprise estimating a spatial path of the first device based on the plurality of feedback signals. In some variations, the method may further comprise selecting the transducer configuration corresponding to the spatial path of the first device based on the estimated spatial path.

In some variations, the method may further comprise generating a location notification corresponding to a spatial adjustment of the second device. In some variations, generating the location notification may be based on the estimated spatial path of the first device. In some variations, the spatial adjustment may comprise aligning an axis of the second device with the spatial path of the first device. In some variations, the second device may comprise a one-dimensional linear ultrasound transducer array, and the spatial adjustment may comprise aligning one or more of an aperture and an elevation of the array with the spatial path of the first device. In some variations, the location notification may be based on a position of the transducer configuration relative to one or more of a center, edge, and predetermined location of the second device. In some variations, generating the location notification may be based on the feedback signal. In some variations, the method may comprise generating a power notification comprising a power state of one or more of the first device and the second device. In some variations, the method may comprise generating a communication notification corresponding to one or more of data received from the first device, physiological parameter data, and parameter data of one or more of the first device and the second device.

Also described are systems configured to exchange one or more of wireless power and data. In some variations, a system may comprise a first device comprising a plurality of transducers configured to receive a downlink signal, a second device configured to transmit the downlink signal, wherein one or more of the plurality of transducers may be configured to exchange one or more of wireless power and data with the second device based on the received downlink signal.

In some variations, the first device may comprise an implantable medical device, and the second device may be configured to be disposed external to a body of a patient. In some variations, the plurality of transducers may comprise a plurality of ultrasound transducers. In some variations, the system may further comprise a power circuit configured to DC combine the received power. In some variations, the downlink signal may comprise one or more of an interrogation signal, power signal, and downlink data. In some variations, one or more of the plurality of transducers of the first device may be configured to exchange wireless data with the second device at a first frequency different from a second frequency of the received wireless power.

In some variations, the first device may further comprise a processor. In some variations, the processor may be configured to select one or more of the plurality of transducers configured to exchange one or more of the wireless power and data with the second device based on the received downlink signal. In some variations, the processor may be configured to update the selection periodically based on one or more of the received downlink signals. In some variations, the processor may be configured to calculate a received signal strength of the downlink signal for one or more of the plurality of transducers and compare the received signal strengths of one or more of the plurality of transducers against each other. In some variations, the processor may be configured to select one or more of the plurality of transducers corresponding to the received signal strength above a predetermined threshold, for exchanging one or more of the wireless power and data with the second device. In some variations, the processor may be configured to select one transducer corresponding to a maximum received signal strength for transmitting an uplink signal to the second device. In some variations, the processor may be configured to decode one or more downlink commands based on the downlink signal. In some variations, the processor may be configured to select one or more transducers for exchanging one or more of the wireless power and data with the second device based on decoding one or more of the downlink commands.

Also described are systems configured to exchange one or more of wireless power and data. In some variations, a system may comprise a first device configured to transmit an interrogation signal through a transmission medium, the interrogation signal in the transmission medium configured to generate a reflected interrogation signal, and a second device configured to receive the interrogation signal from the first device and to transmit a feedback signal comprising at least one parameter different from the reflected interrogation signal.

In some variations, the first device may be configured to be disposed external to a body of a patient, and the second device may comprise an implantable medical device. In some variations, the at least one parameter may comprise one or more of an amplitude, a signal strength, phase, frequency, time delay, and signal modulation. In some variations, the second device may be configured to transmit a feedback signal using one or more of active signal transmission and backscatter modulation. In some variations, the at least one parameter may comprise a time delay, wherein the second device may be configured to transmit the feedback signal after receiving the interrogation signal and the time delay. In some variations, the time delay may be at least about 10 microseconds. In some variations, the interrogation signal may comprise a first modulation and the feedback signal may comprise a second modulation different from the first modulation. In some variations, the interrogation signal may comprise an ultrasonic signal and the feedback signal may comprise a radio-frequency signal. In some variations, the interrogation signal may comprise a radio-frequency signal and the feedback signal may comprise an ultrasonic signal. In some variations, the feedback signal may comprise one or more of a code and a waveform feature that is different from one or more of the reflected interrogation signals.

Also described are methods of positioning a wireless on the body. In some variations, the method may comprise the steps of generating a user prompt corresponding to a desired location on the body, and orienting the wireless device according to one or more of an orientation feature and an orientation signal of the wireless device.

In some variations, providing the user prompt may comprise one or more of a body location image, visual instructions, and audio instructions. In some variations, the orientation feature of the wireless device may comprise one or more of a marking, structure, and shape of the wireless device. In some variations, the orientation signal of the wireless device may comprise signals from one or more of an orientation sensor, an accelerometer, a gyroscope, and a position sensor.

In some variations, the method may comprise the steps of measuring a parameter of one or more of the wireless device and the body, estimating a position of the wireless device on the body based on the measured parameter, and generating a user prompt corresponding to the estimated position of the wireless device on the body.

In some variations, the parameter may comprise one or more of a heart sound, lung sound, breathing sound, wireless signal coming from an implantable medical device, and wireless reflection signal. In some variations, the user prompt may comprise one or more of a notification about the estimated position of the wireless device on the body, and a recommendation comprising one or more of repositioning the wireless device on the body, and contacting a health care professional. In some variations, repositioning the wireless device on the body may comprise one or more of moving, adjusting, and rotating the wireless device.

Also described are methods of coupling an ultrasonic device to a body of a patient. In some variations, the method may comprise the steps of measuring a parameter of one or more of the ultrasonic device and the body, estimating a coupling state between the ultrasonic device and the body based on the measured parameter, and generating a user prompt corresponding to the coupling state between the ultrasonic device and the body.

In some variations, the ultrasonic device may comprise one or more ultrasound transducers. In some variations, the parameter may comprise one or more of an electrical impedance of the ultrasound transducer, reflection coefficient of the ultrasound transducer, heart sound, lung sound, ultrasonic signal transmitted from an implantable medical device, ultrasonic reflection signal, pressure, force, touch, capacitance, electrical impedance of tissue, heat, and temperature.

In some variations, estimating the coupling state may comprise estimating one or more of adequacy and degree of coupling between the ultrasonic device and the body. In some variations, the user prompt may comprise one or more of the coupling state and a recommendation comprising one or more of repositioning the ultrasonic device against the body, applying an ultrasonic coupling agent, adjusting a fastener of the ultrasonic device to the body, and contacting a health care professional.

In some variations, the method may further comprise periodically transmitting uplink signals from an implantable medical device, wherein estimating the coupling state may be based on measuring a strength of one or more of the uplink signals received by the ultrasonic device. In some variations, the method may further comprise transmitting an interrogation signal from the ultrasonic device and receiving one or more feedback signals from an implantable medical device, wherein estimating the coupling state may be based on measuring a strength of one or more of the feedback signals received by the ultrasonic device.

Also described are methods of noise reduction for decoupling an ultrasound signal from a pressure signal. In some variations, a method may comprise the steps of measuring a parameter of an ultrasound signal received by one or more of an ultrasound transducer, pressure transducer, flow sensor, force sensor and MEMS device; and generating pressure data based on a pressure signal measured by the pressure transducer and the measured parameter of the ultrasound signal.

In some variations, generating the pressure data may comprise decoupling the ultrasound signal from the pressure signal. In some variations, decoupling the ultrasound signal from the pressure signal may comprise one or more of averaging, digital signal processing, and analog signal processing. In some variations, generating the pressure data may comprise identifying one or more pressure samples of the pressure data comprising the measured parameter of the ultrasound signal, and rejecting or flagging the identified one or more pressure samples.

In some variations, the method may further comprise measuring a pressure signal using the pressure transducer after a time delay. In some variations, the time delay may be predetermined. In some variations, the time delay may be determined based on dissipation of the ultrasound signal.

In some variations, generating the pressure data may be performed by a processor of a first device comprising the ultrasound transducer and the pressure transducer. In some variations, generating the pressure data may be performed by a processor of a second device in wireless communication with a first device comprising the ultrasound transducer and the pressure transducer.

Also described are methods of noise reduction for decoupling an ultrasound signal from a pressure signal. In some variations, a method may comprise the steps of receiving an ultrasound signal using a pressure transducer of a device and filtering the ultrasound signal using a filter coupled to the pressure transducer. In some variations, filtering the ultrasound signal may comprise one or more of analog filtering, digital filtering, analog post-processing, digital post-processing, and using one or more of an amplifier, processor, integrator, averager, and boxcar sampler.

Also described are methods of estimating heart rate. In some variations, a method may comprise the steps of measuring blood pressure samples using a first device, generating blood pressure data using the measured blood pressure samples, and estimating a heart rate over one or more cardiac cycles using the blood pressure data.

In some variations, estimating the heart rate may be performed by a processor of the first device. In some variations, estimating the heart rate may be performed by a processor of a second device, the second device in wireless communication with the first device. In some variations, estimating the heart rate may comprise comparing one or more of the blood pressure samples to a predetermined threshold, identifying two or more cross-over points where the blood pressure samples may cross the predetermined threshold, and estimating a heart rate based on one or more elapsed times between the identified cross-over points.

In some variations, estimating the heart rate may comprise identifying points of local maxima or minima in the blood pressure samples, and estimating a heart rate based on one or more elapsed times between two or more points of local maxima or minima. In some variations, estimating the heart rate may comprise identifying points of a maximum or minimum rate of change in the blood pressure samples, and estimating a heart rate based on one or more elapsed times between two or more points of the maximum or minimum rate of change. In some variations, estimating the heart rate may be based on a frequency domain representation of the blood pressure samples.

Also described are systems configured to exchange one or more of wireless power and data. In some variations, a system may comprise a first device configured to traverse a spatial path within a patient, and a second device configured to exchange a wireless signal with the first device only during an access period.

In some variations, the first device or the second device may comprise a sensor configured to measure one or more physiological parameters of the patient, and a processor configured to identify the access period based on the measured one or more physiological parameters. In some variations, the one or more physiological parameters may comprise one or more of blood pressure, heart rate, breathing rate, heart sound, lung sound and ECG.

Also described are methods of parameter tracking. In some variations, a method may comprise the steps of tracking one or more parameters corresponding to a wireless system comprising a first device and a second device, selecting a transducer configuration of the second device based at least in part on the parameter, and exchanging one or more wireless signals with the first device using the selected transducer configuration.

In some variations, the parameter may comprise one or more of wireless link gain between the first device and the second device, transmit power of the second device, transmit frequency of the second device, one or more parameters of the transducer configuration, one or more parameters of the first device, energy state of the first device, battery life of the first device, a parameter corresponding to a sensor of the first device, a parameter corresponding to a transducer of the first device, transmit frequency of the first device, transmit power of the first device, one or more positions of the first device, one or more orientations of the first device and a physiological parameter of a body.

### BRIEF DESCRIPTION OF THE DRAWINGS

FIG. 1 is a schematic block diagram of an illustrative variation of a wireless system.

FIG. 2 is an illustrative cross-sectional schematic view of a variation of a wireless system.

FIG. 3A is an illustrative cross-sectional schematic view of a variation of a wireless system comprising a sub-array configured to transmit an interrogation signal. FIG. 3B is an illustrative cross-sectional schematic view of a variation of a wireless system comprising a feedback signal generated from a first device. FIG. 3C is an illustrative cross-sectional schematic view of a variation of a wireless system comprising a transducer configuration configured to transmit wireless power to a first device.

FIG. 4 is an illustrative cross-sectional schematic view of a single ultrasound transducer and ultrasonic beam in tissue.

FIG. 5 is a schematic block diagram of an illustrative variation of a first device such as an IMD.

FIG. 6 is a flowchart of an illustrative variation of a method of exchanging wireless power or data with a device.

FIGS. 7A, 7B, and 7C are timing diagrams of illustrative variations of a method of interval-based powering.

FIG. 8 is a timing diagram of an illustrative variation of a method of intermittent powering.

FIG. 9 is an illustrative variation of a wireless system comprising an external wireless device comprising a plurality of arrays.

FIG. 10 is an illustrative schematic view of a spatial path of a first device (e.g., IMD) and a corresponding access period timing diagram.

FIG. 11 is a timing diagram of an illustrative variation of physiological signals used to determine an access period.

FIG. 12 is a schematic block diagram of an illustrative variation of an first device (e.g., IMD) configured to select a transducer for an operation.

FIG. 13 is a flowchart of an illustrative variation of a method of positioning a wireless device on the body.

FIG. 14 is a flowchart of another illustrative variation of a method of positioning a wireless device on the body.

FIG. 15 is a flowchart of an illustrative variation of a method of coupling an ultrasonic device to a body of a patient.

FIG. 16 is a flowchart of an illustrative variation of a method of noise reduction.

FIG. 17 is a flowchart of another illustrative variation of a method of noise reduction.

FIG. 18 is a flowchart of an illustrative variation of a method of estimating heart rate.

FIG. 19 is a timing diagram of an illustrative variation of a method of estimating heart rate from a pressure signal.

### DETAILED DESCRIPTION

### I. Systems

### A. Overview

Generally described here are systems, devices, and methods for establishing a wireless link between a set of devices including at least one device disposed within a body of a patient and a wireless device such as a device disposed external to the patient. The systems described herein may comprise one or more wireless devices (e.g., implantable medical device, ingestible device, sensor, stimulator, and the like) disposed within a body of a patient and an external device configured to be disposed on, for example, a skin of a patient. A wireless device disposed within the body may be useful for one or more of monitoring, diagnosis, and treatment of a disease such as heart failure, prosthetic valve dysfunction, valvular heart disease, restenosis, and the like. For example, monitoring of a physiological parameter (e.g., blood pressure) of a patient by the wireless device disposed in the body may be used for the diagnosis and/or monitoring of heart failure and/or other cardiovascular (CV) diseases.

In some variations, a wireless system may comprise one or more wireless devices such as an implantable medical device (IMD) and an external wireless device. In some variations, the IMD may be implanted in a patient's body for performing one or more functions such as monitoring physiological signals or parameters (e.g., blood pressure, blood flow, neural action potentials, etc.) and stimulating tissue (e.g., nerve, muscle, etc.). In some variations, the IMD may be configured to receive wireless power from another wireless device. Additionally, or alternatively, the IMD may comprise a power source (e.g., capacitor, battery, etc.) configured to be recharged by the external wireless device. In some variations, the IMD may be configured to wirelessly communicate data and/or commands bi-directionally with another wireless device. In such systems, establishing a reliable and/or efficient wireless link for power and/or data transfer may be important for minimizing energy usage (e.g., dissipation) of the IMD and the external wireless device, minimizing tissue heating, and achieving error free data transfer for accurate disease monitoring and therapy.

In some variations, a system for exchanging wireless power or data may include an external wireless device comprising a plurality of transducer arrays each configured to perform separate functions. System efficiency may be improved by decoupling the transducer arrays from each other such that their respective transducer configurations may be optimized. For example, a first transducer array of the external device may be configured to receive a wireless signal from an IMD and a processor may be configured to generate data based on the received wireless signal. A second transducer array may be configured to exchange one or more of power and data with the IMD.

In some variations, exchanging wireless power or data between an internal device (e.g., first device) and an external device (e.g., second device) may be interrupted when the internal device moves within the body or is misaligned with respect to the external device. For example, the external device may not reliably receive a feedback signal and an IMD may be inconsistently receive interrogation signals when the IMD disposed within the heart moves with the beating of the heart. In some variations of the systems, devices, and methods described herein, the sub-arrays of an external wireless device may be cycled to transmit interrogation signals one-by-one until a received feedback signal satisfies a predetermined condition.

In some variations, a feedback signal generated by an IMD may comprise one or more of digital first device energy data and digital interrogation signal data. An external device receiving the feedback signal may be configured to select a transducer configuration for wireless power and data exchange based on the feedback signal. This data in the feedback signal may allow one or more of localization of the first device, establishing an efficient wireless link with the first device and efficiently recharging a power source of the first device, thereby reducing charging time and minimizing tissue heating. In some variations, the feedback signal may further comprise one or more analog pulses. In some variations, transducer configuration may be selected based on one or more of time reversal, triangulation and an estimated strength of the feedback signal. In some variations, the processor may be configured to adjust one or more of transmit power and transmit duration of the transducer configuration based on the feedback signal.

In some variations, an IMD disposed within the body of a patient may be powered more efficiently based on an interval-based powering method or intermittent powering method. These methods may establish efficient wireless links with a moving IMD such as an IMD implanted in the heart. In some variations, the one or more transducer configurations may be selected based on time reversal. In some variations, selecting one or more transducer configurations of the second device may comprise estimating a set of spatial coordinates of the first device using triangulation.

In some variations, the efficiency and reliability of a wireless link may be improved by identifying one or more transducers of an IMD having the highest link gain with the external wireless device. For example, a system may comprise an IMD configured to receive a downlink signal from an external wireless device. One or more of the plurality of transducers of the first device may be configured to exchange one or more of wireless power and data with the second device with the second device based on the received downlink signal.

Interrogation signals transmitted through a transmission medium such as tissue may generate reflections that may interfere with other signals such as feedback signals. In some variations, a received feedback signal transmitted by an IMD may be distinguished from reflections of an interrogation signal, thereby enabling accurate localization of an IMD and subsequent establishment of a wireless link.

In some variations, a user (e.g., patient) may position an external wireless device (e.g., a handheld device, a wearable device) on their body in order to establish a wireless link to an IMD (e.g., for recovering physiological data from the IMD). The alignment between the external wireless device and an IMD may correspond to link efficiency and the ability to establish a reliable (e.g., robust) wireless link. In some variations, a method of positioning a wireless device on the body may comprise generating a user prompt corresponding to a desired location on the body and orienting the wireless device according to one or more of an orientation feature and an orientation signal of the wireless device. These and other methods may be enable at-home monitoring by instructing (e.g., guiding) users such as patients on how to position the wireless device for disease monitoring and/or therapy.

In some variations, a position of a wireless device disposed on an external surface of the body may be estimated and a user prompt may be provided to a user to manual alignment of the wireless device to an internal device (e.g., IMD). Alignment of the wireless device to the internal device may improve a wireless link established between them. For example, a method of positioning a wireless device on the body may comprise measuring a parameter of one or more of the wireless device and the body, estimating a position of the wireless device on the body based on the measured parameter, and generating a user prompt corresponding to the estimated position of the wireless device on the body.

In some variations, an ultrasonic device disposed on an external surface (e.g., skin) of a patient may be used to wirelessly power and/or communicate with an IMD using ultrasound signals. An adequate coupling may be desired between an ultrasonic device and the skin or tissue in order to efficiently exchange ultrasound signals into and out of the body. In some variations, a method of coupling an ultrasonic device to a body of a patient may comprise measuring a parameter of one or more of the ultrasonic device and the body and estimating a coupling state between the ultrasonic device and the body based on the measured parameter. A user prompt corresponding to the coupling state between the ultrasonic device and the body may be generated based on the estimated coupling state. This may enable automatic coupling state detection between an ultrasonic device and the body. A user may be instructed on how to provide adequate coupling between the ultrasonic device and the body, thereby improving patient outcomes.

In some variations, an IMD configured to measure pressure may be configured to exchange ultrasound signals such as power and/or data with an external wireless device. Ultrasound signals propagate in the form of pressure waves and may couple or interfere with pressure signals of the IMD, thereby potentially corrupting physiological pressure data. In some variations, a method of noise reduction may comprise measuring a parameter of an ultrasound signal received by one or more of an ultrasound transducer, pressure transducer, flow sensor, force sensor and micro-electromechanical (MEMS) device. Pressure data may be generated based on a pressure signal measured by the pressure transducer and the measured parameter of the ultrasound signal. In some variations, the ultrasound signal may be decoupled from the pressure signal, thereby allowing accurate recovery of physiological pressure data.

In some variations, a method of noise reduction may comprise receiving an ultrasound signal using a pressure transducer of a device (e.g., an IMD) and filtering the ultrasound signal using a filter coupled to the pressure transducer. This may improve measurement of a pressure signal by attenuating the ultrasound signal.

In some variations, a system may be configured to exchange wireless power or data only during a predetermined access period corresponding to a portion of a first device (e.g., IMD) spatial path not obstructed by tissue structures such as ribs or lungs. This may be useful for conserving energy of the external wireless device and/or the IMD, and minimizing tissue heating.

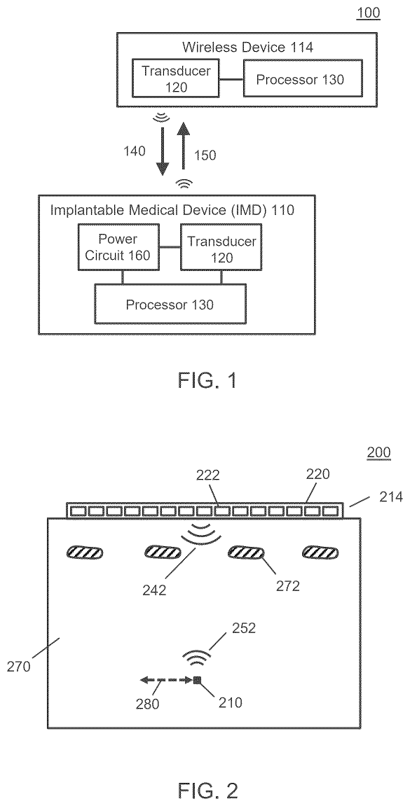

FIG. 1 is a schematic block diagram of an illustrative variation of a wireless system (100) comprising one or more devices (110, 114). The system (100) may comprise a first device (110) (e.g., wireless device, implantable medical device (IMD)), and a second device (114) (e.g., wireless device, external device). In some variations, the second device (114) may be disposed external to a body of a patient, or may be fully or partially implanted (e.g., under the skin). In some variations, one or more wireless signals such as a downlink signal (140) and an uplink signal (150) may be exchanged between the second device (114) and the first device (110). Downlink signals (140) may comprise one or more of power, data, and other signals transmitted by the second device (114) to the first device (110). Uplink signals (150) may comprise one or more of data and other signals received by the second device (114) from the first device (110).

In some variations, the downlink signal (140) and uplink signal (150) may be transmitted using one or more of mechanical waves (e.g., acoustic, ultrasonic, vibrational), magnetic fields (e.g., inductive), electric fields (e.g., capacitive), electromagnetic waves (e.g., RF, optical), galvanic coupling, surface waves, and the like. In some variations, the first device (110) and the second device (114) may comprise a transducer (120) configured to transmit and/or receive wireless signals.

In some variations, the first device (110) (e.g., IMD) may comprise a power circuit (160) comprising a power source (e.g., with energy storage capability) such as a battery, a capacitor, combinations thereof, and the like. In some variations, the power source of the first device may be recharged using wireless power transmitted by the second device (114). In some variations, one or more first devices (110) may be partially or fully powered or recharged through energy harvesting techniques including one or more of vibrational energy harvesting, motion of the heart, motion of blood vessel walls, blood flow, thermal energy harvesting, chemical energy harvesting, combinations thereof, and the like.

In some variations, the first device (110) and the second device (114) may comprise a processor (130) which may be configured to perform one or more of transmitting/receiving signals via the transducer (120), processing signals and/or data, combinations thereof, and the like. In some variations, the first device (110) may further comprise one or more of a sensor configured to sense a parameter such as a physiological parameter and a stimulator configured to stimulate tissue.

In some variations, the first device (110) may be implanted within, or on, one or more of a cardiac structure (e.g., heart chamber, heart valve), a vascular structure (e.g., pulmonary artery, any other blood vessel), and the like. In some variations, the first device (110) may be coupled (e.g., attached) to another implantable device (e.g., a prosthetic heart valve, a stent, and the like). In some variations, the first device (110) may move and/or rotate relative to the second device (114) due to one or more of the heart's pumping motion, breathing, coughing, sneezing, motion of the lungs, motion of other bodily organs or structures, motion of an implantable device, any movement of the first device, movement of the user (e.g., patient, nurse, physician) handling the second device, combinations thereof, and the like. In some variations, the wireless system (100) may comprise a plurality of first devices (110) and/or a plurality of second devices (114).

### B. Internal Device

Generally, a device disposed within a body of a patient (e.g., implantable medical device, wireless monitor, first device) described herein may be configured to perform one or more of sensing, monitoring, stimulation, therapy delivery, and the like, and may comprise one or more of the components described herein. In some variations, the device may comprise one or more of a transducer, power circuit, multiplexer, processor, memory, sensor, communication device (e.g., wireless device), and the like.

a. Transducer

Generally, the transducer (120) described here may be configured to convert a signal between a wireless energy modality and an electrical signal. In some variations, the transducer (120) may be configured to transmit and/or receive an uplink and/or downlink signal. A transducer (120), as described herein, may be a component of one or more of an first device (110) and a second device (114). In some variations, a transducer (120) may comprise a plurality of transducer elements. The transducer (120) may comprise one or more arrays (e.g., sub-arrays) that may be distinct or share common transducer elements.

In some variations, the transducer (120) may comprise one or more of an ultrasonic transducer, a radiofrequency (RF) transducer (e.g., a coil, an RF antenna), a capacitive transducer, combinations thereof, and the like. In some variations, an ultrasonic transducer may comprise one or more of a piezoelectric device, a capacitive micromachined ultrasonic transducer (CMUT), a piezoelectric micromachined ultrasonic transducer (PMUT), combinations thereof, and the like. In some variations, an ultrasonic transducer may convert one or more of pressure and force into an electrical signal, and vice versa. In some variations, the transducer (120) may comprise one or more ultrasonic transducers that may be of one or more types including, but not limited to, piston (e.g., rod, plate), cylindrical, ring, spherical (e.g., shell), flexural (e.g., bar, diaphragm), flextensional, combinations thereof, and the like. In some variations, a piezoelectric device may be made of one or more of lead zirconate titanate (PZT), PMN-PT, Barium titanate (BaTiO3), polyvinylidene difluoride (PVDF), Lithium niobate (LiNbO3), any derivates thereof, and the like. In some variations, an ultrasonic transducer may be configured to receive power at a frequency between about 20 kHz to about 20 MHz. The frequency ranges described herein may be enable the ultrasonic transducer to comprise millimeter or sub-millimeter dimensions. In some variations, the transducer (120) may comprise an RF transducer such as a coil or an antenna that may be configured to transmit and/or receive one or more of power, data, and other signals.

In some variations, the transducer (120) may comprise one or more transducer elements (e.g., one or more arrays or sub-arrays of transducer elements) configured to receive a downlink signal and/or transmit an uplink signal. For example, a transducer (120) of a second device (114) may comprise one or more arrays (e.g., sub-arrays) of ultrasonic transducer elements configured to transmit and/or receive ultrasonic signals.

In some variations, the transducer (120) comprising a plurality of transducer elements may be configured to perform a predetermined set of functions. For example, a first transducer element may be configured to recover wireless power, a second transducer element may be configured to receive data or signals, and a third transducer element may be configured to transmit data or signals. In some variations, a transducer may comprise a volume of less than about 10 cm³. Such transducer size may enable a compact (e.g., miniaturized) first device housing to aid minimally invasive delivery of the first device into the body via percutaneous or transcatheter techniques. In some variations, a transducer (e.g., an ultrasonic transducer) of an first device may be physically oriented (e.g., angled) and positioned towards a transducer of the second device. This may increase the consistency, reliability, and energy efficiency of wireless power and data exchange.

i. Ultrasonic Transducer Beam

In some variations, an ultrasonic transducer (120) of a wireless device (e.g., first device, second device) may comprise a plurality of ultrasonic transducer elements configured to achieve an aggregate radiation pattern (e.g., beam) with a wide acceptance angle (e.g., 3 dB beam width). In some variations, one or more ultrasonic transducer elements of the wireless device may have a set of different characteristics with respect to each other that in aggregate forms a radiation pattern with a wide acceptance angle. In some variations, a first device may be implanted in the body without precise knowledge of the orientation or position of the first device relative to a second device. For example, an orientation of one or more ultrasonic transducers of the first device or an orientation of a main lobe of one or more radiation patterns of the ultrasonic transducer may not be known to a second device. For example, a first device may rotate after implantation, either temporarily due to motion of the first device relative to a second device (e.g., due to a heartbeat, breathing, and the like) or the first device may slowly rotate over time relative to tissue (e.g., months or years), or both. The systems, devices, and methods described herein may overcome these challenges in aligning a second device to a first device to reliably transfer power and/or downlink signals.

In some variations, a set of characteristics may include, but is not limited to, a position, orientation, or angle of a transducer element relative to other elements (e.g., transducer element on a flat substrate or mounted on a specific structure at a predetermined angle with respect to each other), dimensions of a transducer element, material of a transducer element, poling direction of a piezoelectric element, poling direction relative to the electrode locations (e.g., side-electrode structures), combinations thereof, and the like.

In some variations, an ultrasonic transducer may comprise three ultrasonic transducer elements oriented at non-zero angles (e.g., orthogonal, at an angle of 30° with respect to each other, and the like) relative to each other. For example, by orienting three transducer elements orthogonal to each other, each transducer element may preferably receive wireless power from one of three orthogonal directions, thereby enabling one or more of power recovery from a plurality of directions (e.g., relatively omni-directional power recovery) and an aggregate radiation pattern with a wide acceptance angle. In some variations, a set of three ultrasonic transducer elements may be disposed on a substrate (e.g., a PCB) in a compact module (e.g., using 3D assembly).

b. Power Circuit

In some variations, one of more ultrasonic transducer elements of the first device may be interfaced to a power circuit for receiving power as described in further detail herein. Generally, a power circuit (160) as shown in FIG. 1 may be coupled to a transducer (120) of a first device (110), and may be configured to recover (e.g., condition) received wireless power, store energy, and supply power for different operations of the first device. In some variations, the power circuit (160) may comprise one or more energy storage elements (e.g., battery, capacitor) configured to store energy received by the transducer. The power circuit may be configured to control (e.g., regulate, limit) the power provided to one or more components of the first device.

In some variations, the power circuit (160) may be configured to convert alternating current (AC) voltage at the terminals of a transducer into a DC voltage (e.g., using a rectifier). In some variations, the power circuit (160) may be configured to recover wireless power received by a plurality of transducer elements located on a first device. For example, a power circuit coupled to a plurality of transducer elements may perform one or more of AC power combining, DC power combining, DC voltage combining, DC current combining, any combinations thereof, and the like.

In some variations, the power circuit (160) may comprise a power source comprising one or more of a capacitor, a super-capacitor, a rechargeable or secondary battery, a non-rechargeable or primary battery, combinations thereof, and the like. In some variations, the power circuit (160) may comprise a rechargeable battery for energy storage, along with a capacitor in parallel with the battery, where the capacitor may sink/supply at least a part of the current during charging/discharging transients of the rechargeable battery.

In some variations, the power circuit (160) may not include a device for storing energy, and the first device may be concurrently powered by another device (e.g., second device, another IMD, and the like) while the first device executes its functions. In some variations, power may be provided to a first device until it completes its functions and the first device may remain inactive until it is powered again.

In some variations, the systems, devices, and methods disclosed herein may comprise one or more systems, devices, and methods described in U.S. Pat. No. 9,544,068, filed on May 13, 2014, U.S. Pat. No. 10,177,606, filed on Sep. 30, 2016, U.S. Pat. No. 10,014,570, filed on Dec. 7, 2016, and U.S. Pat. No. 9,774,277 filed on Nov. 13, 2013, the contents of each of which are hereby incorporated by reference in its entirety.

c. Multiplexer Circuit

Generally, the multiplexer (e.g., multiplexer circuit) described here may be configured to decouple one or more of a power signal, data signal, and other signals in a first device. Decoupling signals may avoid interference between signals and ensure proper functioning of the first device. For example, a multiplexer in a first device may be configured to decouple a power signal from a data signal received from a second device such that the power signal is provided to the power circuit for power recovery and conditioning while the data signal is provided to the processor for data recovery.

In some variations, the multiplexer may comprise one or more of transmit/receive switches, passive devices (e.g., diodes, relays, MEMS circuits, blockers, passive switches), circulators, frequency selection (e.g., using filters, impedance matching networks), direct wired connections, combinations thereof, and the like.

d. Processor

Generally, the processor (e.g., CPU) described here may receive, transmit and/or process data and/or other signals, and/or control one or more components of the system (e.g., IMD). The processor may be configured to receive, process, compile, compute, store, access, read, write, and/or transmit data and/or other signals. Additionally, or alternatively, one or more elements of the processor of a first device (e.g., a sensing and processing circuit as discussed herein) may be configured to control one or more other elements of the processor (e.g., multiplexer circuit, demultiplexer circuit, and the like) and/or one or more components of a first device (e.g., transducer, power circuit, memory, sensor, and the like). A processor, as described herein, may be included in one or more of a first device, a second device, and the like.

In some variations, the processor may comprise a data communication circuit that may be a data receiver, which may be configured to access or receive data and/or other signals from one or more of a transducer, a sensor (e.g., pressure sensor) and a storage medium (e.g., memory, flash drive, memory card). For example, the processor may comprise one or more of a signal receiver (e.g., detecting an interrogation signal), an envelope detector circuit, an amplifier (e.g., a low-noise amplifier or LNA), a filter, a frequency detector circuit, a phase detector circuit, comparator circuits, decoder circuits, combinations thereof, and the like, configured to receive data and/or signals through the transducer. In some variations, the processor of a first device may comprise monitoring circuits configured to monitor one or more of the voltage, current, power and energy of the first device.

In some variations, the processor may comprise any suitable processing device configured to run and/or execute a set of instructions or code and may include one or more data processors, image processors, graphics processing units (GPU), physics processing units, digital signal processors (DSP), analog signal processors, mixed-signal processors, machine learning processors, deep learning processors, finite state machines (FSM), compression processors (e.g., data compression to reduce data rate and/or memory requirements), encryption processors (e.g., for secure wireless data and/or power transfer), and/or central processing units (CPU). The processor may comprise, for example, a general purpose processor, Field Programmable Gate Array (FPGA), an Application Specific Integrated Circuit (ASIC), a processor board, and/or the like. The processor may be configured to run and/or execute application processes and/or other modules, processes and/or functions associated with the system. The underlying device technologies may be provided in a variety of component types (e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, and/or the like.

The systems, devices, and/or methods described herein may be performed by software (executed on hardware), hardware, or a combination thereof. Hardware modules may include, for example, a general-purpose processor (or microprocessor or microcontroller), a field programmable gate array (FPGA), and/or an application specific integrated circuit (ASIC). Software modules (executed on hardware) may be expressed in a variety of software languages (e.g., computer code), including C, C++, Java®, Python, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

In some variations, the processor may be configured to process a signal (e.g., an interrogation signal) and take an action (e.g., generate a feedback signal). For example, the processor may comprise a sensing and processing circuit as described in detail herein. In some variations, the processor of a first device may be configured to process an interrogation signal which may encode an identification (ID) number of the first device and decode the ID number. In such variations, the processor may be configured to decode or extract an ID number from the received interrogation signal and perform an action (e.g., generate a feedback signal, take no action, and the like) depending on whether the ID in the interrogation signal matches the ID of the first device. In some variations, a processor of a first device may comprise one or more of an envelope detection circuit, an energy detector circuit, a power detector circuit, a voltage sensor, a time-to-digital converter (TDC) circuit, an integrator circuit, a sampling circuit, an analog-to-digital converter (ADC) circuit, a timer circuit, a clock, a counter, an oscillator, a phase-locked loop (PLL), a frequency locked loop (FLL), combinations thereof, and the like, for processing an interrogation signal received from a second device to generate a feedback signal.

In some variations, the processor of a wireless device may be configured to process a signal (e.g., a feedback signal), generate data (e.g., feedback signal data) and determine a transducer configuration of the wireless device (e.g., a sub-array) for powering a first device, as described in detail herein. For example, the processor may comprise an amplifier, a phase detector, a frequency detector, a digital signal processor, an integrator, an adder circuit, a multiplier circuit, a finite state machine, combinations thereof, and the like, for performing such computations.

In some variations, the processor may comprise a data communication circuit that may be a data transmitter, which may be configured to generate or transmit data and/or other signals through one or more of a transducer, a storage medium, and the like. For example, a processor of a first device may comprise one or more of a signal transmitter, an uplink data transmitter, an oscillator, a power amplifier, a mixer, an impedance matching circuit, a switch, a driver circuit, combinations thereof, and the like, to generate or transmit data and/or signals via the transducer.

In some variations, the processor may be configured to control one or more elements in a first device and/or a second device. For example, the processor may be configured to control a first device to generate a feedback signal or perform a sensing function depending on a command received from the second device via a downlink signal.

In some variations, a first processor may be a component of a first device and a second processor may be a component of a second device. In such variations, a first device may be configured to receive an interrogation signal, and the first processor may be configured to process the interrogation signal and generate a feedback signal. The second processor may be configured to process the feedback signal received by the second device, generate feedback signal data, and determine a transducer configuration of the second device as described in detail herein. In some variations, the first processor of the first device may be configured to process one or more interrogation signals and determine a transducer configuration based on the interrogation signals (e.g., a transducer configuration of the second device that resulted in the maximum power of the interrogation signal at the first device).

e. Memory

Generally, the first device and/or the second device described here may comprise a memory configured to store data and/or information. In some variations, the memory comprise one or more types including, but not limited to, random access memory (RAM), static RAM (SRAM), dynamic RAM (DRAM), resistive random-access memory (ReRAM or RRAM), magnetoresistive random-access memory (MRAM), ferroelectric random-access memory (FRAM), standard-cell based memory (SCM), shift registers, read-only memory (ROM), programmable read-only memory (PROM), erasable programmable read-only memory (EPROM), electrically erasable programmable read-only memory (EEPROM), flash memory (e.g., NOR, NAND), embedded flash, volatile memory, non-volatile memory, one time programmable (OTP) memory, combinations thereof, and the like.

In some variations, the memory may be configured to store instructions and/or data to cause the processor to execute modules, processes, and/or functions (e.g., executing a search algorithm) associated with a first device and/or a second device. Some variations described herein may relate to a computer storage product with a non-transitory computer-readable medium (also may be referred to as a non-transitory processor-readable medium) having instructions or computer code thereon for performing various computer-implemented operations. The computer-readable medium (or processor-readable medium) may be non-transitory in the sense that it may not include transitory propagating signals per se (e.g., a propagating electromagnetic wave carrying data on a transmission medium such as space or a cable). The media and computer code (also may be referred to as code or algorithm) may be those designed and constructed for the specific purpose or purposes.

In some variations, the memory may be configured to store sensor data, received data and/or data generated by the first device and/or the second device. In some variations, the memory of a first device may be configured to store data generated upon processing signals sensed by a sensor (e.g., blood pressure data sensed by a pressure sensor that may be included in a first device). In some variations, the memory of the first device may be configured to store one or more of a parameter of the interrogation signal, a parameter of the feedback signal, a parameter related to the power received by the first device, a parameter related to the movement and/or rotation or trajectory of a moving IMD, and the like.

In some variations, the memory of the second device may be configured to store one or more of a parameter of the interrogation signal, a parameter of the feedback signal, feedback signal data, data corresponding to a transducer configuration, patient data, wireless system data (e.g., number and/or location of IMDs, spatial path of one or more IMDs, one or more identification (ID) numbers corresponding to one or more IMDs), combinations and derivatives thereof, and the like. In some variations, the memory of the first device and/or the second device may be configured to store image data including 2D, 3D, Doppler, and any other data generated from imaging (e.g., an image of the rib cage or tissue, echocardiography images, MRI images) of the patient. In some variations, the memory may be configured to store data temporarily or permanently.

f. Sensor

Generally, the sensors described here may be configured to sense or measure one or more parameters such as, but not limited to, physiological parameters of a patient. In some variations, the sensor may comprise one or more of a pressure sensor, flow sensor, transducer (e.g., an ultrasonic transducer, an infrared/optical photodiode, an infrared/optical LED, an RF antenna, an RF coil), temperature sensor, electrical sensor (e.g., using electrodes for measuring impedance, electromyogram or EMG, electrocardiogram or ECG, and the like), magnetic sensor (e.g., RF coil), electromagnetic sensor (e.g., infrared photodiode, optical photodiode, RF antenna), neural sensor (e.g., for sensing neural action potentials), force sensor (e.g., a strain gauge), flow or velocity sensor (e.g., hot wire anemometer, vortex flowmeter), acceleration sensor (e.g., accelerometer), an activity sensor (e.g., to monitor patient activity levels), chemical sensor (e.g., pH sensors, protein sensor, glucose sensor), oxygen sensor (e.g., pulse oximetry sensor, myocardial oxygen consumption sensor), audio sensor (e.g., a microphone to detect heart murmurs, prosthetic valve murmurs, auscultation), sensor for sensing other physiological parameters (e.g., sensors to sense heart rate, breathing rate, arrhythmia, motion of heart walls), a stimulator (e.g., for stimulation and/or pacing function), combinations thereof, and the like.

In some variations, one or more pressure sensors (also referred to as a pressure transducer) may be used for monitoring cardiovascular diseases such as heart failure. In some variations, one or more pressure sensors may include, but not be limited to, an absolute pressure sensor, gauge pressure sensor, sealed pressure sensor, differential pressure sensor, atmospheric pressure sensor, combinations thereof, and the like. In some variations, one or more pressure sensors may be based upon one or more pressure-sensing technologies including, but not limited to, resistive (e.g., piezoresistive, using a strain gauge or a membrane to create a pressure-sensitive resistance), capacitive (e.g., using a diaphragm or a membrane to create a pressure-sensitive capacitance), piezoelectric, optical, resonant (e.g., pressure-sensitive resonance frequency of a structure), combinations thereof, and the like. In some variations, a pressure sensor may be manufactured using Micro-Electro-Mechanical Systems (MEMS) technology.

In some variations, the sensor may comprise a stimulator (e.g., an electrical stimulator) used for stimulating muscles and/or neurons or nerves of the body. For example, one or more stimulators may be configured to stimulate a ventricular wall for pacing and/or cardiac resynchronization.

g. Spatial Path

A spatial path of a first device (e.g., IMD) may generally refer to a set of positions (e.g., a path, trajectory) and/or a set of orientations traversed by a first device relative to a second device (e.g., an external wireless device). For example, the first device may move and/or rotate relative to the second device. A spatial path of a first device may comprise one or more of a line, a curved path, a rotation, a tilt, combinations thereof, and the like. In some variations where a first device may be implanted in or near cardiac tissue or a cardiovascular structure, the spatial path of the first device may also be referred to as a cardiac path. Movement of the first device may be due to one or more of the heart's pumping motion, breathing, motion of the lungs, motion of other bodily organs or structures, movement of the external wireless device, movement of the user (e.g., patient, nurse, physician) handling the external wireless device, combinations thereof, and the like.

h. Operation Modes

A first device (e.g., IMD), as described herein, may be configured to operate in one or more modes including, but not limited to, a stimulation mode, a sensing mode, a wireless downlink mode, a wireless uplink mode, a sleep mode, combinations thereof, and the like. The sensing mode may comprise one or more of sensing or sampling (e.g., periodically) a parameter (e.g., pressure) to generate a sensor signal, conditioning the sensor signal, digitizing the sensor signal to generate a digitized signal, combinations thereof, and the like. The wireless downlink mode may comprise a mode where the first device may be configured to receive one or more of power, data, one or more signals (e.g., an interrogation signal), one or more commands, combinations thereof, and the like. The wireless uplink mode may comprise a mode where the first device may be configured to transmit or generate one or more of uplink data (e.g., processed data), one or more wireless signals (e.g., an active uplink signal, a reflection signal, a modulated backscatter signal, and the like), combinations thereof, and the like. In a sleep mode, the first device may be configured to not perform any active functions (e.g., sensing, stimulation, etc.), and wait for instructions or interrogation signals from a second device.

i. First Device Placement in the Body

Generally, the implantable devices described here may be configured to be disposed (e.g., implanted) inside a patient or an animal. In some variations, a first device, as described herein, may be a standalone device. In some variations, a first device, as described herein, may be coupled (e.g., attached) to another device disposed in the body. For example, one or more first devices may be coupled to a prosthetic heart valve or a stent. As another example, one or more first devices may be coupled to one or more of a pulse generator and one or more leads of a pacemaker, an implantable cardioverter defibrillator, and/or cardiac resynchronization therapy devices.

In some variations, a first device may be coupled to one or more of prosthetic heart valves, prosthetic heart valve conduit, valve leaflet coaptation devices, annuloplasty rings, valve repair devices (e.g., clips, pledgets), septal occluders, appendage occluders, ventricular assist devices, pacemakers (e.g., including leads, pulse generator), implantable cardioverter defibrillators (e.g., including leads, pulse generator), cardiac resynchronization therapy devices (e.g., including leads, pulse generator), insertable cardiac monitors, stents (e.g., coronary or peripheral stents, fabric stents, metal stents), stent grafts, scaffolds, embolic protection devices, embolization coils, endovascular plugs, vascular patches, vascular closure devices, interatrial shunts, parachute devices for treating heart failure, cardiac loop recorders, combinations thereof, and the like. For example, a prosthetic heart valve may comprise one or more of a transcatheter heart valve (THV), self-expandable THV, balloon expandable THV, surgical bioprosthetic heart valve, mechanical valve, and the like.

Generally, the implantable devices described here may be located in or near any region in the body, including but not limited to heart valves (e.g., aortic valve, mitral valve), heart chambers (e.g., left ventricle, left atrium, right ventricle, right atrium), blood vessels (e.g., pulmonary artery, aorta, superficial femoral artery, coronary artery, pulmonary vein, and the like), heart tissue (e.g., heart muscle or wall, septum), gastrointestinal tract (e.g., stomach, esophagus), bladder, combinations thereof, and the like.

### C. Second Device

Generally, as used herein, a second device (e.g., wireless device, external wireless device) may refer to any device that is physically separate from one or more first devices (e.g., IMDs). In some variations, the second device (114) may comprise a transducer (120) and a processor (130), as illustrated for example in FIG. 1. In some variations, a second device may comprise memory as described herein. In some variations, a second device may comprise a battery for storing energy that may be used for wirelessly powering and/or communicating with one or more first devices and/or for communicating with one or more other second devices (e.g., tablet, phone, laptop, computer, server, database, network).

In some variations, the transducer (120) of the second device (114) may comprise a plurality of ultrasonic transducer elements which may comprise a plurality of configurations to exchange wireless signals (transmit and/or receive) with one or more IMDs, as described in detail herein. In some variations, the second device may comprise one or more transducer arrays (e.g., sub-arrays, transducer elements). In some variations, one or more transducers or transducer elements of a second device may comprise one or more of an ultrasonic transducer, a radiofrequency (RF) transducer (e.g., a coil, an RF antenna), a capacitive transducer, combinations thereof, and the like. In some variations, the processor (130) of the second device (114) may be configured to process a wireless signal (e.g., a feedback signal) received from the first device.

In some variations, a second device may be configured to perform one or more functions including, but not limited to, transmitting one or more of wireless power, data, and other signals (e.g., interrogation signal) to one or more first devices (e.g., IMDs), receiving one or more of wireless data and other signals (e.g., feedback signal) from one or more first devices, processing data and/or signals (e.g., processing feedback signal), performing sensing and/or actuation (e.g., measuring blood pressure, heart rate, heart rate variability, ECG, EKG, thoracic impedance, breathing rate or respiration, patient activity levels, heart sounds, lung sounds, temperature, body weight, blood glucose, blood oxygen), storing data or information in memory, communicating with other wireless devices (e.g., tablet, phone, computer) via wires and/or using wireless links (e.g., Bluetooth), displaying or providing data or information (e.g., visual display on a screen or a monitor, audio signals), generating alerts/notifications (e.g., visual, audio, vibration) to a user (e.g., patient, doctor), combinations thereof, and the like.

In some variations, the second device may be disposed in one or more locations including, but not limited to, outside the body (e.g., as a wearable device, a strap, a belt, a handheld device, a probe coupled to a measurement setup, a device placed on skin, a device attached to skin using an adhesive, a device attached to skin using other techniques, a device not touching the patient, a laptop, a computer, a mobile phone, a smartwatch, and the like), permanently implanted inside the body (e.g., implanted under the skin, along the outer wall of an organ), temporarily implanted inside the body (e.g., located on a catheter or a probe inserted through a blood vessel, esophagus or the chest wall, used during surgery or procedure), combinations thereof, and the like. In some variations, the second device may have different shapes or forms including, but not limited to, planar, conformal to the body or an organ, flexible, stretchable, flat, shaped like a probe, and the like. In some variations, a second device may perform a function for another second device (e.g., processing feedback signals). For example, a second device placed on the body of a patient (e.g., placed on the chest) may communicate feedback signal data generated from feedback signals received from one or more IMDs to another second device such as a laptop, a tablet, a cell phone, and the like. This another second device may process the feedback signal data and execute a search algorithm and/or perform computations (e.g., determine a transducer configuration for powering the first device).

In some variations, the second device may further comprise a communication device configured to permit a user and/or health care professional to control one or more of the devices of the wireless system. The communication device may comprise a network interface configured to connect the second device to another system (e.g., Internet, remote server, database) by wired or wireless connection. In some variations, the second device may be in communication with other devices (e.g., cell phone, tablet, computer, smartwatch, and the like) via one or more wired and/or wireless networks. In some variations, the network interface may comprise one or more of a radiofrequency (RF) receiver, RF transmitter, an optical (e.g., infrared) receiver, optical transmitter, an acoustic or ultrasonic receiver and transmitter, and the like, configured to communicate with one or more devices and/or networks. The network interface may communicate by wires and/or wirelessly with one or more of the wireless device, network, database, and server.

The network interface may comprise RF circuitry configured to receive and/or transmit RF signals. The RF circuitry may convert electrical signals to electromagnetic signals (and vice versa) and communicate with communication networks and other communication devices via the electromagnetic signals. The RF circuitry may comprise well-known circuitry for performing these functions including, but not limited to, an antenna system, an RF transceiver, one or more amplifiers, a tuner, one or more oscillators, a mixer, a digital signal processor, a CODEC chipset, a subscriber identity module (SIM) card, memory, and so forth.

Wireless communication through any of the devices described herein may use any of plurality of communication modalities, standards, protocols and technologies including, but not limited to, Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (WiFi) (e.g., IEEE 802.11a, IEEE 802.11b, IEEE 802.11g, IEEE 802.11n, and the like), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol. In some variations, the devices herein may directly communicate with each other without transmitting data through a network (e.g., through NFC, Bluetooth, WiFi, RFID, and the like).

The communication device may further comprise a user interface configured to permit a user (e.g., patient, subject, partner, family member, health care professional, etc.) to control the second device. The communication device may permit a user to interact with and/or control a second device directly and/or remotely. For example, a user interface of the second device may include an input device for a user to input commands and an for a user to receive output (e.g., blood pressure readings on a display device).

In some variations, a second device may comprise an output device and a user interface. An output device of the user interface may output one or more of data corresponding to the coupling of a second device to tissue or skin, data corresponding to the wireless link between the second device and the first device (e.g., reliable link been established), movement, rotation and/or trajectory of one or more IMDs, and the like, and may comprise one or more of a display device and audio device. Data analysis generated by a server may be displayed by the output device (e.g., display) of the second device. Data used in selecting a transducer configuration or ensuring that a second device is optimally coupled to tissue may be received through a communication device and output visually and/or audibly through one or more output devices of the second device. In some variations, an output device may comprise a display device including at least one of a light emitting diode (LED), liquid crystal display (LCD), electroluminescent display (ELD), plasma display panel (PDP), thin film transistor (TFT), organic light emitting diodes (OLED), electronic paper/e-ink display, laser display, and/or holographic display.

An audio device may audibly output one or more of any data, commands, instructions, prompts, alarms, notifications, and the like. For example, the audio device may output an audible alarm when the link between a first device and a second device is disturbed or broken and manual adjustment by a user is prompted. In some variations, an audio device may comprise at least one of a speaker, piezoelectric audio device, magnetostrictive speaker, and/or digital speaker. In some variations, a user may communicate with other users using the audio device and a communication channel. For example, a user may form an audio communication channel (e.g., VoIP call) with a remote health care professional using the second device and another device.

In some variations, the user interface may comprise an input device (e.g., touch screen) and output device (e.g., display device) and be configured to receive input data from one or more of the first device, a second device, network, database, and server. For example, user control of an input device (e.g., keyboard, buttons, touch screen) may be received by the user interface and may then be processed by processor and memory for the user interface to output a control signal to the first device. Some variations of an input device may comprise at least one switch configured to generate a control signal. For example, an input device may comprise a touch surface for a user to provide input (e.g., finger contact to the touch surface) corresponding to a control signal. An input device comprising a touch surface may be configured to detect contact and movement on the touch surface using any of a plurality of touch sensitivity technologies including capacitive, resistive, infrared, optical imaging, dispersive signal, acoustic pulse recognition, and surface acoustic wave technologies. In variations of an input device comprising at least one switch, a switch may comprise, for example, at least one of a button (e.g., hard key, soft key), touch surface, keyboard, analog stick (e.g., joystick), directional pad, mouse, trackball, jog dial, step switch, rocker switch, pointer device (e.g., stylus), motion sensor, image sensor, and microphone. A motion sensor may receive user movement data from an optical sensor and classify a user gesture as a control signal. A microphone may receive audio data and recognize a user voice as a control signal.

A haptic device may be incorporated into one or more of the input and output devices to provide additional sensory output (e.g., force feedback) to the user. For example, a haptic device may generate a tactile response (e.g., vibration) to confirm user input to an input device (e.g., touch surface). As another example, haptic feedback may notify that user input is overridden by the second device.

a. Sub-array

A sub-array may generally refer to any subset of a plurality of transducer elements of the second device. In some variations, a sub-array may comprise one or more of a set of adjacent transducer elements, a set of alternating transducer elements (e.g., every second element), a set of every ‘n^{th}’ transducer elements, or any subset of transducer elements of a transducer array. For example, a sub-array may comprise a set of transducer elements selected for efficiently transferring wireless power to a first device based on a feedback signal, as described in detail herein. In some variations, a sub-array may comprise a single transducer element of the second device. In some variations, a sub-array may comprise all transducer elements of the second device.

In some variations, sub-arrays may comprise a disjoint set of transducer elements. For example, a second device may comprise a linear 1D array with array elements labeled 1, 2, 3, and so on, where sub-arrays may be comprised of element numbers 1-8, 9-16, 17-24, and so on. In some variations, sub-arrays may comprise an overlapping set of transducer elements. For example, for the example of a linear 1D array, sub-arrays may be comprised of element numbers 1-8, 2-9, 3-10, and so on. In some variations, sub-arrays may have different sizes. For example, different sub-arrays of the same second device may comprise one or more of different number of transducer elements (e.g., some sub-arrays may comprise 4 transducer elements, some sub-arrays may comprise 16 transducer elements), transducer elements with different sizes, combinations thereof, and the like. In some variations, the selection of transducer elements for a predetermined sub-array of the second device may be based upon feedback signal data, as described in detail herein.

b. Transducer Configuration

A transducer configuration may generally refer to one or more transducer elements of a second device configured to exchange one or more of wireless power and data with a first device (e.g., for recharging a power source of a first device). A transducer configuration may also refer to the parameters and settings of the one or more transducer elements configured to drive signal transmission (e.g., the frequency, amplitude, phase, time delay, duration, and the like, with which the one or more transducer elements may be configured to transmit signals) and signal reception (e.g., phase shift, time delay, gain, and the like, with which the one or more transducer elements may be configured to receive signals). In some variations, a transducer configuration may be selected by a processor of a second device based on a feedback signal received from a first device.

In some variations, a transducer configuration configured to transmit wireless signals to a first device may be referred to as a transmit transducer configuration (TTC). In some variations, a transducer configuration configured to receive wireless signals from a first device may be referred to as a receive transducer configuration (RTC). In some variations, a transducer configuration selected by a processor of a second device based on a feedback signal received from a first device may be referred to as an optimal transducer configuration (OTC) that may be improved relative to a default transducer configuration, but which is not necessarily the most optimal transducer configuration. In some variations, a set of transducer elements of the wireless device, along with the driving signals for each of those transducer elements, that may be selectively configured for powering a wireless monitor and/or transmitting other downlink signals to the wireless monitor, may be collectively referred to as a sub-array powering snapshot. In some variations, a set of transducer elements of the wireless device configured to receive uplink signals (e.g., data) from an IMD, along with parameters related to receiving signals, or conditioning received signals, such as gain, phase-shift, delay, filtering, time window for receiving signals, and the like, may be collectively referred to as a sub-array uplink data snapshot.

c. User Prompt

A user prompt (also referred to as user feedback) may generally refer to one or more instructions, notifications, recommendations, alerts, and the like provided by a second device to a user. A user prompt may serve a number of purposes including, but not limited to communicating data about the state of charge (SoC) and/or depth of discharge (DoD) of the first device's and/or the second device's battery, asking a user to recharge the second device battery, communicating data about the data transfer between a first device and a second device (e.g., percent data transfer complete), asking a user to manually adjust or reposition a second device on a patient's body, combinations thereof, and the like. In some variations, a user prompt may be provided using one or more of visual instructions, audio instructions, vibrations, notifications (e.g., alert, push notification, email, and the like, on the phone, computer, and the like), combinations thereof, and the like. Variations of the communication device, user interface, input device, output device, etc., as described herein, may be used for providing a user prompt.

In some variations, the user prompt (e.g., visual instructions) may comprise one or more of an image, photo, and stylized representation (e.g., schematic, cartoon, diagram) of a patient's chest (e.g., showing one or more of chest, arms, neck, head), current device configuration (e.g., position, angle, tilt, and the like) of the second device, target device configuration (e.g., position, angle, rotation, tilt, and the like) of the second device, a map showing current/target positions, instructions displayed in the form of text (e.g., a sentence asking the user to move the second device towards the patient's left arm, right arm, head, and the like; numbers or percentage representing power received by a first device, SoC and/or DoD of the battery, and the like), arrows directing a user to move, rotate and/or adjust a second device, LEDs (e.g., steady, blinking), combinations thereof, and the like. For example, in some variations, the current position, as well as a target position, of the wireless device may be overlaid on the image of the chest. A user may be instructed to move the second device until it reaches the target position.

In some variations, audio instructions may comprise one or more of voice commands (e.g., asking the user to move the second device towards the patient's left arm, asking the user to recharge the second device battery, notifying a user of completed data transfer from a first device to a second device), beeps, alarms, combinations thereof, and the like.

d. Network

In some variations, the systems, devices, and methods described herein may be in communication with other wireless devices via, for example, one or more networks, each of which may be any type of network (e.g., wired network, wireless network). The communication may or may not be encrypted. A wireless network may refer to any type of digital network that is not connected by cables of any kind. Examples of wireless communication in a wireless network include, but are not limited to cellular, radio, satellite, and microwave communication. However, a wireless network may be connected to a wired network in order to interface with the Internet, other carrier voice and data networks, business networks, and personal networks. A wired network is typically carried over copper twisted pair, coaxial cable and/or fiber optic cables. There are many different types of wired networks including wide area networks (WAN), metropolitan area networks (MAN), local area networks (LAN), Internet area networks (IAN), campus area networks (CAN), global area networks (GAN), like the Internet, and virtual private networks (VPN). Hereinafter, network refers to any combination of wireless, wired, public and private data networks that are typically interconnected through the Internet, to provide a unified networking and information access system.

Cellular communication may encompass technologies such as GSM, PCS, CDMA or GPRS, W-CDMA, EDGE or CDMA2000, LTE, WiMAX, and 5G networking standards. Some wireless network deployments combine networks from multiple cellular networks or use a mix of cellular, Wi-Fi, and satellite communication. In some variations, the network may be used for remote processing of any data or information used by the wireless system described herein. For example, a processor that may process any data or information related to the wireless system may be located in the same housing as the first device and/or in the same housing as a second device, in a separate housing in the same room or building as the first device, in a remote location from the first device and the second device (e.g., a different building, city, country), any combinations thereof, and the like. Processing of data or information related to the wireless system may be performed in real-time as the data (e.g., feedback signal data, physiological data) is received or recorded, or it may be performed at a different time.

### D. Wireless Signals

Wireless signals as used herein may generally refer to any wireless signal exchanged between at least two devices such as a first device and a second device. In some variations, a wireless signal may comprise one or more of a power signal, a downlink data signal, an interrogation signal, a feedback signal, an uplink data signal, a reflection signal, a backscatter signal, and the like. For example, in some variations, a wireless signal generated by a first device may include a reflection signal or a backscatter signal from the first device, generated upon incidence of a downlink signal such as an interrogation signal onto the first device.

a. Interrogation Signal

An interrogation signal may generally refer to any signal transmitted by a second device during an interrogation process of a first device, or one or more other methods, as described in more detail herein. For example, an interrogation signal may refer to any signal transmitted by a sub-array of a second device configured to elicit a feedback signal from a first device. In some variations, an interrogation signal may be one or more of a power signal configured to transfer wireless power to a first device, a downlink data signal configured to transfer data/commands to a first device, and any other signal configured to elicit a feedback signal from a first device, combinations thereof, and the like.

In some variations, an interrogation signal may be generated using one or more of mechanical waves (e.g., ultrasonic, acoustic, vibrational), magnetic fields (e.g., inductive), electric fields (e.g., capacitive), electromagnetic waves (e.g., RF, optical), galvanic coupling, surface waves, and the like. In some variations, an interrogation signal may be generated in the form of a continuous wave (CW) signal or a pulsed wave (PW) signal. In some variations, the interrogation signal may be generated using any known digital or analog modulation techniques such as ASK, FSK, PSK, AM, FM, PM, pulse modulation, PAM, PIMD, PPM, PCM, PDM, and the like. In some variations, an ultrasonic interrogation signal may comprise a carrier frequency of between about 20 kHz to about 20 MHz.

In some variations, an interrogation signal may encode a unique identification (ID) number or code corresponding to one or more wireless devices (e.g., IMDs). For example, an ID number may be configured to command one or more predetermined IMDs to respond to an interrogation signal. In some variations, an interrogation signal may encode a command corresponding to one or more functions of a first device. For example, in some variations, an interrogation signal may encode a command, upon receiving which, a first device may configure itself to transmit a feedback signal to a second device. In some variations, an interrogation signal may encode a command, upon receiving which, a first device may configure itself to receive wireless power from a second device and/or recharge its power source such as a battery or a capacitor. In some variations, an interrogation signal may encode a command, upon receiving which, a first device may configure itself to transmit data to a second device via an uplink signal. In some variations, an interrogation signal may encode a command, upon receiving which, a first device may configure itself to operate in one or more modes of operation such as sensing mode, a stimulation mode, sleep mode, combinations thereof, and the like.

b. Feedback Signal

A feedback signal may generally refer to any signal received by a second device from a first device. In some variations, a feedback signal may be generated in response to another signal (e.g., interrogation signal). In some variations, a first device (e.g., an IMD) may be configured to transmit one or more feedback signals without being interrogated by a second device. For instance, a first device may be configured to periodically transmit feedback signals, which may be also be referred to as beacon signals in some variations.

In some variations, the feedback signal may comprise parameters (e.g., type, waveform shape, modulation, and the like) similar to those described with respect to the interrogation signal. For example, a feedback signal may comprise an ultrasonic pulse with a carrier frequency of between about 20 kHz to about 20 MHz.

In some variations, a feedback signal transmitted by a first device, in response to receiving an interrogation signal, may comprise one or more pulses. For example, in some variations, after receiving an interrogation signal, a first device may transmit a single ultrasonic pulse (e.g., comprising one or more cycles of a carrier frequency), or the first device may periodically transmit a plurality of ultrasonic pulses. Such an ultrasonic pulse may be used by a second device for triangulation or localization of the first device and/or estimating a link gain between the second device and the first device, as described in more detail herein.

In some variations, a feedback signal may comprise data encoded using any modulation technique (e.g., digital modulation). For example, in some variations, a first device may encode onto a feedback signal, one or more of the following including, but not limited to, the power or voltage received by one or more transducers of the first device due to an interrogation signal (e.g., after digitization of the power or voltage), the first device battery and/or capacitor voltage, energy state of the first device, stored energy on a power source of the first device (e.g., battery, capacitor), battery charging current, DC voltage generated by the first device's power circuit after rectifying an interrogation signal, combinations thereof, and the like. As another example, in some variations, a first device may encode a unique ID or code onto a feedback signal. In some variations, a feedback signal may encode a time delay. For example, in some variations, a feedback signal may encode the time delay (e.g., after digitization) between receipt of an interrogation signal from a second device and transmission of the feedback signal to a second device.

In some variations, a feedback signal may comprise one or more of a reflection signal and a backscatter signal. These signals may be generated upon reflection or backscattering of an interrogation signal, or any other signal transmitted by the second device, off one or more first devices and/or one or more tissue structures (such as ribs, lungs, boundaries between two tissue types, and the like). Reflections from a first device may comprise one or more reflections from one or more of the housing, coating or encapsulation of the first device, the first device transducer (e.g., ultrasonic transducer), surface of a first device (e.g., front, back, side, outer, inner), any part of a first device, combinations thereof, and the like. In some variations, the reflection signals may comprise ultrasonic reflection signals generated upon reflection of an ultrasonic signal transmitted by a sub-array of the second device into tissue.

c. Feedback Signal Data

Feedback signal data may generally refer to any property of the feedback signal as received by the second device, and/or any data generated upon processing of the feedback signal by a processor of the second device. In some variations, such a property of the feedback signal may comprise one or more of a phase, an arrival time, a time delay, an amplitude, an intensity, a power or energy, a frequency, number of pulses, any data or information that may be encoded onto the feedback signal (e.g., digitized battery voltage of the first device, a unique ID of a first device, and the like), combinations or derivatives thereof, and the like. Feedback signal data may be generated corresponding to one or more transducer elements of the second device. In some variations, feedback signal data may comprise data obtained from processing the feedback signal received at the current time and/or in the past.

### E. Wireless Power and Data Exchange

Described herein are systems configured to exchange wireless power or data between two devices, such as between an implantable medical device (IMD) and a second device (e.g., an external wireless device). Localization of the first device (i.e., estimating a location of the first device in tissue) may help the two devices reliably and efficiently exchange power or data since a precise location of the first device may not be known after implantation. Once the location of the first device is determined, wireless signals may be focused relative to the location of the first device. This may be especially important for exchanging ultrasonic power or data due to the low wavelength of ultrasound in tissue, but may also be useful for other wireless system such as those using RF power or data.

FIG. 2 is an illustrative variation of a system comprising a first device (210) implanted in the heart, surrounded by tissue (270) and ribs (272), along with an external second device (214) comprising one or more arrays (220) of transducer elements (222). In some variations, the second device (214) may be placed on a patient's chest. The second device (214) may be configured to transmit a downlink signal such as an interrogation signal (242) to the first device (210). The first device (210) may be configured to generate a wireless signal such as a feedback signal (252) comprising one or more of an uplink signal transmitted by the first device (210), a reflection signal from the first device (210), a backscatter signal from the first device (210), and the like. In some variations, the first device (210) may move relative to the second device (214) along a spatial path (280) or a periodic trajectory.

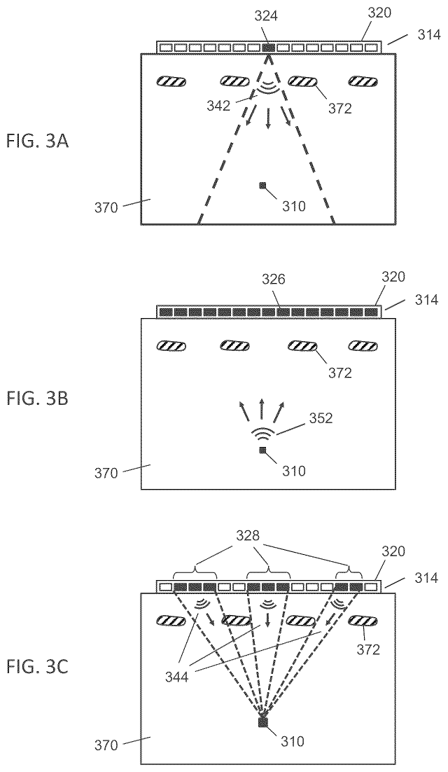

FIGS. 3A, 3B, and 3C depict an illustrative variation of a system configured to exchange wireless power or data between a first device (310) and a second device (314). The system may comprise a first device, (310), and a second device (314), where the second device (314) may comprise a processor (not shown) and a transducer array (320), comprising a plurality of sub-arrays. In some variations the transducer array (320) may comprise an ultrasound transducer array. In some variations, the first device (310) may be enclosed (e.g., surrounded) by tissue (370) and ribs (372). In some variations, a first sub-array (324) of the transducer array (320) may be configured to transmit an interrogation signal (342) to the first device (310), as shown in FIG. 3A. For example, the first sub-array (324) may comprise a single transducer element, as highlighted in FIG. 3A, or a subset of transducer elements of the transducer array (320). In some variations, a second sub-array (326) may be configured to receive a feedback signal (352) from the first device (310), as shown in FIG. 3B. For example, the second sub-array (326) may comprise each of the transducer elements of the transducer array (320), as highlighted in FIG. 3B, or a subset of transducer elements of the transducer array (320). In some variations, a processor of the second device may be configured to select a transducer configuration (328), as shown in FIG. 3C, based on the feedback signal (352) received by the second sub-array (326). In some variations, as shown in FIG. 3C, the transducer configuration (328) may be configured to transmit a power signal (344) to the first device (310). In general, the transducer configuration (328) may be configured to exchange one or more of wireless power and data with the first device (310). Such a system and process may be useful for optimizing the link efficiency and reliability of power and data transfer between a first device and a second device.

a. Interrogation Signal

Different variations of an interrogation signal used for interrogation of a first device are described herein. In some variations, performing interrogation of a first device within a limited time period may be advantageous in order to complete a power/data transfer process quickly. In some of these variations, the wireless system may be configured such that the first device is able to quickly and reliably detect when a second device sends an interrogation signal to it. However, since the exact location of a first device may initially be unknown, and due to the presence of heterogeneous tissue structures and tissue losses, the intensity of an interrogation signal at the first device may be below the first device's detection threshold. For example, if a first device is located in or near the heart of a patient, and an ultrasonic signal is configured for interrogation of the first device, then the ultrasonic interrogation signal may experience partial or complete attenuation or scattering due to tissue structures such as ribs, lungs, combinations thereof, and the like. Additionally, or alternatively, a first device may have moved in space and/or rotated relative to a previous position and/or orientation. Conventional imaging or beamforming techniques to scan a predetermined tissue volume and locate a first device may be time-consuming, and may further require high power, increased design complexity, and/or expertise for execution. For example, conventional ultrasonic beamforming techniques comprise a phased array configured to scan the surroundings of a first device may be time-consuming since such approaches may use a small focal spot size of the beam (e.g., ˜millimeter dimensions) to scan a large region (e.g., several centimeters in all three dimensions) in search of a miniature IMD.

i. Broad Beam Interrogation

In some variations, an interrogation signal may comprise a broad ultrasound beam with a wide beam diameter (e.g., a spatially unfocused beam, a plane wave or an approximate plane wave). In some variations, the first device may comprise an ultrasound transducer and a diameter (e.g., a half-power beam diameter) of the broad ultrasound beam, at the depth of the first device in tissue, may be greater than a dimension of the ultrasound transducer (e.g., greater than about four times the maximum lateral dimension of the first device's ultrasound transducer). As an example, in some variations, the half-power beam diameter of an interrogation signal may be about 10 cm, while the transducer of a first device may have a width of about 1 mm in each dimension. Such a design may be useful for spanning a large tissue volume with the interrogation signal and, thus, maximizing the chances of the interrogation signal being detected by the first device.

In some variations, an interrogation signal may be transmitted by a second device using a sub-array comprising one or more ultrasonic transducer elements, as described in detail herein. In some variations, one or more ultrasonic transducer elements may be configured for additional operations such as receiving one or more feedback signals, transmitting power, data, commands or other signals to a first device, receiving data, commands or other signals from a first device, combinations thereof, and the like. In some variations, it may be advantageous to design separate ultrasonic transducer elements for the purposes of transmitting an interrogation signal and for transmitting power to a first device. For example, an external wireless device may comprise two separate transducer arrays, where one or more elements of a first array may be configured to transmit an interrogation signal, and one or more elements of a second array may be used to transmit power. In some variations, the two separate arrays may comprise transducer elements of different dimensions and/or different materials.

One variation of an ultrasonic transducer element is described herein for generating a broad beam for interrogation of a first device. Example calculations presented herein use a circular disc ultrasonic transducer element as an example. Similar calculations may be performed for other transducer shapes (e.g., square, rectangular cross-sections, and the like). For example, equations known in the art for other transducer shapes (e.g., with a square cross-section) may be used instead of the equations for a circular disc presented herein. Transducer element design presented herein may be complemented with a design for other transducer parameters, including one or more of transducer material, thickness, spacing between elements, combinations thereof, and the like.

Consider a first device comprising an ultrasonic transducer configured to receive an ultrasonic interrogation signal from the external wireless device. In some variations, an interrogation signal may have a low frequency (e.g., 100 kHz, 200 kHz, and the like) configured to enable interrogation with a broad beam diameter since a low frequency corresponds to a large wavelength. In some variations, the interrogation signal's frequency may be at or close to one or more resonance frequencies (e.g., open-circuit resonance frequency, short-circuit resonance frequency, harmonics of a resonance frequency, etc.) of the ultrasonic transducer of the first device. At or close to such a frequency, the impedance of the ultrasonic transducer may be real, or approximately real, and may be denoted by R_{P}. In some variations, the interrogation signal frequency may be an off-resonance frequency for the ultrasonic transducer, where the ultrasonic transducer impedance may be complex. As an example, a miniature ultrasonic transducer (e.g., mm-sized) may be designed such that its R_{P} may be between about 0.5 kΩ and about 500 kΩ at one or more of its resonance frequencies. For the successful detection of the interrogation signal by the first device, the open-circuit voltage (V_{OC}) produced at the first device ultrasonic transducer, due to the interrogation signal, may need to be greater than or equal to a predetermined detection threshold. For example, a first device may be configured to use a V_{OC} detection threshold of about 0.2 V (peak voltage). In some variations, such a low detection threshold may be possible if a first device comprises stored energy, e.g., a battery, for providing energy for its operation and/or for the generation of a feedback signal. In some variations where a first device may not have sufficient stored energy (e.g., no battery), the first device may be configured to use a higher detection threshold (e.g., about 0.5 V) in order to overcome the threshold voltage of typical rectifier circuits, and harvest energy for the generation of a feedback signal. Setting a detection threshold for the V_{OC} of an ultrasonic transducer of the first device is meant to serve as an example here. In some variations, a detection threshold may be set for a DC voltage generated upon rectification of the interrogation signal, envelope of the interrogation signal, power/energy received by the first device through the interrogation signal, duration of the interrogation signal, data encoded in the interrogation signal (e.g., a code or a unique ID), combinations thereof, and the like.

The required available electrical power (P_{AV}) at the ultrasonic transducer of the first device may be given by:

$${P}_{AV} = \frac{{V}_{OC}^{2}}{8 {R}_{P}} \qquad ( 1 )$$

Thus, for V_{OC} of 0.2 V (peak voltage) and R_{P} in the range of about 0.5 kΩ to about 500 kΩ, the required P_{AV} may be between about 10 nW to about 10 μW. Further, it may be assumed that the ultrasonic transducer of the first device may have an area, denoted by A, of about 1 mm², as an example, and an aperture efficiency (or acoustic-to-electrical power conversion efficiency), denoted by ηₐₚ, of about 0.5, as an example. The required acoustic intensity at the ultrasonic transducer of the first device (I_{wm}) may be given by:

$${I}_{wm} = \frac{{P}_{AV}}{A \times {η}_{ap}} \qquad ( 2 )$$

Thus, the required I_{wm} may be between about 0.02 μW/mm² to about 20 μW/mm². A sub-array of the second device may generate I_{wm} at the location of the first device that may be higher than such an estimated minimum required intensity.

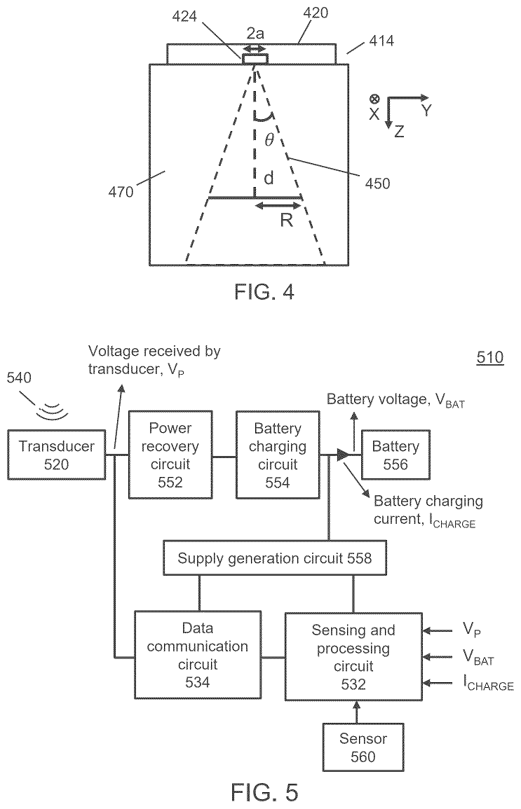

Next, consider a circular disc ultrasonic transducer element (424) with radius ‘a,’ which may be included in the second device, as shown in FIG. 4. This transducer element (424) may transmit an interrogation signal to a first device (not shown). FIG. 4 shows a schematic representation of an ultrasonic beam (450) transmitted by the transducer element. The radius of this ultrasonic beam (450) in the X or Y directions (e.g., the half-power beam radius or the radius at which the acoustic intensity may fall 3 dB below the intensity at the center of the beam), at a tissue depth of ‘d’ in the Z direction, may be denoted by ‘R.’ The corresponding half-power beam angle, θ, as shown in FIG. 4, may be given by:

$$θ = {tan}^{- 1} ( \frac{R}{d} ) \qquad ( 3 )$$

The radius ‘a’ may be related to the half-power beam angle ‘θ’ by:

$$ka sin θ = 1.6 \qquad ( 4 )$$

Here, k is an angular wavenumber related to the wavelength (λ), frequency (f) and speed (c) of the ultrasonic wave in tissue by:

$$k = \frac{2 π}{λ} = \frac{2 π f}{c} \qquad ( 5 )$$

For a predetermined choice of d, R and f, an estimate of the required element radius may be obtained. As an example, for a tissue depth (d) of 5 cm, a beam radius (R) at this tissue depth of 7.5 cm (i.e., a beam diameter of 15 cm), and an ultrasonic frequency (f) of 0.5 MHz, the transducer element radius (a) may be computed to be about 0.92 mm (i.e., element width or diameter of about 1.84 mm), based on the equations. In this example, the value of ka may be about 1.92. For this element size, the half-power beam angle θ may be about 56.3°, and the beam diameter at any tissue depth may be about 3 times the tissue depth (assuming a homogeneous tissue medium for simplicity). Thus, for a tissue depth of about 20 cm, the beam diameter may be about 60 cm, which may be sufficiently large for interrogation of a first device located at this depth. As another example, if at a tissue depth of 20 cm, a beam radius of 7.5 cm is desired, then the above equations may be configured to estimate a transducer element radius (a) of about 2.18 mm (i.e., element width or diameter of about 4.35 mm). A homogeneous tissue medium is assumed here for simplicity. Presence of heterogeneous tissue layers and/or structures such as ribs/lungs may be included in more advanced calculations and/or simulations, without fundamentally changing the analysis presented herein.

Next, it may be assumed that the transducer element included in the external wireless device transmits a power denoted by P_{TX}. The acoustic intensity at depth d at the center of the beam (i.e., on the axis of the transducer element), denoted by I₀, may be given by:

$${I}_{0} = \frac{{P}_{TX}}{4 π {d}^{2}} \times {D}_{f} \times 1 {0}^{- \frac{{α}_{dB} \times d \times f}{10}} \qquad ( 6 )$$

Here, D_{f} denotes a directivity function of the transducer element, which may represent an amount by which the transducer element may focus an ultrasonic beam relative to a uniform omni-directional transmitter. The tissue attenuation coefficient of ultrasound in dB is denoted by α_{dB}. For example, α_{dB} may have a value of 1 dB/(cm·MHz) for soft tissue, resulting in a total attenuation for a 5 cm tissue depth and 1 MHz frequency of about 5 dB.

The directivity function may be written in terms of ka, and may be given by:

$${D}_{f} = \frac{{( ka )}^{2}}{1 - \frac{{J}_{1} ( 2 ka )}{ka}} \qquad ( 7 )$$

Here, J₁ denotes an order-one Bessel function of the first kind. For example, for a ka value of about 1.92 estimated above, the value of D_{f} may be about 3.69.

The acoustic intensity at the half-power beam radius, R, may be given by (I₀/2). Thus, if a first device is located anywhere on or inside the half-power beam radius, it may receive an acoustic intensity greater than or equal to (I₀/2). In order to achieve (I₀/2) greater than or equal to the required acoustic intensity, I_{wm}, estimated above, the required P_{TX} may be given by:

$${P}_{TX} \geq \frac{{I}_{wm} \times 2 \times 4 π {d}^{2} \times 1 {0}^{+ \frac{{α}_{dB} df}{10}}}{{D}_{f}} \qquad ( 8 )$$

As an example, using the above equation, and values considered in the above example, for achieving an acoustic intensity greater than or equal to 0.02 μW/mm² (as estimated above for R_{P} of 500 kΩ) within a beam radius of about 7.5 cm at about 5 cm tissue depth, the required P_{TX} at the transducer element of the external wireless device may be about 0.6 mW. As another example, for a tissue depth of about 20 cm and a beam radius of about 30 cm (for which the required transducer element size may be the same, i.e., element radius of about 0.92 mm, as described above), the required P_{TX} may be about 54.5 mW. As another example, for a tissue depth of about 20 cm and a beam radius of about 7.5 cm, the required P_{TX} may be about 9.2 mW.

Thus, as shown in the above example, transducer element size (e.g., element radius a) and minimum required transmit power (P_{TX}) may be estimated for scanning a predetermined region in tissue such that a feedback signal may be generated from a first device, if the first device is disposed in the corresponding region in tissue (e.g., within the beam radius at a predetermined tissue depth).

In some variations, a high P_{TX} may be used as allowed by safety limits for the body. Configuring the second device to use approximately the highest allowed transmit power or intensity, or a fraction thereof (e.g., half or ⅕^{th} of the maximum allowed transmit power or intensity), for interrogation of a first device, may help with maximizing the chances of the first device detecting the interrogation signal without causing any harm to body tissue. Such one or more transmit power or intensity levels for an interrogation signal may be predetermined and hardcoded in the processor of the external second device, or may be determined dynamically through real-time feedback of tissue temperature or tissue heating, and the like. In some variations, the interrogation signal may encode a unique identification (ID) number or command as described herein.

In some variations, an RF or magnetic interrogation signal may be used, instead of or in addition to an ultrasonic interrogation signal, where the transducer of the external wireless device and the first device may comprise one or more coils or antennas for transmitting and/or receiving such an RF or magnetic interrogation signal. An advantage of using an RF or magnetic interrogation signal may be that the energy of the interrogation signal may be spread over a large tissue volume (due to a large wavelength). Additionally, an RF or magnetic interrogation signal may not experience significant attenuation due to the rib cage or lungs. The frequency of such an RF or magnetic interrogation signal may be between about 100 kHz to about 10 GHz.

ii. Interrogation Frequency

In some variations, the interrogation signal may comprise a first frequency and one or more of the wireless power and data may comprise a second frequency different than the first frequency. In some variations, the first device (e.g., IMD) may comprise at least one ultrasound transducer comprising a first impedance corresponding to the first frequency and a second impedance corresponding to the second frequency. The first impedance may be greater than the second impedance. In some variations, the first device may comprise a first ultrasound transducer comprising a first impedance corresponding to the first frequency, and a second ultrasound transducer comprising a second impedance corresponding to the second frequency. The first impedance may be greater than the second impedance. This may allow generation of a high voltage at the first device's ultrasound transducer for a predetermined intensity of the interrogation signal. For example, for a predetermined acoustic intensity at the first device (I_{wm}), and a fixed area (A) and aperture efficiency (ηₐₚ) of the first device's ultrasound transducer, a high R_{P} will result in a large V_{OC} (see Equations (1) and (2). This may be advantageous if a first device is configured to have a certain voltage detection threshold (e.g., a minimum V_{OC} or a DC voltage generated upon rectification of the interrogation signal, etc.).

One variation of interrogation frequency selection is described herein. A first device may comprise a millimeter (mm) or sub-mm sized piezoelectric. The transducer may comprise a short-circuit resonance frequency (f_{SC}) of about 1 MHz with an impedance at short-circuit resonance (R_{SC}) of about 2 kΩ, and an open-circuit resonance frequency (f_{OC}) of about 1.3 MHz with an impedance at open-circuit resonance (R_{OC}) of about 200 kΩ. As discussed herein, a first device may be configured to have a V_{OC} detection threshold of about 0.2 V (peak voltage) for detecting an interrogation signal. If the interrogation signal has a frequency of about 1 MHz (close to f_{SC}), based on equation (1) above, the minimum required P_{AV} to overcome the V_{OC} detection threshold may be about 2.5 μW (since R_{SC} is about 2 kΩ). Similarly, if the interrogation signal has a frequency of about 1.3 MHz (close to f_{OC}), the minimum required P_{AV} to overcome the V_{OC} detection threshold may be about 0.025 μW (since R_{SC} is about 200 kΩ), which is about 100 times lower than the required P_{AV} at about 1 MHz. Consequently, the required intensity of the interrogation signal at the first device, and the required power (P_{TX}) that the external wireless device may need to transmit, may be about 100 times lower in this example, which may reduce the energy consumption of the external wireless device and reduce unnecessary tissue heating. Thus, in this variation, it may be advantageous to select a frequency close to or equal to f_{OC} for interrogation of the first device. While the calculations above are shown at f_{SC} and f_{OC}, and specific values of these frequencies and impedance are presented as examples, the concept applies generally, and highlights the advantage of selecting any frequency (not necessarily the resonance frequency) where the real part of the first device's transducer's impedance may be high.

In some variations, while the interrogation signal may use a frequency at which the first device's transducer may have a high impedance (for reliable detection of the interrogation signal), power transfer may be performed at a different frequency. This may be because the constraints for efficient power transfer may be different from the constraints for reliable detection of an interrogation signal. For example, in the example presented above, while the interrogation signal may use a frequency close to f_{OC} due to the advantage in generating a high V_{OC}, wireless power transfer from the external wireless device to the first device may be performed at f_{SC}, which may be advantageous in terms of lower tissue losses and better impedance matching between the transducer's impedance and the electrical load of the first device.

In some variations, a transducer of a first device may comprise a plurality of transducer elements, where all transducer elements may not have the same frequency or frequency range where their impedance may be sufficiently high for allowing interrogation with a low power. In such variations, the external wireless device may interrogate the first device at different frequencies (e.g., one after the other or simultaneously) in order to allow the first device to successfully detect the interrogation signal.

iii. Reliable Interrogation

In some variations, a second device may not detect any feedback signal from a first device in response to its transmitted interrogation signal. This may be due to one or more reasons including, but not limited to, the first device is outside of the interrogation signal's beam (e.g., even when interrogation is performed using a broad beam), the interrogation signal is partially or completely attenuated or scattered by tissue structures such as ribs, lungs, etc., the first device is temporarily inaccessible to the interrogation signal (e.g., IMD moved to a location behind a lung during a part of the cardiac or breathing cycle), the first device rotated significantly, and the like.

In some variations, a system configured to exchange power or data may comprise a first device (e.g., an IMD) and a second device (e.g., a wireless device) comprising a processor and a transducer array. The transducer array may comprise a plurality of sub-arrays, where a first sub-array may be configured to transmit an interrogation signal to the first device, a second sub-array may be configured to receive a feedback signal from the first device, and where the processor may be configured to cycle through one or more sub-arrays of the plurality of sub-arrays after transmitting the interrogation signal until the received feedback signal satisfies a predetermined condition. For example, in some variations, the predetermined condition may compare a strength of the received feedback signal to a threshold. In some variations, an absolute strength of a received feedback signal at one or more transducer elements may be compared to a predetermined threshold. In some variations, a relative strength of the received feedback signal between two or more transducer elements may be compared (e.g., a difference between the strength of the received feedback signal across two or more transducer elements). The processor may be configured to cycle through one or more sub-arrays for transmitting the interrogation signal if the feedback signal strength is below the threshold.

As an example, a second device may comprise more than one transducer element for transmitting an interrogation signal. If a feedback signal does not satisfy a predetermined condition, the external wireless device may transmit an interrogation signal through a second transducer element, and so on. For example, in some variations, an external wireless device may comprise a transducer element in the center and one or more transducer elements along its periphery. It may first transmit an interrogation signal through a center element, and, if no feedback signal is received, it may then transmit an interrogation signal through an element near its periphery. The external wireless device may cycle through a plurality of transducer elements one by one, in a predetermined order, to transmit interrogation signals until a feedback signal is received from a first device. Such cycling through a plurality of transducer elements may be performed once or more than once. An advantage of cycling through elements in a predetermined order may be simplicity (low complexity) in the implementation of the external wireless device. An algorithm for cycling through a plurality of transducer elements for the interrogation of a first device may be implemented in the processor of the external wireless device (e.g., a binary search algorithm).

In some variations, the external wireless device may be configured to operate in the receive mode for a predetermined time duration, after transmitting an interrogation signal, in anticipation of the feedback signal. For example, in some variations, such a time duration may be between about 50 μs and about 1 ms. This time duration may be determined based on the round-trip travel time of a signal between the external wireless device and the first device, and any wait time that may be implemented on the first device between the receipt of an interrogation signal and the transmission of a feedback signal. As an example, the external wireless device may cycle through a plurality of transducer elements one by one after every about 1 ms until a received feedback signal satisfies a predetermined condition. Such a rapid interrogation scheme may help elicit a feedback signal from a first device quickly (e.g., within a few seconds) even if it is temporarily blocked by tissue structures such as ribs or lungs, because the natural motion of the first device due to heart motion or breathing may be slow (e.g., period on the order of a second).

In some variations, the external wireless device may provide a user prompt corresponding to the status of interrogation, and whether a feedback signal from the first device is received or not. A user prompt may be helpful for manual adjustment or repositioning of the external wireless device. Different variations of user prompt or feedback, as discussed above, are applicable herein. For example, if no feedback signal is received upon transmission of an interrogation signal from one or more transducer elements, the external wireless device may notify a user, via visual and/or audio notifications, to move the external wireless device on a patient's chest (e.g., move towards the left shoulder). In some variations, if no feedback signal is received, a user may be asked to move the external wireless device to different predetermined locations on a patient's chest (e.g., while displaying a realistic or stylized image of the chest to instruct the user).

In some variations, an external wireless device may be configured to process reflections of an interrogation signal or perform imaging. An interrogation signal may experience reflections at one or more of skin, one or more ribs, lung, combinations thereof, and the like. Processing the reflection of an interrogation signal, or imaging, may be useful to determine the next transducer element for transmitting an interrogation signal if no feedback signal is received in response to a currently transmitted interrogation signal.

In some variations, an external wireless device may determine a time window for transmitting an interrogation signal based on one or more physiological parameters that it may be configured to measure, including but not limited to parameters such as heart rate, breathing rate, blood pressure, heart sounds, combinations thereof, and the like. This may be advantageous in a scenario where a first device may be temporarily blocked by tissue structures such as ribs or lungs during a part of the cardiac or breathing cycle.

In some variations, if no feedback signal is detected by an external wireless device in response to an interrogation signal transmitted via a first transducer element, the external wireless device may be configured to modify one or more parameters of the interrogation signal including, but not limited to, frequency, amplitude, duration, phase, time delay, combinations thereof, and the like, and re-transmit an interrogation signal via the same or a different transducer element.

In some variations, any subset or a combination of techniques, or a combination of any subsets of the techniques presented above, may be used. Such techniques may be applied in any feasible order until a feedback signal satisfies a predetermined condition. For example, in some variations, an external wireless device may be configured to first cycle through a plurality of transducer elements with a predetermined set of interrogation signal parameters (e.g., a fixed frequency, amplitude, duration, and the like), then optionally attempt a modification of one or more parameters of the interrogation signal (e.g., frequency, amplitude, duration, and the like), and then provide user prompt for manual adjustment of the external wireless device, and optionally repeat this process, until a feedback signal is detected.

iv. Feedback Signal

In some variations, a feedback signal may be transmitted by a first device (e.g., IMD) in response to receiving an interrogation signal. However, reception of the feedback signal by a second device (e.g., external wireless device, wireless device) may be inconsistent due to one or more factors including, but not limited to rotation of the first device, suboptimal radiation pattern of a transducer of the first device, feedback signal attenuation or scattering in the link, interference between the feedback signal and reflection(s) of the interrogation signal received by the second device, combinations thereof, and the like. Solutions provided herein may be useful to overcome such challenges.

In some variations, a transducer of a first device may comprise more than one transducer element configured to enable an aggregate radiation pattern with a wide acceptance angle as described herein. If an uplink signal is transmitted by a first device simultaneously using more than one transducer elements, the resulting waves may undergo interference that may lead to partial or complete signal cancellation (e.g., null lobes). In some variations, selecting one transducer element for transmitting an uplink signal (e.g., a feedback signal) may reliably propagate the uplink signal to the second device. In some variations, such a transducer element may be selected based on the interrogation signal power and/or voltage received by the one or more transducer elements of the first device. For example, a processor of the first device may process the interrogation signals received by different transducer elements, and then compare their respective signal amplitudes, power, and/or voltage (e.g., amplitude of V_{OC}, or a rectified DC voltage generated from each transducer element). The processor may then select the transducer element that received the highest power and/or voltage, or a power and/or voltage higher than a predetermined threshold, as the transducer element to be used for transmitting the feedback signal. The transducer element identified as receiving the highest power and/or voltage of the interrogation signal may comprise the highest link gain among the transducer elements, or the most favorable radiation pattern, for exchanging signals (e.g., power, data) with the second device (based on reciprocity). By transmitting a predetermined power signal through the transducer element with the highest link gain, instead of distributing that predetermined power signal among several transducer elements (some of which may not have a sufficient link gain with the second device), the overall link gain and corresponding signal-to-noise ratio (SNR) of the uplink signal at the second device may be maximized. Furthermore, energy consumption of the first device may be minimized by selective transducer element selection, thereby extending a battery life for a battery powered IMD. In some variations, the second device may program the first device (via a downlink command, a command encoded in the interrogation signal, and the like) to configure a particular transducer element for transmission of an uplink signal (e.g., a feedback signal).

In some variations, a first device may be configured to transmit an uplink signal (e.g., a feedback signal) with a power level that may be sufficient to result in a minimum required SNR at the second device for reliable detection and/or decoding of the uplink signal. In some variations, the first device may be configured to transmit an uplink signal with a first power (e.g., greater than required to meet the SNR requirement) that may be limited by one or more of predetermined safety limits of the body, a predetermined voltage limit of the transmitter circuit (e.g., set by a breakdown voltage of an integrated circuit), a limit based on a predetermined energy budget of the first device, combinations thereof, and the like. Transmitting an uplink signal with a higher power level may enable the second device to reliably receive the feedback signal or any uplink signal in spite of tissue-based losses, scattering due to tissue structures, relative movement and/or rotation between the first device and the second device, combinations thereof, and the like. In some variations, the power level transmitted by a first device for an uplink signal may be determined by the first device based on the power or voltage received by one or more of its transducer elements due to an interrogation signal, since such a power or voltage may be a surrogate for, or may be used to estimate the link gain. In some variations, the first device may be programmed by the second device via a downlink signal (e.g., a command) to transmit a predetermined power level for an uplink signal, which may be based on an estimation of the link gain by the second device based on a received feedback signal from the first device.

In some variations, an interrogation signal may reflect from tissue boundaries, ribs, lungs, and combinations thereof. A feedback signal transmitted by a first device may interfere with such reflections of the interrogation signal. Additionally or alternatively, the second device may not have precise knowledge of a location of first device, including a separation distance between the first device and the second device. Conventionally, a second device may not know the time or time window in which a feedback signal may arrive, and the second device may be unable to distinguish whether a received signal is a reflection of the interrogation signal or a feedback signal.

In some variations, interference of a received signal may be reduced by configuring a first device to wait for a predetermined time delay (e.g., greater than about 10 μs) between the receipt of an interrogation signal and transmission of a feedback signal. For example, a second device may be configured to interrogate (using ultrasound signals) a first device that may be located at a tissue depth or separation up to a maximum separation of about 20 cm, where the second device may not know the separation distance a priori. If any tissue structure or tissue boundary is located within this maximum separation distance from the second device, reflections from such a structure or boundary may reach the second device at up to a time of about 267 μs (assuming a maximum separation of about 20 cm and speed of sound in tissue of about 1500 m/s) after its transmission of the interrogation signal. Accordingly, a first device may be configured to wait for a time delay of at least about 267 μs between the receipt of the interrogation signal and transmission of the feedback signal. In some variations, the time delay may be selected based on a predetermined tissue depth at which the first device may be assumed to be located. Such a technique may allow enough time for potential reflections of the interrogation signal to dissipate or die down sufficiently so as to not interfere with the feedback signal.

In some variations, the first device may comprise a timer (e.g., wait timer) comprising one or more circuits or techniques including, but not limited to, a relaxation oscillator, RC oscillator, ring oscillator, capacitive charging or discharging, frequency-locked loops, combinations thereof, and the like. In some variations, the circuits may be designed for low power consumption. For example, ultra-low power relaxation oscillators may be configured to generate a time delay of about hundreds of μs and even up to several ms, if needed, with energy consumption that may be well below the stored energy of the first device (e.g., energy stored on a miniature battery). In some variations, the second device may be configured to receive a feedback signal after the time delay that the first device is set for. In this manner, the second device may be able to distinguish between tissue reflections and the feedback signal.

In some variations, the feedback signal and reflections from the interrogation signal may be distinguished by configuring the systems described herein with different wireless modalities (e.g., modulations) between the feedback signal and the interrogation signal. For example, the interrogation signal may comprise an RF or magnetic signal while the feedback signal may comprise an ultrasonic or acoustic signal, or vice versa.

In some variations, the feedback signal may be distinguished from reflections of the interrogation signal by configuring the wireless system with different frequencies for the feedback signal and the interrogation signal. For example, the interrogation signal may comprise a frequency close to f_{OC} (e.g., about 1.3 MHz as discussed herein) of a transducer of a first device. The feedback signal may comprise a frequency close to f_{SC} (e.g., about 1 MHz as discussed herein). As another example, in some variations, an interrogation signal may comprise a relatively lower frequency (e.g., 100 kHz, 200 kHz, and the like) to enable interrogation with a broad beam diameter (due to large wavelength). Power transfer (and/or transfer of downlink signals) may be performed at a relatively higher frequency (e.g., 1 MHz) to allow focusing of the beam at the first device with an approximately millimeter beam diameter for higher power transfer efficiency. The reflections of the interrogation signal may comprise the frequency of the interrogation signal, thereby allowing the feedback signal to be distinguished from an interrogation signal.

In some variations, a feedback signal may be distinguished from an interrogation signal based on data contained within the feedback signal. In some variations, the feedback signal may comprise one or more of a code, unique ID, unique waveform feature (e.g., data bits with a particular modulation scheme), combinations thereof, and the like, that may be compared to reflections of an interrogation signal. In such variations, a processor of the second device may process received signals to identify and distinguish a feedback signal from reflections of the interrogation signal. In some variations, signal processing techniques may comprise a matched filter configured to detect the presence of a code or template, or more generally, a feature that may only be present in a feedback signal and not in any other signals such as reflections of an interrogation signal. In some variations, the processor may perform this processing in real-time.

In some variations, transmission of a feedback signal with a relatively higher power (which may still be below safety limits) may itself be sufficient to be distinguishable from reflections of an interrogation signal since the reflections may have a relatively lower power due to tissue attenuation, imperfect reflections, and scattering from tissue structures. Any subset or a combination of techniques, or a combination of any subsets of the techniques presented above may be used together as described herein.

v. Interrogation Signal Detection

In some variations, reliable detection of a downlink signal such as an interrogation signal, downlink data, and the like, by a first device may allow a wireless link to be efficiently established and maintained. In some variations, interrogation signals may comprise a downlink signal such as downlink data or commands transmitted by the second device to one or more IMDs. In some variations, a second device may be configured to transmit an interrogation signal at a frequency at which a transducer of a first device transducer may comprise a relatively high impedance. The high impedance may enable generation of a relatively large voltage at the terminal of a transducer for a predetermined power, thereby increasing the sensitivity of the first device and enabling detection of low-power interrogation signals. For example, the interrogation signal may comprise a frequency equal to an open-circuit resonance frequency (f_{OC}) of an ultrasound transducer of the first device. Other signals such as power signal and/or data signal may be transmitted to the first device at a different frequency such as a short-circuit resonance frequency (f_{SC}), or a frequency in the inductive band of the ultrasound transducer (i.e., a frequency between f_{SC} and f_{OC}). Additionally or alternatively, in some variations, the first device may comprise one or more impedance transformation networks coupled to one or more ultrasound transducers of the first device. The impedance transformation network may transform the impedance of the ultrasound transducer or its received voltage to a higher value. For example, an impedance transformation network may comprise a capacitive network. The first device may comprise an envelope detector circuit and a comparator circuit configured to compare the received envelope with a predetermined threshold voltage (e.g., a fixed reference voltage).

In some variations, a first device may comprise a first ultrasound transducer configured to detect an interrogation signal and/or downlink data, and a second ultrasound transducer configured to receive power. The first, second, or a third ultrasound transducer may be used for transmitting uplink data. For example, the first ultrasound transducer configured to detect an interrogation signal may comprise one or more of a piezoelectric transducer, a capacitive micromachined ultrasound transducer (CMUT), and the like. The first ultrasound transducer may comprise a high impedance at the interrogation or downlink data frequency, and/or may be coupled to an impedance transformation network to up-transform its impedance and received voltage. The second ultrasound transducer may be configured independently for high-efficiency power recovery and/or data transmission.

In some variations, uplink data transmission using an ultrasound transducer of a first device may be performed at f_{OC} of the ultrasound transducer of the first device. Power may be transmitted by the second device at f_{SC} of the ultrasound transducer of the first transducer. Receiving power at f_{SC} may enable an efficient impedance match between the ultrasound transducer of the first device and power recovery circuits, thus providing a higher overall power recovery efficiency. In some variations, a first device may transmit uplink data at f_{OC} of the ultrasound transducer element(s) of the second device since the transducer element(s) may have a higher impedance at that frequency. Accordingly, the ultrasound transducer element(s) of the second device may be configured to generate a higher voltage for a predetermined received power level of the uplink data, thereby enabling reliable detection of uplink data.

b. Transducer Configuration Selection

In some variations, a transducer configuration may be selected based on received feedback signal. For example, FIGS. 3A, 3B, and 3C describe transducer configuration (328) selection based on a feedback signal (352). In some variations, a first device (e.g., IMD) may be configured to transmit a feedback signal to a second device (e.g., wireless device) upon receiving an interrogation signal. In some variations, the feedback signal may comprise one or more of an analog pulse, acknowledgment signal, a digital first device energy state, digital interrogation signal strength, identification number, code, command, one or more parameters of the first device, wireless power signal, and data signal. In some variations, the feedback signal may comprise one or more of ultrasonic reflection signals and ultrasonic backscatter signals corresponding to the interrogation signal as described herein. In some variations, the first device may be configured to modulate the ultrasonic backscatter signal. In some variations, the first device may be configured to transmit a feedback signal at one or more frequencies. In some variations, the processor of the second device may be configured to identify a frequency of the transducer configuration for transmitting one or more of wireless power and downlink data to the first device based on the feedback signal. In some variations, the identified frequency of the transducer configuration may correspond to the feedback signal frequency at a maximum amplitude. The identified frequency may correspond to an optimal power frequency for each patient since different patients may have different tissue depths and tissue composition (e.g., fat content, rib structure, and the like). In some variations, the feedback signal may comprise transmission of one or more analog and digital feedback signals.

In some variations, the feedback signal may include, but is not limited to, a signal acknowledging the receipt of the interrogation signal, an analog feedback signal (e.g., one or more ultrasonic pulses comprising one or more cycles of a carrier frequency), data encoded using any modulation technique (e.g., digital modulation), combinations thereof, and the like. In some variations, a first device may not explicitly transmit a different signal for acknowledging the receipt of the interrogation signal, but the transmission of an analog feedback signal and/or data may itself serve as acknowledgment of the receipt of the interrogation signal. In some variations, a first device may be configured to transmit such components of the feedback signals in any order. For example, a first device may be configured to first transmit digital data bits, followed by an analog feedback signal, or vice versa.

In some variations, selecting one or more of the transducer configurations of the second device may comprise estimating a strength of the feedback signal received by the second sub-array of the second device, and exchanging one or more of the wireless power signal and data signal using the one or more transducer configurations based on the estimated strength of the received feedback signal. In some variations, a processor of the second device may be configured to process the analog feedback signal to generate feedback signal data comprising a power and/or voltage amplitude of the feedback signal received by transducer elements of the second device. The processor may be configured to select a transducer configuration of the second device based on comparison of the received strength of the feedback signal to a predetermined threshold. For example, transducer elements configured to receive a feedback signal power and/or voltage amplitude above a predetermined threshold may be selected as the transducer configuration for exchanging power and/or data with the first device. A predetermined threshold may comprise an absolute threshold, relative threshold, or adjustable threshold. For example, in some variations, if the maximum feedback signal amplitude received among all transducer elements of the RTC is A₀, then only transducer elements receiving an amplitude greater than or equal to A₀/3 (or A₀/\sqrt{2}, and the like) may be selected to comprise the selected transducer configuration.

In some variations, selective powering of the transducer elements may improve one or more of energy efficiency, tissue heating, and link efficiency. In some variations, only transducer elements comprising the selected transducer configuration may be powered ON during operation (e.g., transmission, reception), while non-selected transducer elements may be turned OFF. For example, tissue structures such as ribs, lungs, and the like, may attenuate or obstruct an ultrasonic feedback signal received by one or more transducer elements of the second device, resulting in a lower received power and/or voltage at such transducer elements compared to other transducer elements where the feedback signal is not attenuated or obstructed by such tissue structures. Based on reciprocity, a signal transmitted through an obstructed set of transducer elements may be attenuated by tissue structures and result in lower received power at the first device. Additionally, it may result in unnecessary heating at or near such tissue structures. Thus, it may be beneficial to not transmit power through such transducer elements, thereby, minimizing unnecessary tissue heating, conserving energy of the second device, and achieving an overall high link efficiency (e.g., where link efficiency may be defined as the total available power at the first device divided by the total power transmitted by the second device). As shown in FIG. 3C, one or more transducer elements may be blocked by ribs and/or transducer elements such that the blocked transducer elements may receive low power from the feedback signal (e.g., transducer elements that may be much farther away from the first device compared to other transducer elements, and may, thus, experience more propagation loss through tissue). The blocked transducer elements may not comprise the selected transducer configuration.

In some variations, the processor of the second device may be configured to process the feedback signals received by at least the transducer elements comprising the selected transducer configuration to generate feedback signal data comprising one or more parameters including frequency, phase (and/or time delay) and amplitude of the feedback signal received by each of those transducer elements, combinations thereof, and the like. Determination of these parameters may be useful for determining the driving signals for each of the transducer elements comprising the selected transducer configuration using techniques such as time reversal or beamforming. Time reversal may comprising driving a set of transducer elements with a phase or a delay that may be opposite or reversed with respect to the phase or delay of the received feedback signals at the set of transducer elements.

In some variations, the frequency of a feedback signal (or any uplink signal in general) of a first device may be configured by an oscillator circuit coupled to the processor of the first device. The frequency may not be precisely known to a second device a priori. For example, the frequency of several IMDs may lie on a distribution (e.g., a Gaussian distribution with a mean of about 1 MHz and a standard deviation of about 50 kHz, or about 5% of the mean) based on effects that may be typical in integrated circuit fabrication such as process variations, circuit mismatch, combinations thereof, and the like. The second device may be configured to drive the corresponding transducer elements of a selected transducer configuration with reversed phases based on the phases of the received feedback signal using a frequency (e.g., 1 MHz) different from the frequency of the feedback signal (e.g., 1.15 MHz or three standard deviations higher than mean). The waves generated by the transducer elements may constructively interfere at a location different from the first device's location, resulting in sub-optimal or no focusing of energy at a location of the first device.

In some variations, the second device may be configured to identify a frequency of the received feedback signal and may use the same frequency for powering the first device, along with reversed phases or delays based on the phases or delays of the received feedback signal. In some variations, the second device may be configured to wirelessly power the first device at a frequency being a scaled version of the frequency of the received feedback signal. For example, the frequency of the received feedback signal may be multiplied or divided by a scaling factor (e.g., an integer scaling factor). In some variations, one or more transducer configurations selected for transmitting power and/or data to the first device may comprise a frequency different from the identified frequency.

Additionally or alternatively, the processor of the second device may generate feedback signal data for estimating a location or a set of spatial coordinates of the first device in tissue relative to the second device based on the received analog feedback signal. For example, the processor may be configured to perform triangulation based on the relative arrival times or time of flight of the feedback signal at three or more transducer elements of the second device. In some variations, the processor may be configured to process relative arrival times of the feedback signal for less than three transducer elements (or less than three spatial coordinates) to estimate an approximate location of the first device. In some variations, an estimated location of the first device determined using triangulation may be further used to determine the appropriate driving signals for the transducer elements of the selected transducer configuration. For example, estimation of the location or a set of spatial coordinates of the first device relative to the second device may allow determination of the phase that each transducer element of the selected transducer configuration may be driven with for any frequency of wireless powering (e.g., about 1 MHz) different from the frequency of the feedback signal (e.g., about 1.15 MHz).

In some variations, focusing of energy at the first device may correspond to a focal spot size larger than the dimensions of one or more transducers of the first device. Focusing energy may reduce sensitivity of received power due to one or more of inaccuracies in focusing, small relative movements or rotations of the first device, other link aberrations, combinations thereof, and the like. For example, the driving signals of the selected transducer configuration (such as phases) may be adjusted to achieve an ultrasonic focal spot diameter that may be about two times the width of an ultrasonic transducer of first device configured to receive wireless power.

In some variations, the processor of the second device may generate feedback signal data comprising a power and/or voltage amplitude of the feedback signal received by each of the transducer elements of the second device or the selected transducer configuration. The feedback signal data may be generated based on the received analog feedback signal. The feedback signal data may be used to estimate a link gain and determine the power to be transmitted through each transducer element of the selected transducer configuration to provide a predetermined received power at the first device.

In some variations, a downlink-based sub-array search may compare the efficiency of downlink signal propagation paths from different sub-arrays of the second device to the first device. In some variations, the feedback signal may comprise a digital amplitude of the interrogation signal. One or more transducer configurations of the second device may select one or more of the sub-arrays corresponding to a maximum digital amplitude of the interrogation signal. In some variations, the feedback signal may comprise one or more of digital first device energy data and digital interrogation strength data. In some variations, the first device may comprise a power source comprising one or more of a rechargeable battery, capacitor, supercapacitor, and non-rechargeable battery. In some variations, digital first device energy data may comprise a power source parameter comprising one or more of a voltage, energy level, charging voltage, and charging current. Digital interrogation strength data may comprise one or more digital signals representing a strength (e.g., a voltage amplitude, power, and the like) of an interrogation signal received by the first device.

In some variations, data encoded by the first device in the feedback signal may comprise data may include, but is not limited to, the power or voltage (e.g., V_{OC}) received by the first device's one or more transducer elements due to an interrogation signal (e.g., after digitization of the power or voltage, or result of comparison of the power or voltage to a predetermined threshold), the first device's battery and/or capacitor voltage, battery charging current, DC voltage generated by the first device's power circuit after rectifying an interrogation signal (e.g., after DC combining, or power combining), combinations thereof, and the like. Encoded data may be used to determine the power or amplitude (e.g., voltage) of each driving signal comprising the selected transducer configuration and/or the duration for which power may need to be transferred to the first device.

For example, data corresponding to the power or voltage received by one or more transducers of the first device due to the interrogation signal, and/or the DC voltage generated by the first device's power circuit after rectifying an interrogation signal, may be directly useful for estimating the link gain (e.g., power received by the first device divided by the power transmitted by the second device). This may allow estimation of the amplitude or power of each driving signal comprising the selected transducer configuration, and/or the powering duration, in order to transfer a predetermined amount of power or energy to the first device. In some variations where a first device's power circuit comprises a re-chargeable battery, parameters related to the battery (e.g., battery and/or capacitor voltage, battery charging current, and the like) may be useful for estimating the DoD of the battery and/or the power or energy required for recharging the battery to a predetermined SoC.

FIG. 5 is a block diagram of a first device (510) comprising a transducer (520) configured to receive a downlink signal (540), such as an interrogation signal, from a second device (not shown). A power circuit of the first device (510) may comprise a power recovery circuit (552), a battery charging circuit (554), a battery (556), and a supply generation circuit (558). The power recovery circuit (552) may comprise circuits such as rectifiers, DC-DC converters, and the like. The battery charging circuit (554) may comprise one or more of a constant current (CC) charging circuit, a constant voltage (CV) charging circuit, combinations thereof, and the like. The battery (556) may be a rechargeable or secondary battery such as a rechargeable Lithium ion battery. Supply generation circuit (558) may be powered from the battery (556) and may generate one or more DC supply voltages and/or currents required by other circuit blocks. In some variations, the first device (510) may comprise a sensor (560) such as a pressure sensor. A processor of the first device (510) may comprise a sensing and processing circuit (532) and data communication circuit (534). The sensing and processing circuit (532) may be configured to sense a signal generated by the sensor (560), voltage received by the transducer (V_{P} or V_{OC}), battery voltage (V_{BAT}), battery charging current (I_{CHARGE}), combinations thereof, and the like. The sensing and processing circuit (532) may be configured to perform one or more of sensing, signal conditioning, digitizing, processing digital and/or analog signals, reading/writing data from/to memory (which may be included within the sensing and processing circuit, 532, or may be external to it), controlling one or more circuit blocks, providing/recovering data to/from the data communication circuit (534), combinations thereof, and the like. The data communication circuit (534) may be configured to transmit and/or receive data with the second device using the transducer (520).

In some variations, data encoded by the first device in the feedback signal may comprise data corresponding to the temperature at the first device. For example, the first device may comprise a temperature sensor configured to measure temperature data. The temperature data may be digitized. The measured temperature may be compared to a threshold, and the results encoded onto the feedback signal. In some variations, the second device may be configured to measure a temperature (e.g., skin temperature). Temperature data may be used by the second device to adjust the voltage and/or power of each driving signal comprising the selected transducer configuration, and/or the powering duration, in order to maintain the temperature within a safety limit.

In some variations, the feedback signal transmitted by a first device may comprise a broadband or an ultra-wideband (UWB) signal spanning a wide range of frequencies. In some variations, a processor of the second device may be configured to process the received feedback signal (e.g., may perform a fast Fourier transform or FFT) to determine one or more frequencies for transferring power to the first device. For example, one or more frequencies may be used for wireless powering of the first device may correspond to the received feedback signal comprising the highest power, sufficiently high power, or a high link gain (due to reciprocity). In some of these variations, the first device may comprise one or more ultrasonic transducer element(s) with a wide inductive band (frequency range where the transducer impedance may be inductive), a configurable impedance matching network (e.g., comprising one or more capacitors and switches), combinations thereof, and the like, for transmitting a broadband feedback signal. In some variations, the first device may be configured to transmit different ultrasonic pulses with different carrier frequencies.

In some variations, data corresponding to the power or voltage received by one or more transducers of the first device may be received from an interrogation signal. The second device may be configured to determine an orientation or rotation of the first device relative to the second device and/or monitor a change in the orientation or rotation of the first device over time based on the data. For example, a first device may comprise three ultrasonic transducers that may be positioned orthogonal to each other such that each ultrasonic transducer may preferably receive ultrasonic signals from a direction orthogonal to the preferred direction of the other ultrasonic transducers. For example, if an ultrasonic signal (e.g., an interrogation signal) from a second device is configured to arrive at a first device from a specific direction, and there is a change in the received power or voltage at the three ultrasonic transducers of the first device over time, it may indicate a relative rotation of the first device. In some variations, the data may be used by the second device to estimate a rotation of the first device during a cardiac or breathing cycle, and/or over the long term (e.g., over weeks, months or years). In some variations, the data may be processed to detect motion of one or more of the heart, heart wall, heart chamber (e.g., left ventricle or LV), a blood vessel, or any tissue structure that the first device may be implanted in or near. Additionally or alternatively, the data may be processed to diagnosis or monitor conditions such as heart failure. In some variations, the second device may alert a user and/or a doctor upon generation of a user prompt upon detection of the data.

In some variations, determining a selected transducer configuration based on a feedback signal may maximize the link efficiency and reliability of power transfer to the first device. In some variations, a first device may be configured to transmit a feedback signal (e.g., at least an analog feedback signal) using more than one transducer elements (e.g., all transducer elements of the first device). In some variations, the first device may be configured to transmit the feedback signal using more than one transducer elements one after the other (e.g., after a fixed delay), or simultaneously (e.g., at the same frequency or at different frequencies), or combinations thereof, and the like. A second device may be configured to receive the feedback signals and determine a selected transducer configuration based on the feedback signal with the highest overall power received by the second device. For example, a first device may be configured to transmit a feedback signal using each transducer element one by one (e.g., with the same transmit power for each transducer element) after receiving an interrogation signal from the second device. A processor of the second device may compute the overall or total power received by the second device (e.g., sum of the power received by all of its elements) from each feedback signal. The feedback signal with the highest total received power by the second device may correspond to the transducer element of the first device having the highest link gain with the second device.

In some variations, the feedback signal may comprise energy modalities such as RF or magnetic, as discussed herein. A processor of a second device may be configured to process the received RF feedback signals (e.g., analog feedback signal, digital data bits, and the like) in a similar manner as described herein. For example, the processor may be configured to perform triangulation to estimate a location of the first device, determine driving signals (e.g., phases, delays, power or amplitude for each transducer element) for a selected transducer configuration, combinations thereof, and the like. Upon the processing of an RF feedback signal, the second device may be configured to transfer power to the first device using any energy modality such as ultrasound, RF, magnetic, and the like.

In some variations, the second device may be configured to output one or more of features of the interrogation signal (e.g., the transducer element used to transmit the interrogation signal), features of the received feedback signal (e.g., amplitude of the received feedback signal at one or more transducer elements), data corresponding to the selected transducer configuration (e.g., which elements were selected for the selected transducer configuration), combinations thereof, and the like. In some variations, a user may be instructed by a user prompt to take an action (e.g., manually select one or more components of the selected transducer configuration, manually adjust or move the second device, and the like). For example, a user prompt may be generated to instruct manual repositioning of the second device when transducer elements of a selected transducer configuration lie on one side of the second device. Repositioning the second device may move the transducer elements near the center of the second device transducer array.

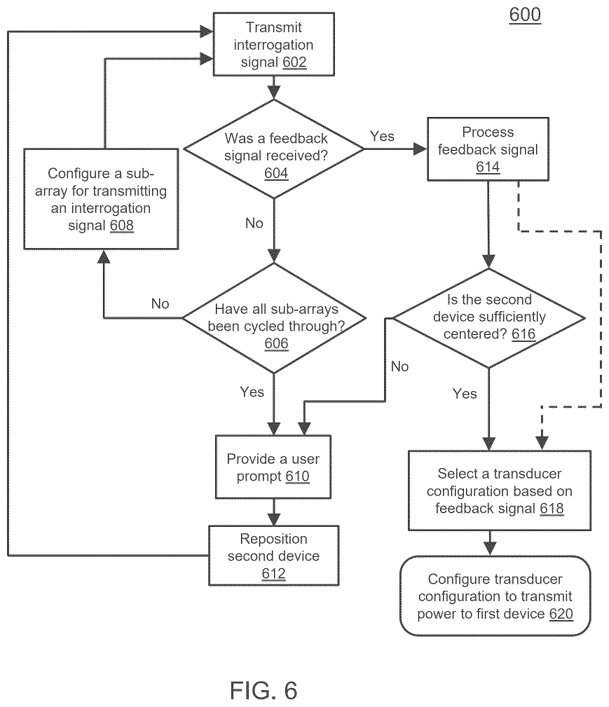

FIG. 6 shows an example flowchart of an illustrative variation of the methods described herein for interrogating a first device and transmitting wireless power. As shown, in some variations, an interrogation signal (IS) may be transmitted by a second device to a first device (602). The second device may check if a feedback signal (FS) is received from the first device or not (604). If no feedback signal is received (or the received feedback signal did not satisfy a predetermined condition), the second device may then configure a different transducer element (or a different sub-array) for transmitting an interrogation signal, cycling through a desired set of transducer elements one or more times (606 and 608), as described in detail herein. Other solutions described herein may also be used if no feedback signal is received by the second device.

In some variations, after a second device may have attempted different variations of transmitting the interrogation signal (e.g., after the second device may have cycled through all sub-array configured for transmitting interrogation signals, 606), a user prompt may be provided (610), and a user may be instructed to reposition the second device (612), as described in detail herein. These steps may then be repeated until the second device may successfully receive one or more feedback signals from the first device (or until the received feedback signal may satisfy a predetermined condition). If the second device successfully receives feedback signal (604), a processor of the second device may generate feedback signal data (FSD) by processing the feedback signal (614), as described in detail herein. As also discussed herein, in some variations, a second device may check, based on feedback signal data, if it is sufficiently centered relative to the first device or not (616). If it is determined that the second device is not sufficiently centered relative to the first device, a user prompt may be provided (610). A user may manually adjust or reposition the second device (612). The steps may be repeated until the second device may be sufficiently centered relative to the first device. In some variations, the process of centering the second device relative to the first device based on a user prompt and manual adjustment or repositioning may be skipped or bypassed, as shown by the dashed arrow in FIG. 6. The processor of the second device may then select a transducer configuration based on the feedback signal (618), and configure the transducer configuration to transmit power to the first device (620), as discussed in detail herein. As mentioned before, FIG. 6 only shows an example of a sequence of steps, and in some variations, these steps may be executed in a different order, or other combinations or sub-sets of methods described herein may be used to determine a sequence of steps, or a flowchart, for the interrogation of a first device and providing wireless power to it.

c. Interval-based Exchange of Wireless Signals

In some variations, an interval-based exchange of wireless signals may be used to efficiently power and/or communicate with a first device (e.g., an IMD) that follows a spatial path within the body. In some variations, wireless signals may be exchanged between a first device (e.g., an IMD) and a second device (e.g., a wireless device) during a plurality of intervals. The method may comprise the steps of transmitting an interrogation signal to a first device using a first sub-array of a second device, receiving a feedback signal from the first device using a second sub-array of the second device, selecting one or more transducer configurations of the second device based on the feedback signal, and exchanging one or more wireless signals with the first device using the one or more transducer configurations of the second device during a plurality of intervals, wherein the wireless signals comprise one or more of a power signal, data signal, interrogation signal, feedback signal, downlink signal and uplink signal. In some variations, the method may further comprise transmitting the feedback signal from the first device in response to one or more wireless signals (e.g., power signal, interrogation signal, data signal) received by the first device during one or more of the plurality of intervals. In some variations, the method may further comprise detecting one or more of a falling edge of one or more wireless signals and a code corresponding to one or more wireless signals (e.g., power signal, interrogation signal, data signal) received by the first device.

In some variations, the method may comprise determining to transmit one or more of a power signal, interrogation signal, data signal and a downlink signal to the first device in response to the received feedback signal. In some variations, the method may comprise determining to inhibit transmission of the wireless signal to the first device in response to the received feedback signal. For example, in some variations, if the strength of the received feedback signal is measured to be greater than a predetermined threshold, it may be determined that the link efficiency between the second device and the first device is favorable, and the second device may decide to transmit a power signal to the first device. In some variations, if the strength of the received feedback is measured to be below a predetermined threshold, the second device may decide to not transmit any wireless signal to the first device and/or transmit a wireless signal (e.g., an interrogation signal) after a wait time.

In some variations, the transducer configuration corresponding to a subsequent interval may be selected based on one or more previously received feedback signals during one or more previous intervals. In some variations, a duration of at least one interval of the plurality of intervals may be determined by the first device. In some variations, a duration of at least one interval of the plurality of intervals may be determined by the second device. In some variations, the first device may be configured to periodically transmit the feedback signals during one or more of the intervals.

As an example of example of interval-based exchange of wireless signals, a variation of interval-based powering is described herein, where a power signal may be transmitted by the second device to the first device during a plurality of power intervals. However, it is understood that such a method may generally apply to an exchange of any type of wireless signals between a first device and a second device during a plurality of intervals.

In some variations, interval-based powering may efficiently power a first device that follows a spatial path within the body. For example, a miniature (e.g., millimeter-sized) IMD implanted in the heart may move/rotate due heart wall motion and/or breathing. A transducer configuration may be selected for transmitting wireless power to the first device at a predetermined time period configured for efficient power transfer.

In some variations, a method of interval-based powering may comprise transferring power to a first device in different time intervals (referred to as a power interval). A transducer configuration may be determined independently, as described before, for each power interval. An example is presented herein for powering a moving IMD implanted in or near the heart or a heart chamber.

In some variations, a second device may be configured to transmit an interrogation signal to a first device, receive a first feedback signal from the first device, process the first feedback signal to generate first feedback signal data, and determine a first selected transducer configuration at least based on the first feedback signal data. The second device may configure the first selected transducer configuration to transfer power to the first device during a first power interval. At the end of the first power interval, the first device may be configured to transmit a second feedback signal to the second device. The second device may process the second feedback signal to generate second feedback signal data, and determine a second selected transducer configuration based on at least the second feedback signal data. The process of transmitting a feedback signal after a power interval to determine the selected transducer configuration for the next power interval may be repeated until one or more predetermined conditions are met (e.g., sufficient amount of power or energy is transferred to the first device). Different variations of the feedback signal, feedback signal data, and selected transducer configuration, as described herein are applicable herein.

In some variations, the second feedback signal transmitted by a first device at the end of a first power interval may encode data corresponding to the power signal received by the first device during the first power interval. The second feedback signal may encode data in a manner analogous to encoding data for an interrogation signal as described herein. For example, in some variations, a second feedback signal may include, but is not limited to, a signal acknowledging the receipt of power during a first power interval, an analog feedback signal, data corresponding to the power or voltage received by one or more transducer elements of the first device during and/or at the end of the first power interval (e.g., after digitization of the power or voltage, or result of comparison of the power or voltage to a predetermined threshold), the first device's battery voltage during and/or at the end of the first power interval, battery charging current during and/or at the end of the first power interval, a DC voltage generated by the first device's power circuit during and/or at the end of the first power interval, combinations thereof, and the like.

In some variations, the duration of a single power interval may be predetermined or may be determined in real time during the wireless powering of the first device. In some variations, the duration of a power interval may be determined by the first device and/or by the second device. For example, a predetermined duration may be based on prior knowledge of the motion or speed of the first device, and effects or factors causing such motion. For example, a miniature first device (e.g., IMD) attached to a heart wall may move periodically with a time period of about 1 second for a heart rate of about 60 beats/minute (ignoring the effect of breathing for simplicity). Assuming that the first device traverses a total path length (round trip) of about 10 cm in one period, as an example, and assuming that the speed of the first device may be constant over time, it may be estimated that in a duration of about 10 ms, the first device may move by about 1 mm. If the ultrasonic transducer of the first device comprises a width of about 1 mm and if the powering beam comprises a half-power beam diameter of about 4 mm, then the duration of the power interval may be set to about 10 ms, after which a new transducer configuration may be determined in order to reliably power the first device in its new position. In some variations, the motion, path, or trajectory of a first device may be mapped using any technique such as imaging or triangulation based on an analog feedback signal, and the like. The spatial path may be used to determine a duration of the power interval. In some variations, the duration of the power interval may be between about 1 ms to about 100 ms. In some variations, a set of power intervals for powering a first device may either have the same duration or may have different durations. In some variations, a set of durations of the power interval may be determined based on knowledge of the motion of a first device. For example, if it is known or determined that a first device may not move significantly during a large time window (e.g., about 300 ms) within a cardiac cycle (e.g., a diastole), then a second device may estimate such a time window of the cardiac cycle (e.g., by measuring heart rate or ECG), and may use a longer duration of the power interval (e.g., about 300 ms) during that time window.

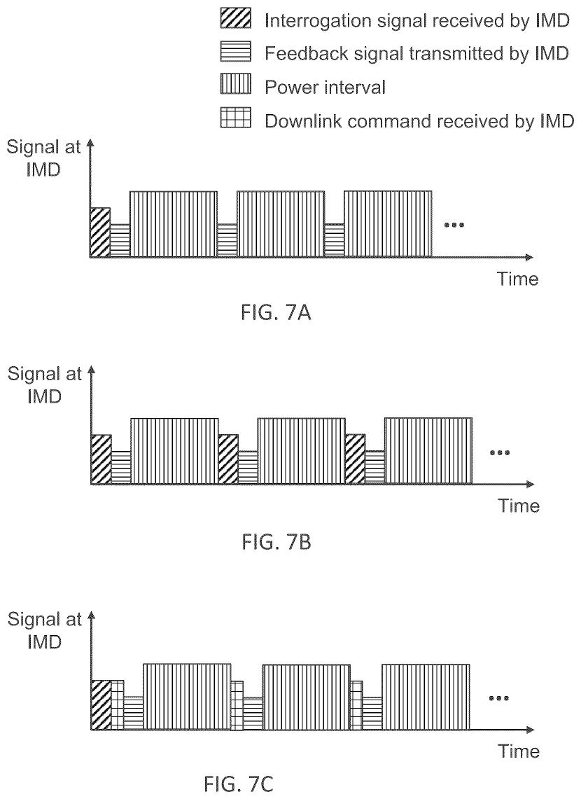

In some variations, a first device may be configured to transmit a feedback signal at the end of a power interval (e.g., in addition to being configured to transmit a feedback signal after receiving an interrogation signal). This variation is illustrated in FIG. 7A, where different signals at a first device (e.g., an IMD) are conceptually represented on a timing diagram. It should be noted that there may be a finite non-zero time delay between different signals, and/or the signal amplitudes and/or durations may be different than what is conceptually depicted in the figures. In some variations, a first device may be configured to detect a falling edge of the voltage envelope received by one or more transducer elements of the first device or a reduction in its received power or voltage, in general, below a predetermined threshold. The falling edge or reduction in the received power or voltage of the first device may be due to the second device ending the power interval or due to the position, or orientation of the first device changing significantly while the second device is still transmitting power in a power interval, or both. In some variations, the first device may be configured to detect a code of one or more wireless signals (e.g., interrogation signal, power signal, data signal) transmitted by the second device.

In some variations, as shown in FIG. 7B, the second device may transmit an interrogation signal after a power interval. The first device may respond to the interrogation signal by transmitting a feedback signal which may be received and processed by the second device to determine the selected transducer configuration for the next power interval. In some variations, the second device may be configured to transmit a specific downlink code or command to the first device during and/or at the end of a power interval. The command may specifically trigger the first device to transmit a feedback signal, and the first device may transmit a feedback signal upon detecting the code or command, as conceptually illustrated in FIG. 7C.

In some variations, the second device may stop transmitting wireless power to the first device based on feedback signal data. For example, a feedback signal from a first device may encode the voltage of a first device battery, and a processor of the second device may decode the encoded voltage. The processor may determine an SoC of the first device battery and stop wireless power transfer to the first device if a desired or maximum SoC has been reached. In some variations, the first device may transmit a command to the second device to stop transmitting wireless power when a certain condition is met (e.g., when the first device battery has reached a desired or maximum SoC).

In some variations, at any or all times during the execution of one or more methods described herein, a second device may generate a user prompt (e.g., user feedback). A user prompt may include, but is not limited to, the SoC of the first device, the SoC of the second device, whether charging of the first device is complete, whether charging of the first device is interrupted due to any reason, combinations thereof, and the like. Interruptions in charging or wireless powering of a first device may be due to reasons including, but not limited to, a user moving or removing a second device from a patient's body, second device falling off or getting displaced or disconnected, second device's battery running low, combinations thereof, and the like. A user may take an action in response to the user prompt such as recharging the second device's battery if the second device's battery is running low, re-positioning the second device if the second device is displaced, turning off and removing the second device if IMD's charging is complete, combinations thereof, and the like.

In some variations, a second device may be configured to save data corresponding to a motion of the first device, and/or a selected transducer configuration corresponding to a location of the first device, to the memory of the second device, and use the data for one or more subsequent power intervals. The data may comprise one or more of a position of the first device (e.g., one or more spatial coordinates and/or orientations or rotation of a first device, position of a first device as a function of time), parameters of a selected transducer configuration (e.g., selection of transducer elements, driving signals for the transducer elements, and the like), a temporal parameter related to the motion of the first device (e.g., heart rate, breathing rate, and the like), speed and/or acceleration of the first device relative to the second device, combinations thereof, and the like. For example, in some variations, the spatial coordinates of a first device, or the selected transducer configurations, as a function of time over one or more cardiac/breathing cycles may be saved in the memory of the second device. The spatial coordinates may be used by the second device for reliably powering the first device in one or more next cardiac/breathing cycles. In some of these variations, the second device may not have to determine a new selected transducer configuration for each power interval in real time (which may help with saving computation time and energy), and may rely on previously determined selected transducer configurations to reliably power the first device. In some variations, selected transducer configurations may generate a focal spot size at the first device that may be larger than the dimensions of one or more transducers of the first device (e.g., focal spot diameter may be 4 times the diameter of a first device's transducer). This may reduce sensitivity of the first device's received power to one or more of small deviations from its trajectory for which the selected transducer configurations may have been saved in the second device's memory, other link aberrations, combinations thereof, and the like.

i. Intermittent Exchange of Wireless Signals

In some variations, the first device may temporarily move or rotate in a manner such that the link gain between the second device (e.g., external wireless device, wireless device) and the first device may deteriorate for a portion of a spatial path (e.g., trajectory) of the first device. For example, during a heartbeat (or due to breathing), a first device attached to a heart wall may move temporarily to a position behind a lung or a rib such that an ultrasonic beam from a second device to the first device may be partially or fully obstructed or attenuated. Exchanging wireless signals (e.g., power, data or other signals) with a first device during such a time may be inefficient. For example, it may require a large transmit power from the second device due to low link gain, and may result in unnecessary heating of tissue. Solutions provided herein may be useful to overcome such a challenge.

In some variations, a method of intermittent exchange of wireless signals may be used as described herein. In some variations, a second device may be configured to inhibit transfer of a wireless signal (e.g., power, data, interrogation signal) to a first device in response to a feedback signal during a method of interval-based exchange of wireless signals. An example of intermittent powering is described herein, where the second device may be configured to inhibit powering of the first device during one or more intervals. However, it is understood that such a method may generally apply to exchange of any type of wireless signals between a first device and a second device during a plurality of intervals.

In some variations, a received power and/or voltage of an analog feedback signal that is significantly lower compared to a previously received feedback signal (e.g., 6 dB lower power compared to a previously received feedback signal's power) may correspond to movement and/or rotation of the first device to an unfavorable position/configuration with a significantly low link gain. In some of these variations, powering the first device at a different time when the link gain is more favorable may be preferable to transmitting a large power from the second device to compensate for the low link gain. In some variations, the second device may be configured to make the determination of not transferring wireless power based on processing one or more of the analog feedback signal, feedback signal comprising digital data bits encoding the power or voltage received by the first device's one or more transducer elements during and/or at the end of the first power interval, a DC voltage generated by the first device's power circuit during and/or at the end of the first power interval, combinations thereof, and the like.

In some variations, one or more components of feedback signal data (e.g., power received by a first device's transducer element) may be compared to the corresponding components of the feedback signal data generated previously during interval-based powering, or one or more components of feedback signal data may be compared to one or more predetermined (e.g., absolute) thresholds to determine whether to transfer wireless power in the next power interval or not. This method may be referred to as intermittent powering. Intermittent powering may efficiently use energy, extend second device battery life, and avoid unnecessary heating of tissue.

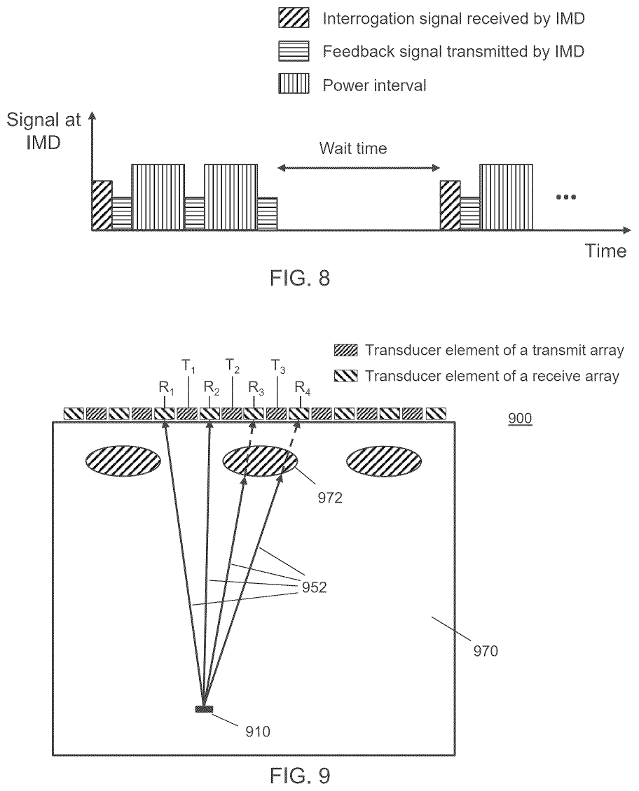

In some variations, a second device may determine to not transfer power to a first device during a particular power interval, and then determine how and when the wireless powering of the first device may be resumed. In some variations, in response to the determination to not transfer power, the second device may wait for a predetermined time delay before transmitting one or more interrogation signals to the first device. Depending on receipt of this interrogation signal by the first device and receipt of the corresponding feedback signal by the second device, the second device may resume power transfer (e.g., resume interval-based powering) to the first device, as described before. As depicted in FIG. 8, the second device may be configured to transmit the interrogation signal after a time delay (e.g., a wait time) of between about 1 ms and about 500 ms. For example, in some variations, a wait time may be about 100 ms that may be sufficient for the first device to move/rotate back to a position/orientation where its link gain with the second device may be favorable or sufficiently high. In some variations, the second device may continue to send interrogation signals to the first device (e.g., periodically every 10 ms or every 100 ms, and the like) until it receives a feedback signal from the first device that indicates a favorable link gain.

In some variations, a second device may determine to not transfer power to a first device. The first device may then be configured to transmit one or more feedback signals to the second device after waiting for a predetermined time delay (e.g., periodically every 10 ms or 100 ms, and the like) without being explicitly interrogated by the second device. In some of these variations, the second device may wait to receive a feedback signal from the first device. In some of these variations, the first device may use stored energy (e.g., from a battery) to transmit the one or more feedback signals. In some variations, the first device may be configured to transmit one or more feedback signals until one or more of the following conditions are met including, but not limited to, the first device receiving power from the second device in a next power interval, the second device sending a command to the first device to stop transmitting feedback signals, the first device transmitting a predetermined number of feedback signals or for a predetermined time duration, combinations thereof, and the like.

d. Transducer Configuration Based on Reflected Signals

In some variations, the feedback signal may comprise an active uplink signal transmitted by a first device (e.g., upon detecting an interrogation signal) such as a first device. An active uplink signal may provide several advantages such as a large received signal or signal-to-noise ratio at the second device, flexibility in choosing the uplink signal's frequency, and duration relative to the interrogation signal. However, in some applications, detecting an interrogation signal may require a wake-up receiver capable of detecting low interrogation signal levels. In some variations, a wake-up receiver may consume large amounts of energy. Therefore, a first device comprising a wake-up receiver may require a larger battery.

In some variations, ultrasound imaging may be used to locate a first device. For example, ultrasound imaging may include beamforming to sweep or scan a focused beam over an area or volume of tissue. However, ultrasound imaging may require a relatively long time to scan a large tissue region (e.g., several centimeters in all three dimensions) using a small focal spot size (e.g., millimeter diameter), complex processing capability, and/or high power consumption of the second device.

In some variations, a sub-array of a second device may be configured to transmit an ultrasonic interrogation signal into tissue. The corresponding feedback signal may comprise one or more ultrasonic reflection signals from one or more first devices. The received reflection signals may be processed by a processor of the second device to generate feedback signal data and to identify which portion(s) or feature(s) of the reflection signal may correspond to the first devices. This may be used to determine a transducer configuration using time reversal techniques or any other search/beamforming techniques. The transducer configuration may be used to focus at the location of the first device for transmitting power, data, and/or other signals to the first device, and/or for receiving data and/or other signals from the first device.

In some variations, a sub-array may be configured to transmit an interrogation signal may comprise one or more ultrasonic transducer elements of the second device. The second device may be configured to generate a spatially broad or an unfocused beam in tissue (e.g., a plane wave, an approximate plane wave) referred to as low-gain transmit. For example, a half-power beam diameter of the interrogation signal near the first device may be more than about two times the maximum lateral dimension of the transducer of the first device that receives the interrogation signal. An unfocused beam may allow a quick scan of a large tissue volume for locating a first device. In some variations, transducer elements or sub-arrays of the second device may be cycled through (e.g., configured as sub-array one-by-one) to scan tissue until a transducer configuration is determined according to predetermined criteria. In some variations, one or more transducer elements may be configured to transmit an interrogation signal with a relatively focused beam (e.g., high-gain transmit) in order to reduce transmit power requirements. In some variations, interrogation may be performed using a full beamforming scan (e.g., adjusting the phase of one or more transducer elements of the sub-array to scan the beam at different angles).

In some variations, an ultrasonic interrogation signal comprise a short pulse width (e.g., a few microseconds to tens of microseconds) to enable sufficient resolution (e.g., on the order of millimeters to centimeters) to distinguish between different structures such as the first device, ribs, lungs, and the like, based on reflections of the interrogation signal. A short pulse width corresponds to a large bandwidth, and may require wideband transducer elements and result in a large minimum detectable signal (MDS) requirement for the reflection signals received by the second device (due to integration of noise over a large bandwidth). Thus, in some variations, an ultrasonic interrogation signal with a longer pulse width (e.g., on the order of hundreds of microseconds, milliseconds, or more) may be used to relax the bandwidth requirement (e.g., allowing the use of transducer elements with a lower bandwidth). In some variations, an end or falling transient of the pulses of the reflection signals may be processed to determine a transducer configuration.

In some variations, a carrier frequency of an interrogation signal may be the same as the power or data transfer frequency, which may enable accurate modeling of the link and determination of a transducer configuration for efficient power or data transfer. In some variations, a different carrier frequency may be used for interrogation. For example, in some variations, a higher carrier frequency may be used to transmit a shorter pulse for achieving a higher resolution in identifying a first device. In some variations, a lower carrier frequency may be used to reduce propagation losses of the interrogation signal through tissue, thereby increasing the strength of the reflection signal from the first device, and/or reducing the transmit power requirement. The transmit power required for this method may be estimated based on the MDS at one or more transducer elements of the second device, tissue-based losses, radar cross-section of the first device, and the radar range equation. In some variations, a carrier frequency for the interrogation signal may be selected to increase the strength of a reflection signal from the first device (e.g., from the first device's transducer), and/or may result in a uniquely identifiable different frequency component in the reflection signal from the first device due to non-linear backscattering.

In some variations, an interrogation signal transmitted by a sub-array of the second device into tissue may generate one or more feedback signals comprising one or more reflection signals. The reflection signals may comprise reflections off of one or more first devices and/or one or more tissue structures such as ribs, lungs, boundaries between two types of tissue, combinations thereof, and the like. In some variations, a time window or time delay based on the propagation speed of ultrasound in tissue may be used to record reflection signals from a desired tissue depth, or a set or range of tissue depths. In some variations, three or more transducer elements of the second device may be used for triangulation of the first device's location based on processing the feedback signal.

In some variations, the second device may comprise one array of transducer elements, wherein the transducer element(s) comprising the TTC and the RTC may be selected from this array, for a predetermined iteration of the method described here. In some variations, a first subset of the array may be configured only for transmitting, and one or more transducer element(s) comprising the TTC may be selected from the first subset. In some variations, a second subset of the array may be configured only for receiving, and one or more transducer element(s) comprising the RTC may be selected from the second subset. In some variations, the second device may comprise two arrays. A predetermined array may be configured to either transmit or receive signals. In some of these variations, transducer elements comprising the TTC may be separate (e.g., distinct) from transducer elements comprising the RTC.

In some variations, the feedback signal comprising reflection signals may be processed by a processor of the second device to generate feedback signal data in order to estimate a geometry and/or size of a source of reflection. The feedback signal may further be used to identify which portion(s) of the reflection signals may correspond to the first device, as opposed to tissue structures such as ribs, lungs, and the like. The processing may be performed in one or more ways. For example, if an interrogation signal is in the form of an ultrasonic pulse, a reflection signal received by one or more transducer elements of the second device may comprise one or more ultrasonic pulses. Waveform features of a received reflection signal such as number of pulses, amplitude, phase, delay and/or frequency of one or more pulses, and/or the variation of waveform features across different transducer elements of the second device may depend on the position, geometry, size and/or properties of the source of reflection.

In some variations, the amplitude, phase and/or delay features of a reflection signal and/or of one or more pulses in the reflection signal may be compared across one or more transducer elements of the second device in order to distinguish between different sources of reflection. Reflections from a first device may originate from a single small spot in tissue whereas reflections from ribs may originate from multiple spots (or a periodic grid) and those from lungs may originate from a large surface area. In some variations, knowledge of an approximate tissue depth or a range of tissue depths of the first device may be used to identify which portion of the reflection signal corresponds to the first device. For example, since ribs may be located at shallow tissue depths (e.g., less than about 2 cm) and a first device may be located at a deeper tissue depth (e.g., greater than about 2 cm), reflections from ribs may arrive at the second device at an earlier time compared to reflections form the first device. In some variations, an n^{th} reflection event (where n is an integer), or an n^{th} pulse in a received reflection signal comprising a plurality of pulses may be known to be from a first device. For example, a fourth reflection or a fourth pulse in a received reflection signal may be known to correspond to a first device, whereas the first, second, and third reflections may be known to correspond to reflections from the skin, ribs and/or other structures. In some variations, the processor of the second device may be configured to process the reflection signal to detect an additional frequency (apart from the frequency of the interrogation signal) to identify which portion of the reflection signal or which pulse of the reflection signal corresponds to a reflection from the first device. For example, an interrogation signal incident on a first device may undergo non-linear backscattering resulting in a different frequency component in its reflection. Non-linear backscattering may be a useful signature of the received reflection signal to identify the first device.

Identification of the portion(s) of a received reflection signal that correspond to a first device may be used to determine a transducer configuration for efficiently exchanging wireless signals with (e.g., transmitting to, receiving from) the first device. In some variations, predetermined transducer elements of the second device which receive a substantially low reflection signal amplitude from the first device compared to other transducer elements may not be used as a portion of the transducer configuration to transmit power to the first device. For example, the path from the transducer elements to the first device may be blocked by a rib. In some variations, relative time delays and/or amplitudes of the reflection signal of the first device received at one or more transducer elements of the second device may be reversed while transmitting power. This may focus the transmit beam at the location of the first device. In some variations, relative time delays of the first device's reflection signal at three or more transducer elements of the second device may be used to estimate a relative position or a range or positions of the first device in tissue (e.g., using triangulation). In some variations, after estimating an approximate position of the first device in tissue, a transducer configuration may be determined to comprise a sub-array powering/data snapshot. For example, a sub-array powering snapshot may refer to a selected set of transducer elements of the second device (e.g., a sub-array), along with their driving signals (e.g., amplitude, frequency, phase), configured to selectively focus power at the location of the first device. In some variations, after estimating an approximate position of the first device in tissue, phased array beamforming using a subset of transducer elements of the second device or all transducer elements of the second device may be used to focus at the location of the first device for efficiently exchanging wireless signals.

In some variations, a downlink-based search for the transducer configuration may be performed with the feedback signal comprising one or more ultrasonic reflection signals from one or more first devices or tissue. For example, the sub-array may comprise a sub-array configured to transmit an interrogation signal into tissue, and the second device may be configured to receive and process reflection signals from tissue and/or the first device in order to identify an approximate location of the first device. The second device may perform an A-scan (amplitude scan) or a B-scan (brightness mode scan), and the like, to identify a reflection from a first device and to estimate its location in tissue. This process may be repeated for a predetermined number of different sub-arrays in order to search for the transducer configuration which has the highest ultrasonic link gain with the first device. For example, a larger amplitude or echo in the A-scan corresponding to a predetermined sub-array may indicate that that sub-array has a higher ultrasonic link gain with the first device and may be designated as the selected transducer configuration.

In some variations, the steps described herein may be repeated periodically to track a first device that moves relative to a second device due to breathing, beating of the heart, and the like. For example, in some variations, the method may be applied to interval-based power and data transfer. A transducer configuration may be determined or updated periodically for exchanging wireless signals with a moving first device during a time interval. In some variations, the interrogation signal may be changed over subsequent intervals during interval-based power and data transfer. For example, one or more different transducer elements may be configured as a sub-array for transmitting interrogation signals for different intervals. In some variations, an interrogation signal may not be transmitted for all intervals and instead, reflections of the power signal transmitted by a transducer configuration of a previous interval may be processed to determine a transducer configuration for the next interval.

In some variations, the received reflection signals may be processed to identify which portions of the reflection signal correspond to a plurality of first devices. One or more transducer configurations may be determined to efficiently exchange wireless signals with the plurality of first devices, simultaneously or at different times.

An interrogation signal in the form of a short pulse spanning a wide frequency band may undergo dispersion in tissue, causing the reflected feedback signal to have one or more pulses that are smoothed (i.e., having gradual rising/falling transients). This may be due to the frequency dependence of the sub-array gain (e.g., limited bandwidth), first device radar cross-section, and/or link dispersion. While short pulses may offer a high axial resolution, dispersion may make it challenging to identify portion(s) of the reflection signal that correspond to a first device, estimate a location of the first device in tissue, and/or determine a transducer configuration using time reversal or other search/beamforming techniques.

In some variations, feedback signal data may comprise relative phase, amplitude and/or time delay, i.e., a difference in these waveform features of the received reflected signals, across the transducer elements of the second device. This may be useful where feedback signals received by different transducer elements may have undergone a similar level of dispersion. For example, relative time delays between smoothed pulses (corresponding to the reflection from a first device), received by transducer elements of the second device, may be accurately determined by using a rising/falling edge detector circuit (e.g., comprising an envelope detector and a comparator or Schmitt trigger), and computing the time difference between the output pulses of the edge detector circuit. In some variations, the relative time delays may be used to determine a transducer configuration based on time reversal for efficiently powering the first device. In some variations, to address dispersion, an interrogation signal with a long pulse width (e.g., hundreds of microseconds, milliseconds, and the like), or low bandwidth may be used. However, this may result in reduced axial resolution, thus making it challenging in some applications to distinguish reflection signals coming from the first device from those of tissue structures such as ribs, lungs, and the like.

In some variations, the second device may be configured to receive feedback signals in one or more time windows, or after a predetermined time delay, to capture the last few cycles, or the falling edge, of the long received pulse corresponding to a reflection from a first device. For example, for first devices implanted in/near the heart, configuring the second device to receive feedback signals after a predetermined time delay may allow ignoring reflections due to shallow tissue structures such as ribs or skin, and only processing reflection signals from the first device and any deeper tissue structures to generate the feedback signal data. In some variations, the second device may be configured to receive or record all reflections, and analog and/or digital post-processing may be used to identify the last few cycles, or the falling edge, of the long pulse corresponding to the reflection from a first device. The relative time delays of the last few cycles, or the falling edge, across different transducer elements may be used to determine a transducer configuration for efficiently exchanging wireless power/data with the first device.

In some variations, the interrogation signal may comprise a range of frequencies (e.g., a chirp signal). In some variations of an ultrasonic interrogation signal, the range of frequencies may include a frequency (e.g., a range centered around the frequency) at which the size of the first device and/or a component of the first device (e.g., one or more ultrasound transducers of the first device) may be on the order of the wavelength (or a multiple of the wavelength). The radar cross-section (RCS) of a first device and/or a component of the first device (e.g., an ultrasound transducer) may change drastically around the frequency due to resonance effects (e.g., the RCS may have a local maximum, a local minimum, may oscillate with frequency, and the like, in this frequency range). This phenomenon may result in a unique signature of a mm-sized first device in the received feedback signal, which may be useful to distinguish it from larger cm-sized tissue structures such as ribs, lungs, and the like.

e. Transducer Configuration Based on Backscatter Signals

In some variations, a feedback signal from a first device (e.g., an IMD) may comprise a backscatter signal such as an ultrasonic backscatter signal. In some variations, a first device may be in a first mode such as a sensing mode before a second device (e.g., an external wireless device) sends an interrogation signal to it. In some variations, a second device may send a first interrogation signal to the first device, upon receiving which the first device may configure itself into a second mode that may be useful for determining a transducer configuration for efficiently exchanging wireless power/data with the first device. In a variation of the method described here, such a second mode may be a backscatter mode. The first device may be configured to backscatter an incoming interrogation signal as described here in detail. In some variations, stored energy on the first device (e.g., in a battery or a capacitor) may be useful for configuring the first device for backscattering (e.g., for modulating a load circuit described here). The second device may send a second interrogation signal that may be received by the first device in the second mode and use the corresponding feedback signal from the first device (e.g., backscatter signal from the first device corresponding to the second interrogation signal) to determine the transducer configuration. In some variations, a second mode may not be needed, and the first device may always be configured to backscatter an incoming interrogation signal.

In some variations, the first device may comprise one or more ultrasonic transducers coupled to one or more circuits that may be used to influence or modulate the ultrasonic backscatter signal from the first device in response to one or more interrogation signals sent by the second device. In general, influencing or modulating the backscatter signal from the first device may result in a unique signature of the first device in the feedback signals, and/or may improve the signal-to-noise ratio (SNR) of the feedback signals coming from the first device, which may be useful for reliably locating the first device.

In some variations, the circuit coupled to the ultrasonic transducer may comprise one or more of a load circuit, a rectifier or power recovery circuit, combinations thereof, and the like. In some variations, the circuit may be configured to modulate the amplitude, phase and/or one or more frequency components of the backscatter signal (e.g., adding a new frequency component to the backscatter signal relative to the interrogation signal).

In some variations, the load circuit coupled to the ultrasonic transducer of the first device may comprise one or more of a short, an open, one or more switches, one or more resistors, one or more reactive impedances (e.g., capacitors), one or more modulated impedances, combinations thereof, and the like. For example, shorting or connecting a small impedance across the terminals of an ultrasonic transducer may increase an amplitude of the backscatter signal, allowing the second device's processor to reliably detect the first device. In some variations, instead of a short circuit, a predetermined impedance may be coupled across the ultrasonic transducer. This may be useful to measure the strength of the interrogation signal received by the first device (e.g., voltage or power received by the ultrasonic transducer), and/or to recover some power from the interrogation signal while still increasing an amplitude of the backscatter signal from the first device.

In some variations, the load circuit may comprise a modulated impedance that may comprise any impedance that may be modulated or which may change as a function of time. A change in the modulated impedance may result in a modulation of the backscatter signal (which may be called a modulated backscatter signal) that may be detected by the processor of the second device, and used to locate the first device. The modulation of the backscatter signal may comprise a change in one or more of the amplitude, frequency, and/or phase relative to the incoming interrogation signal, and/or a variation in one or more of the amplitude, frequency and/or phase as a function of time. For example, in some variations, a processor of the first device may switch the impedance seen by the ultrasonic transducer between two or more values (e.g., between an open circuit and a short circuit, and the like), periodically or at one or more modulation frequencies. This may result in the modulated backscatter signal having a frequency component or a tone equal to a modulation frequency and/or its harmonic, which may be detected by the processor of the second device to locate the first device and/or to determine a transducer configuration to efficiently power the first device.

In some variations, the modulation frequency may be about equal to a frequency used for powering or transmitting downlink data to the first device. This may be useful for time reversal since it may allow measuring time delays or phases of the received feedback signal at the powering frequency. For example, if the desired powering frequency in an ultrasonic link is about 1 MHz, then an interrogation signal may be transmitted at a higher frequency (e.g., about 2 MHz), and the backscatter signal may be modulated with a modulation frequency of about 1 MHz. The processor of a second device may measure time delays or phases of the component of the feedback signal at about 1 MHz, and use those time delays or phases for time reversal to efficiently power the first device at about 1 MHz.

In some variations, the modulation frequency may be different from the frequency used for transmitting power or data to the first device. For example, in some variations, the modulation frequency may be a low frequency (e.g., about 100 kHz) that experiences low propagation losses through tissue, thereby allowing reliable detection of the modulated backscatter signal by the second device. In some variations, the modulation frequency may be a multiple of the powering frequency or vice versa. In some variations, the processor of the second device may process time delays or phases of the received feedback signal at the modulation frequency. The processor may further determine the required time delays or phases for transmitting power to the first device at the powering frequency using time reversal.

In some variations, the load circuit may be modulated to encode digital data, which may allow the second device to uniquely identify and/or locate a first device. The digital data may comprise one or more of an ID code, command, code to acknowledge receipt of the interrogation signal by the first device, code representing digitized energy state of the first device (e.g., its battery voltage), combinations thereof, and the like.

In some variations, the load circuit may be modulated to create one or more nulls or notches in the backscatter signal from the first device. A null or a notch may comprise a portion of the feedback signal received by the second device where the feedback signal amplitude is low or close to zero. In some variations, a null or a notch in the backscatter signal may be generated by switching the load circuit impedance between an open circuit and a short circuit. The processor of the second device may be configured to detect a null or a notch in the received feedback signal to uniquely identify and/or locate the first device. For example, the second device may measure relative time delays of the notch on different transducer elements, and process the time delays in order to determine a transducer configuration using time reversal or triangulation, and the like.

In some variations, a rectifier circuit or a power recovery circuit coupled to the ultrasonic transducer may result in a backscatter signal with a harmonic (e.g., a third harmonic) of the interrogation signal frequency due to the non-linear impedance (e.g., diodes) of the rectifier or power recovery circuit. The additional frequency component may only exist in the backscatter signal coming from the first device, as opposed to other reflections of the interrogation signal, may be used by the second device's processor to locate the first device.

In some variations, a downlink-based search for the transducer configuration may be performed with the feedback signal comprising one or more backscatter signals. For example, a sub-array may be configured to transmit an interrogation signal into tissue and the second device may be configured to receive and process backscatter signals from the first device. The process may be repeated for different sub-arrays in order to search for the optimal sub-array which has the highest ultrasonic link gain with the first device. For example, for a predetermined sub-array, a modulated backscatter signal may be detected using an envelope detection of the received feedback signal. The detected signal may denote that the sub-array has a sufficient ultrasonic link gain with the first device. Furthermore, the sub-array may be selected as the transducer configuration for efficiently exchanging power/data with the first device.

f. Second Device Array Configuration

Systems and methods described herein are configured to exchange wireless signals between a first device (e.g., an IMD) and a second device (e.g., a wireless device). In applications such as ultrasound imaging, it may be typical to use a single transducer array comprising one or more transducer elements that are configured to both transmit and receive signals. However, a second device may comprise two or more separate arrays with each array comprising one or more transducer elements. One or more arrays may be configured to transmit signals (transmit array) and one or more arrays may be configured to receive signals (receive array). For example, in some variations, a second device may comprise one or more transmit arrays, one or more receive arrays, and one or more arrays which may be configured to both transmit and receive signals. The array configurations described herein may preclude or reduce the use of transmit and receive switches typically used for configuring transducer elements for both transmitting and receiving signals so as to reduce design complexity and/or power dissipation of the second device. The array configurations may further enable design flexibility by decoupling design constraints on transducer elements and electronics for achieving transmit and receive functions. In some variations, transducer elements of the transmit and receive arrays may be of the same or different types, shapes, materials, dimensions, and the like. For systems comprising separate transmit and receive arrays, determining a transducer configuration for transmitting downlink signals (power, data, and the like) to a first device may be difficult. For example, feedback signals received on the receive array comprise transducer elements not configured for transmitting wireless signals to the first device.

In general, geometric relationships between element(s) of the transmit array and element(s) of the receive array may be used by the processor of the second device to determine drive signals for the transmit array element(s) based on received feedback signals on the receive array element(s). In some variations, the transmit and receive arrays may be partially or fully interleaved or interspersed with each other. For example, a transducer element of a transmit array may be positioned after every one or more transducer elements of a receive array or vice versa, in a periodic manner. In some variations, every alternate transducer element may belong to one type of array (transmit or receive). In some variations, the transmit and receive arrays may not be interleaved with each other (i.e., may be spatially non-overlapping). In some variations, a processor of the second device may process feedback signals received by one or more transducer elements of the receive array to generate feedback signal data, and may use interpolation and/or extrapolation of the feedback signal data, based on the relative spatial positions or geometry of transmit and receive array elements, to determine a transducer configuration for efficiently transmitting power/data to the first device.

FIG. 9 depicts a variation where each alternate transducer element may belong to either a transmit array or a receive array. Feedback signals (952) generated from a first device (910) may comprise one or more of reflection signals, active uplink signals transmitted by the first device, and the like. In some variations, the amplitude, phase and/or time delay of the feedback signals (952) received by the transducer elements of the receive array (e.g., R₁-R₄) may be processed to estimate which transducer elements of the transmit array (e.g., T₁-T₃) may be selected to comprise the transducer configuration and the driving signals thereof. For example, since the feedback signals (952) coming from the first device towards R₃ and R₄ may be attenuated or scattered by a rib (972), R₃ and R₄ may receive a very small amplitude of the feedback signal (952). In this example, the processor of the second device may determine that the link between T₃ and the first device may also be blocked by the rib (972), since T₃ lies between R₃ and R₄. Thus, T₃ may not be selected for transmitting signals to the first device (910).

In some variations, the processor of the second device may use geometrical relations between an estimated position of the first device, positions of one or more elements of the receive array, and positions of one or more elements of the transmit array to determine driving signals for one or more transmit array elements. For example, based on the received phases, time delays and/or amplitudes of the feedback signal (952) at R₁ and R₂, and the geometrical relationship between R₁, R₂ and T₁, second device's processor may estimate an exact or an approximate phase, time delay and/or an amplitude of the feedback signal at T₁'s location (e.g., computing an average), and use it to drive T₁ with an appropriate phase, delay and/or amplitude when sending power to the first device (e.g., using time reversal).

In some variations, a first device (e.g., IMD) may be configured to generate a wireless signal (e.g., an active uplink or feedback signal, a reflection signal, a modulated backscatter signal, and the like), and a second device may comprise a first transducer array, a second transducer array, and a processor. The first transducer array may be configured to receive the wireless signal from the first device, the processor may be configured to generate first device data based on the received wireless signal, and the second transducer array may be configured to exchange one or more of wireless power and data with the first device based on the first device data. For example, the first transducer may be configured to localize (e.g., locate) the first device while the second transducer array may be configured to efficiently exchange power and/or data with the first device. In some variations, first device data may comprise one or more of a parameter related to the first device (e.g., spatial position of the first device) and a parameter related to the wireless signal generated by the first device (e.g., phase, time delay, amplitude, frequency, encoded data of the wireless signal). In some variations, the transducer elements of the first transducer array configured for localization may comprise a high impedance at the operating frequency (e.g., for increasing sensitivity to received wireless signals) compared to the second transducer array elements used for transmitting power to the first device.

In some variations, the first transducer array and the second transducer array may each comprise an ultrasound transducer array. In some variations, the second transducer array may comprise a one-dimensional linear array or a two-dimensional array. In some variations, the first transducer array may comprise at least three non-collinear transducer elements. The non-collinear transducer elements may be configured to perform triangulation of the first device based on the wireless signal generated by the first device. In some variations, the first transducer array and the second transducer array may comprise distinct transducer elements. In some variations, the first transducer array and the second transducer array may comprise at least one same (e.g., shared) transducer element. For example, the second transducer array may comprise a 1D linear array and the first transducer array may comprise the two transducer elements at the ends of the 1D linear array and a third transducer element that may not be a portion of the second transducer array and may be non-collinear with the two end transducer elements. By contrast, each element of a 1D linear array is collinear and does not allow for triangulation to locate the first device. In some variations, the first transducer array may comprise transducer elements positioned at or near the four corners of a rectangular second transducer array. In some variations, the first transducer array may comprise a subset of the second transducer array.

In some variations, the second device may comprise a third transducer array configured to transmit an interrogation signal to the first device. The wireless signal generated by the first device may comprise a feedback signal generated in response to the interrogation signal. In some variations, the third transducer array may be a subset of either the first or the second transducer arrays and may comprise one or more transducer elements. In some variations, the third transducer array may comprise distinct transducer elements from each of the first transducer array and the second transducer array. In some variations, one or more transducer elements of the second transducer array may be configured to receive the wireless signal from the first device. In some variations, the wireless signal may comprise wireless data (e.g., physiological data). In some variations, one or more transducer elements of the first transducer array and the second transducer array may be interleaved or interspersed. In some variations, the second transducer array may be configured to exchange one or more of the wireless power and data with the first device based at least in part on one or more of an interpolation and extrapolation of one or more parameters of the received wireless signal. For example, in order to determine the phases of the second transducer array elements for transmitting power (e.g., using time reversal), phases corresponding to the received wireless signal on the first transducer array elements may be interpolated or extrapolated based on the relative spatial positions of the first and second transducer array elements.

g. User Prompt

In some variations, a patient may encounter challenges in aligning an external second device on the body relative to a first device disposed inside a body of a patient since the precise location of the first device is not known. For example, a first device (e.g., an IMD) may be implanted in the body of a patient for monitoring one or more physiological parameters. The patient may be provided with a second device (e.g., an external wireless device) for wirelessly recharging and communicating with the first device.

In some variations, one or more transducer elements of the second device may be configured to transmit an interrogation signal for interrogation of a first device, as described herein. One or more transducer elements of the second device may be configured to receive a feedback signal that may be transmitted by a first device in response to the interrogation signal. In some variations, the received feedback signal may be processed by a processor of the second device to generate feedback signal data, and a user prompt may be provided for adjustment (e.g., repositioning) of the second device based on the feedback signal data.

In some variations, a user prompt comprising a location notification may be generated for instructing a user to reposition the second device based on the feedback signal. In some variations, generating the location notification may be based on an estimation of the spatial path of the first device. In some variations, a second device may be configured to measure a strength of received feedback signals on different transducer elements. The second device may be configured to determine whether the second device is aligned with the first device. For example, if transducer elements towards the left side of the second device received stronger (high amplitude) feedback signals from the first device compared to the right side of the second device, then a center of the second device may be better aligned with the first device if the second device is moved to the left. In some of these variations, a user prompt or a location notification may be generated to instruct a user to spatially adjust the second device towards the left so that the second device may be more favorably centered or positioned to power the first device, thereby improving wireless link efficiency.

In some variations, the relative power and/or voltage amplitude of the feedback signal as received by different transducer elements of the second device may be compared. Based on the results of the comparison, a user prompt may instruct a user in repositioning the second device such that elements closer to the center of the transducer array of the second device, or preferred transducer elements of the second device (e.g., transducer elements that are known to have a high efficiency or are known to be appropriately functional) may be centered or positioned closer to the first device.

In some variations, a second device may be configured to cycle through different transducer elements for transmitting an interrogation signal in any order, including a predetermined order in some variations. In some variations, a feedback signal received by three or more transducer elements of the second device may be processed to perform triangulation and determination of an approximate location of the first device relative to the second device. In some of these variations, feedback signal data may comprise data corresponding to a location of the first device (e.g., X, Y, and/or Z coordinates of the first device). In some variations, the data corresponding to the location of a first device derived from the feedback signal may be used to instruct a user through a user prompt to spatially adjust or center the second device over a first device (e.g., where the first device lies close to a central axis of the second device).

In some variations, a user prompt may be generated to instruct a user to spatially adjust the second device such that the first device is favorably positioned with respect to a predetermined set of transducer elements of the second device. For example, if different transducer elements of a second device comprise different efficiencies (e.g., electrical-to-acoustic conversion efficiency) and/or impedance characteristics, then a predetermined set of transducer elements may comprise a higher efficiency and favorable impedance profile for achieving a high overall link efficiency with a first device.

In some variations, spatial adjustment may comprise aligning an axis of the second device with a spatial path of the first device. For example, in some variations, the second device may comprise a one-dimensional (1D) linear ultrasound transducer array, and the spatial adjustment may comprise aligning one or more of an aperture and an elevation of the array with the spatial path of the first device. Aligning the elevation of the 1D array primarily along the spatial path of the first device may allow the beam width along the elevation direction to be larger (e.g., on the order of centimeters). A large enough beam width may guarantee that that first device largely remains within focus even during motion. The advantage of aligning the aperture of the 1D array primarily along the spatial path of the first device may be that the beam may be steered in that direction by phasing the array elements, thus allowing tracking of the first device in motion.

In some variations, a power notification comprising a power state of one or more of the first device and the second device may be generated. In some variations, a user may recharge the first device and/or the second device based on the power notification. In some variations, a communication notification corresponding to one or more of data received from the first device, physiological parameter data, and parameter data of one or more of the first device and the second device may be generated. The data may be provided to a health care professional and may be used to guide patient therapy.

h. Access Period

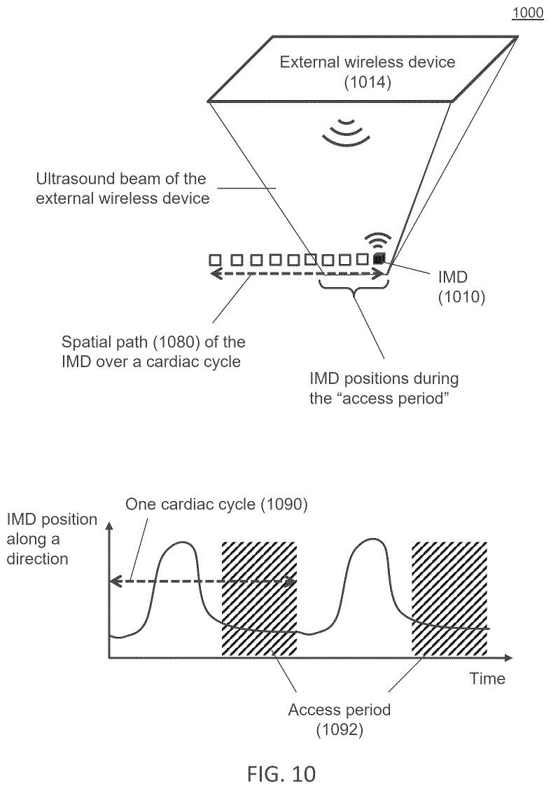

In some variations, a first device (e.g., an IMD) implanted in the heart may move and/or rotate along a spatial path (e.g., trajectory), relative to a second device (e.g., an external wireless device). For example, this could be due to the heart's pumping action and/or breathing. Due to such motion, the first device may be able to efficiently receive and transmit wireless signals from the second device only during a portion of the spatial path. The time duration during which the first device is present in the efficient portion of the spatial path is referred to as an access period and described with respect to FIG. 10.

In some variations, an access period (1092) may be short relative to a cardiac cycle (1090), as shown in FIG. 10. This may be the case for systems that may use ultrasonic energy for wireless power and data transfer because ultrasonic beams may have a millimeter scale spot size whereas the spatial path (1080) of the first device or IMD (1010) may span several centimeters. In some variations, the spatial path of a first device may be tracked by the second device and one or more transducer configurations of the second device may be determined for different positions of the first device along the spatial path. The technique may be useful to maintain the wireless link over the entire spatial path. In some variations, a different approach, as described herein, may be used to exchange wireless signals with a moving first device. In some variations, such an approach may involve exchanging wireless signals with a first device only during one or more access periods.

In some variations, an access period prediction technique may be used to find and/or predict an access period. In some variations, a first device may be configured to measure a physiological parameter (e.g., a cardiac parameter such as pressure, heart rate, and the like, and/or breathing rate, etc.) and may be configured to predict or determine an access period based on this parameter. For example, a first device implanted in the left ventricle (LV) may measure a blood pressure waveform in the LV. The duration when the pressure in the LV is relatively stable or constant may correspond to the duration when the first device is relatively stationary with respect to the second device and corresponds to an access period.

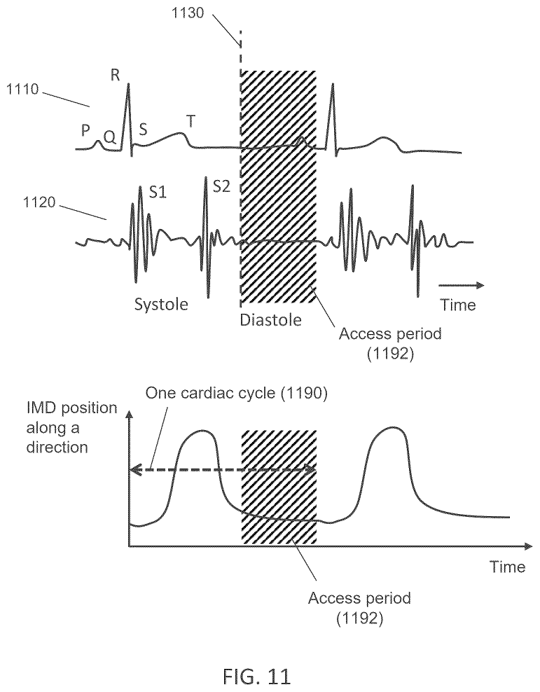

In some variations, as shown in FIG. 11, a second device may be configured to measure a physiological parameter (e.g., a cardiac parameter such as ECG, heart rate, heart sounds, blood pressure, and the like, and/or breathing rate, etc.) and may be configured to predict or determine an access period based on this parameter. For example, a second device such as an external wireless device may be configured to measure a heart rate or an ECG signal (1110). Additionally, or alternatively, the second device may measure a heard sound (1120), as shown in FIG. 11. Based on the measured ECG and/or heard sound signals, the second device may be configured to determine a start time (1130) of an access period during a cardiac cycle. For example, a start time (1130) of an access period (1192) may be set to a predetermined time delay (e.g., about 100 ms) after a detection of a heart sound (e.g., S2). In some variations, an access period may occur during a diastole.

In some variations, the access period may be predicted or determined based on a current measurement of one or more cardiac parameters or other data. In some variations, the access period may be predicted based on a previous measurement of one or more cardiac parameters or other data.

In some variations, the first device and/or the second device may be configured to interrogate another device periodically to predict or determine an access period without relying on any cardiac parameter measurement. For example, the second device may send periodic beacons to the first device and wait for the first device to acknowledge the receipt of the beacon(s). Upon receiving an acknowledgment, the second device may be configured to perform signal exchange with the first device (e.g., transfer wireless power to the first device).

i. Uplink Data Transfer

Reliable uplink data transfer from a first device (e.g., an IMD) to a second device (e.g., a wireless device) may enable accurate recovery of physiological data from the body for use in therapy. In some variations, an ultrasonic uplink signal transmitted from a first device implanted adjacent the heart may experience reflections (e.g., multipath propagation) from different tissue structures or boundaries (e.g., ribs, lungs, and the like) in one or more directions. The reflections may interfere with the uplink data received by the second device and may lead to errors in the data decoded by the second device.

In some variations, a transducer configuration of the second device (e.g., a sub-array) may be selected for receive beamforming to the location of the first device based on localization of the first device using any technique described herein. In some variations, uplink data may be transferred from a first device to a second device in one or more uplink data intervals, analogous to interval-based powering, as described herein. This may be beneficial for a first device that may move/rotate relative to the second device over time. In some variations, the second device may transmit a signal (e.g., comprising digital data bits) to the first device acknowledging the receipt of data in an uplink data interval. For example, the second device may acknowledge receipt of data based on comparison of the number of received uplink data bits to an expected number of uplink data bits corresponding to an uplink data interval. In some variations, the first device may be configured to re-transmit the corresponding data bits in a next uplink data interval when a first device did not receive an acknowledge signal from the second device until receipt is acknowledged by the second device.

In some variations, the wireless link between the first device and the second device may be disrupted (e.g., blocked) by a tissue structure such as a rib or a lung. In some of these variations, intermittent uplink data transfer may be performed in a manner analogous to intermittent powering. Uplink data may not be transferred in one or more uplink data intervals if the link gain between the first device and the second device is determined to be unfavorable. In some variations, uplink data transfer may be resumed after a predetermined time delay. For example, after a predetermined time delay, the second device may be configured to transmit an interrogation signal to the first device. A transducer configuration may be selected for efficiently receiving uplink data from the first device based upon a feedback signal received from the first device.

In some variations, digital uplink signals received by different transducer elements of the second device may be processed by a processor of the second device in order to decode the uplink signals data. For example, in some variations, at least a first transducer element of the second device may receive uplink signals with sufficient SNR for a first portion of a spatial path of a first device. At least a second transducer element of the second device may receive uplink signals with sufficient SNR for a second portion of a spatial path of the first device. In some variations, the processor of the second device may be configured to process uplink signals received by at least the first and the second transducer elements to decode data bits. The processor may combine or stitch together the data bits together in order to recover all the data bits transmitted by the first device. The technique may be extended to any number of transducer elements of the second device.

In some variations, a processor may determine when uplink data transfer from a first device to a second device is completed and/or if it is interrupted due to any reason (e.g., similar to reasons due to which power transfer may be interrupted). The first device and/or the second device may be configured appropriately such that data is not lost. In some variations, the second device may transmit a signal to the first device acknowledging receipt of data previously transmitted by the first device to the second device (e.g., in a previous uplink data interval). In some variations, a first device may erase or overwrite a data packet in memory only after it has been successfully transmitted to a second device and receipt has been acknowledged by the second device. In some variations, if a first device did not receive an acknowledgment from the second device for a previously transmitted data packet, it may retain that data packet in memory and retransmit until an acknowledgment signal is received from the second device.

In some variations, as an example, a first device may track of one or more pointers (e.g., memory address pointers) in its memory. The pointers may encode an address or a start address of a data block that may have already been transmitted to the second device and/or acknowledged by the second device, and/or an address or a start address of a data block that may have not yet been transmitted to the second device. Based on an acknowledgement received from the second device, the first device may update one or more of the pointers. In some variations, a first device may be configured to uplink the value of the one or more pointers to the second device using a feedback signal or uplink data.

In some variations, a second device may notify a user based on a user prompt if uplink data transfer is interrupted or a percentage of data transfer that may have been completed up to a predetermined point in time. For example, a difference between the two pointers described herein (e.g., one pointer pointing to transmitted/acknowledged data block and another pointer pointing to a data block yet to be transmitted to the second device) may indicate a percentage of data successfully received by the second device.

In some variations, a first device may be configured to stop transmitting uplink data to the second device. For example, a feedback signal from a first device may encode the values of pointers to its memory, as described herein, based on which the second device may send a command to the first device to stop transmitting uplink data if all of the data in the first device memory is successfully read by the second device. In some variations, a first device may automatically stop transmitting uplink data to the second device if the stored data is transmitted and acknowledged by the second device.

In some variations, a second device may generate a user prompt including, but not limited to, the percentage of first device's data successfully received by the second device, whether uplink data transfer is completed, whether uplink data transfer is interrupted due to any reason, combinations thereof, and the like. A user may take an action based on a user prompt, such as recharging the second device's battery if the second device's battery is running low, re-positioning the second device if the second device is displaced, turning off and removing the second device if uplink data transfer is complete, combinations thereof, and the like.

j. First Device Transducer Selection

In some variations, a first device (e.g., an IMD) may comprise a plurality of transducer elements (e.g., a plurality of ultrasonic transducer elements) that may be selected individually for different operations including one or more of transmitting uplink signals (e.g., feedback signal, data), receiving power, receiving downlink signals (e.g., downlink data, commands), combinations thereof, and the like. First device transducer selection may enable robust, error-free data communication with a second device (e.g., external wireless device, wireless device).

In some variations, a first device (e.g., IMD) may comprise a plurality of transducers configured to receive a downlink signal. A second device (e.g., a wireless device) may be configured to transmit the downlink signal. One or more of the plurality of transducers may be configured to exchange one or more of wireless power and data with the second device based on the received downlink signal. In some variations, selection of one or more transducer elements of the first device may be performed by the first device by processing one or more received downlink signals from the second device, including one or more of an interrogation signal, power, downlink commands, other downlink signals, and the like. In some variations, selection of one or more transducer elements of the first device may be performed by the second device by processing one or more received uplink signals from the first device, including one or more of a feedback signal, uplink data, and the like, and communicating this data to the first device using a downlink command (e.g., the second device may program a first device to use one or more specific transducer elements for an operation).

In some variations, selection of one or more transducer elements of the first device for an operation may be based upon determining one or more transducer elements that received the highest power or voltage, or a power or voltage above a predetermined threshold, from a downlink signal such as an interrogation signal. The selection of transducer elements of a first device may be analogous to the selection of transducer elements of the second device based on a received feedback signal described herein.

In some variations, only one transducer element of the first device may be selected for an operation (e.g., receiving power, receiving downlink signals, transmitting uplink signals). For example, a processor of a first device may be configured to process interrogation signals received by each of the transducer elements, determine the transducer element that received the highest power or voltage from the interrogation signal, and select that transducer element for transmitting an uplink signal such as uplink data. In some variations, the first device may comprise a multiplexer and/or a demultiplexer circuit comprising switches or a switch network that may be coupled to the transducer elements. In some variations, the switches may be configured to connect only the selected transducer element to an uplink data transmitter in order to transmit an uplink signal only using the selected transducer element. This may allow selection of the transducer element that has the best link gain with the second device, leading to a robust data link with a high SNR and low bit error rate. In some variations, one or more of the plurality of transducers of the first device may be configured to exchange wireless data with the second device at a first frequency different from a second frequency of the received wireless power.

In some variations, the first device may further comprise a processor. In some variations, the processor may be configured to select one or more of the plurality of transducers configured to exchange one or more of the wireless power and data with the second device based on the received downlink signal. In some variations, the processor may be configured to update the selection periodically based on one or more of the received downlink signals. In some variations, the processor may be configured to calculate a received signal strength of the downlink signal for one or more of the plurality of transducers and compare the received signal strengths of one or more of the plurality of transducers against each other. In some variations, the processor may be configured to select one or more of the plurality of transducers corresponding to the received signal strength above a predetermined threshold, for exchanging one or more of the wireless power and data with the second device. In some variations, the processor may be configured to select one transducer corresponding to a maximum received signal strength for transmitting an uplink signal to the second device. In some variations, the processor may be configured to decode one or more downlink commands based on the downlink signal. In some variations, the processor may be configured to select one or more transducers for exchanging one or more of the wireless power and data with the second device based on decoding one or more of the downlink commands.

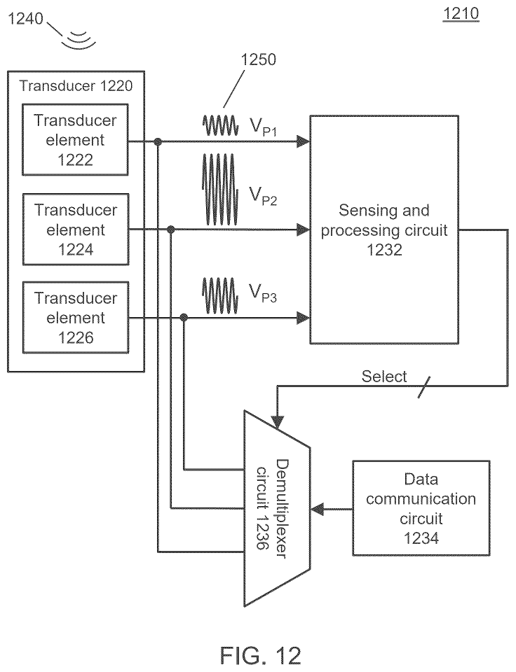

FIG. 12 depicts a block diagram of a first device (1210) in accordance with an illustrative variation of this system. The transducer (1220) of the first device (1210) may comprise a plurality of transducer elements (1222, 1224, 1226). The first device (1210) may be configured to receive a downlink signal (1240) from a second device (not shown), such as an interrogation signal, power, and the like. Each transducer elements (1222, 1224, 1226) may receive a different voltage or power from the downlink signal (e.g., due to the orientation of a transducer element relative to the second device). Voltages (1250) received by the plurality of transducer elements (1222, 1224, 1226) may be compared. As shown in FIG. 12, one transducer element (1224) may receive the highest voltage amplitude compared to the other transducer elements (1222, 1226). The processor of the first device (1210) may comprise a sensing and processing circuit (1232) that may compare the voltage amplitudes (V_{P1}, V_{P2}, V_{P3}, each of which may be an open-circuit voltage) received by the plurality of transducer elements to generate a select signal (comprising one or more digital control bits) provided to a demultiplexer circuit (1236). The input to the demultiplexer circuit (1236) may be an output of a data communication circuit (1234) such as the output of a power amplifier circuit for uplink data transmission. The demultiplexer circuit (1236) may comprise one or more switches or a switch network that may be configured by the select signal to connect one transducer element (1224) to the input of the demultiplexer circuit (1236), and not the other transducer elements (1222, 1226) for uplink data transmission. It should be appreciated that the specific circuit implementation of the demultiplexer circuit discussed here (using switches) is an example, and other variations may be possible, such as using frequency-based selection (e.g., using filters), amplitude-based selection, circulators, diodes or passive devices, and the like. Additionally or alternatively, in some variations where a transducer element may be selected for an operation such as receiving power or a downlink signal (e.g., data, commands), the first device (1210) may comprise a multiplexer circuit (not shown) which may be analogous to the demultiplexer circuit for uplink data transmission. The multiplexer circuit may be configured or controlled by the sensing and processing circuit (1232).

In some variations, a selected transducer element may be selected for an operation such as receiving power from the second device by configuring or connecting only the selected transducer element to a power circuit such as a rectifier or an AC-DC converter. For example, switches may be implemented between each transducer element and a power recovery circuit (e.g., rectifier). During power recovery, one or more switches between the selected transducer element and the power recovery circuit may be turned ON, and one or more switches between the other transducer elements and the power recovery circuit may be turned OFF.

In some variations, a selected transducer element may be selected for an operation such as receiving downlink signals (e.g., downlink data, commands, etc.). For example, the processor of a first device may process downlink signals received by only the selected transducer element (e.g., the transducer element that receives the highest voltage or power from an interrogation signal or a downlink signal) in order to decode downlink data and/or commands.

In some variations, more than one transducer element of the first device may be selected for an operation. For example, in some variations, a processor of the first device may be configured to measure relative phases or delays of the downlink signals (e.g., an interrogation signal) received by its transducer elements. The measured data may be used to drive the transducer elements with appropriate phases, delays and/or amplitudes for transmitting an uplink signal. This may be beneficial to avoid undesired signal cancellation (e.g., null lobes) while transmitting signals using more than one transducer element. In some variations, a first device may transmit an uplink signal on a plurality of transducer elements (e.g., all transducer elements) with the same phase (to minimize design complexity) or with appropriate phases when it is known or predictable that undesired signal cancellation may not occur. As another example, in some variations, more than one transducer element of the first device may be configured to receive power from the second device by using techniques such as power or DC combining. In some variations, each transducer element of the first device may be selected for an operation such as receiving power, receiving downlink signals, and/or transmitting uplink signals.

In some variations, in addition to selecting one or more transducer elements of a first device for an operation, the drive signals, or the way signals are received from those transducer elements, may also be configured. For example, a first device and/or a second device may determine one or more of frequency, phase, delay, amplitude, power of the uplink signal, combinations thereof, and the like, for transmitting an uplink signal, in addition to selecting one or more transducer elements of a first device.

In some variations, selection of one or more transducer elements of the first device (and/or their corresponding drive signals, or the way in which signals are received from them) may be performed at any time during the execution of any of the methods described herein. For example, selection of one or more transducer elements of the first device, and/or determination of their drive signals (e.g., frequency, amplitude, etc.), may be performed at the beginning of every uplink data interval during interval-based uplink data transfer.

k. Single Transducer First Device

In some applications, a first device (e.g., an IMD) may comprise a single transducer (e.g., a single ultrasound transducer) configured to perform different wireless functions such as receiving interrogation signals, receiving power, receiving downlink data signals and transmitting uplink signals. This may allow miniaturization of the first device. The first device may further comprise a multiplexer circuit configured to decouple the various signals received/transmitted using the single transducer. In some variations, the multiplexer circuit may be controlled by a controller circuit which in some variations may be a component of a processor of the first device. The multiplexer circuit may be configured for different operations such as receiving interrogation signals and power/downlink data from the second device and transmitting uplink signals to the second device. While solutions presented below are discussed in terms of a multiplexer circuit comprising switches, other variations of a multiplexer circuit as described before may be applicable here.

In some variations, a first device may comprise a wake-up receiver circuit configured to detect interrogation signals received by the first device from a second device. The wake-up receiver circuit may comprise one or more of an envelope detector circuit, a code detector or a decoder circuit, combinations thereof, and the like. In some variations, the wake-up receiver circuit may be coupled to the multiplexer circuit. The multiplexer circuit may be controlled by the controller circuit to couple (e.g., connect using switches) the wake-up receiver circuit to the transducer in a default mode of operation. For example, a default mode of operation may be a mode of the first device before receiving a predetermined interrogation signal. For example, in the default mode, the first device may be autonomously performing a function such as sensing, or may not be performing any function (i.e., may be in a sleep state). This configuration of the multiplexer circuit may allow the first device to be ready (e.g., on standby) for detecting any ad-hoc interrogation signals sent by the second device.

In some variations, the multiplexer circuit may be configured to keep a power recovery circuit (e.g., a rectifier) disconnected from the transducer in the default mode. When an interrogation signal is received, the wake-up receiver circuit may detect the interrogation signal (e.g., via envelope detection) and generate a wake-up signal that may be provided to the controller circuit. In some variations, the wake-up receiver circuit may generate the wake-up signal when the interrogation signal amplitude crosses a threshold, or has a specific code or embedded command, or both.

In some variations, the controller circuit may control the multiplexer circuit to connect the power recovery circuit to the transducer upon receiving the wake-up signal. This may allow the first device to recover power from power signals subsequently sent by the second device to the first device.

In some variations, the controller circuit may control the multiplexer circuit to couple the transducer to a transmitter circuit upon receiving the wake-up signal in order to transmit uplink signals (e.g., feedback signals) to the second device. For example, the first device may be configured to sample the amplitude of the received interrogation signal, digitize it and then send this digitized amplitude to the second device as a feedback signal. Additionally or alternatively, the feedback signal may include one or more analog pulses, and/or one or more digital bits, for acknowledging to the second device the receipt of the interrogation signal by the first device. Upon receiving one or more of the feedback signals, the second device may perform localization of the first device and/or select a transducer configuration for establishing an efficient link with the first device. In some variations, the second device may then transmit power to the first device. In some variations, the controller circuit of the first device may control the multiplexer circuit to decouple the transmitter circuit from the transducer upon transmitting the feedback signals. In some variations, simultaneously or subsequent to decoupling the transmitter circuit, the controller circuit may control the multiplexer circuit to couple the transducer with the wake-up receiver circuit (i.e., going back to default mode to wait for another interrogation signal from the second device), or with the power recovery circuit (to recover power sent by the second device).

In some variations, the second device may send a command or a code to the first device to indicate that it will be transmitting power signals next after receiving the feedback signals from the first device. In some variations, the wake-up receiver circuit may detect such a code, provide a corresponding signal to the controller circuit. The controller circuit may then couple the power recovery circuit to the transducer to configure the first device for receiving power.

In some variations, the controller circuit may control the multiplexer circuit to disconnect the transducer from the power recovery circuit after the end of powering. The controller circuit may then re-connect the transducer to the wake-up receiver circuit (i.e., going back to default mode). In some variations, the controller circuit may control the multiplexer circuit to decouple the transducer from the power recovery circuit after the end of powering. The transducer may then be coupled to the transmitter circuit to transmit feedback signals to the second device (e.g., conveying an energy state of the first device). The transducer may then be coupled to the wake-up receiver circuit (i.e., going back to default mode). In some variations, the second device may convey an end of powering to the first device. In some variations, the first device may automatically perform these steps after a falling edge of the received power signal (e.g., based on a timeout).

In some variations, the multiplexer circuit may be configured to keep both the wake-up receiver circuit and the power recovery circuit coupled to the transducer in the default mode. This may allow the first device to recover power and/or charge its energy storage device from an interrogation signal.

l. Downlink Signal Amplitude Detection

In some variations, the second device may use first device data to tune the transmitted power to the first device and/or to select a transducer configuration for efficiently exchanging wireless power/data with the first device. In some variations, a first device (e.g., an IMD) may be configured to detect an amplitude of a received downlink signal, such as an interrogation signal, a power signal, downlink data, and the like. For example, a first device may be configured to detect an amplitude of a downlink signal (e.g., peak voltage on the ultrasound transducer) received from a second device (e.g., a wireless device). The amplitude may be compared to a threshold or digitized. A feedback signal comprising the digitized amplitude or comparison result may be transmitted to the second device.

In some variations, the first device may comprise a first envelope detector circuit coupled to the ultrasound transducer of the first device to generate a first output voltage corresponding to a voltage amplitude of the downlink signal received by the ultrasound transducer. The first envelope detector circuit may comprise a peak detector circuit, a rectifier, and the like. In some variations, the first output voltage may be proportional or equal to an amplitude, or a maximum value of the voltage received by the ultrasound transducer of the first device. For example, the first envelope detector circuit may comprise a diode in series with a parallel combination of a capacitor (C₁) and a resistor (R₁). In some variations, R₁ may be very large or infinity (i.e., no resistor).

In some variations, the time or a sampling trigger may be determined to enable the first device to sample the first output voltage. In some variations, the first device may be configured to sample the first output voltage of the first envelope detector circuit in order to estimate the voltage amplitude received by the ultrasound transducer.

In some variations, the first device may comprise a second envelope detector circuit coupled to the first device's ultrasound transducer to determine a sampling trigger. The second envelope detector circuit may be configured to generate a second output voltage corresponding to the voltage amplitude of the downlink signal received by the ultrasound transducer. In some variations, the second envelope detector circuit may be configured with a faster response time than the first envelope detector circuit. For example, the second envelope detector circuit may comprise a diode in series with a parallel combination of a capacitor C₂ and a resistor R₂, such that the output time constant C₂R₂ is smaller (e.g., more than 10 times smaller) than the output time constant C₁R₁ of the first envelope detector circuit. Thus, the second output voltage may drop faster than the first output voltage upon the falling edge of the downlink signal. In some variations, the falling transition of the second output voltage may be directly used as, or processed (e.g., using one or more inverters) to generate a sampling trigger for sampling the first output voltage. The sampled first output voltage may be representative of the downlink signal amplitude received by the first device's ultrasound transducer.

In some variations, a timer or a delay generator circuit may be configured to sample the first output voltage for a predetermined time duration after the receipt (or rising edge) of the downlink signal by the first device. In some of these variations, a second envelope detector circuit is not needed.

m. First Device Mode Interrogation

For proper functioning of the system, a first device may be configured to detect an interrogation event and respond appropriately to the interrogation. In some variations, a first device (e.g., an IMD) may operate by default in a first mode. For example, the first mode may be a sensing mode where the first device may be configured to sense a physiological parameter periodically. The first mode may be a sleep mode where the first device may be dormant and waiting for an interrogation signal from the second device (e.g., a wireless device). During a first mode, a first device may be interrogated by a second device for recharging the first device's energy storage device (e.g., battery) and/or for exchanging data.

In some variations, a first device may comprise a wake-up receiver circuit configured to monitor ultrasound signals received by device's one or more ultrasonic transducers of the first device. In some variations, the wake-up receiver circuit may be configured to detect a signature or a code encoded by the second device in the interrogation signal to determine the purpose of interrogation (e.g., second device intends to recharge the first device, or the second device intends to recover data stored in the first device, and the like). In some examples, a wake-up receiver circuit may comprise an envelope detector, a comparator and a decoder circuit.

In some variations, the first device may respond to the interrogation signal of the second device using a feedback signal. Additionally, or alternatively, a handshake of one of more signals may be exchanged between the second device and the first device. In some variations, the first device may configure itself to stop the first mode and start a second mode of operation (i.e., the interrogation signal may cause the first device to override its first mode of operation and enter a second mode) upon detection of the interrogation signal. For example, the second mode may comprise a wireless uplink mode where the first device may configure itself to transmit its stored data using at least one uplink signal. In some variations, the first device may configure itself to operate in a second mode in addition to the first mode upon detection of the interrogation signal. For example, the second mode may be a wireless uplink mode and the first mode may be a sensing mode. The first device may be configured to time multiplex functions corresponding to the two modes, or perform them simultaneously. In some variations, the uplink data transfer may occur over one or more intervals (e.g., interval-based data transfer, similar to interval-based powering), which may aid data recovery from a moving first device. After the data transfer is complete, the first device may automatically return to the first mode. In some variations, the second device may send a command to the first device using a downlink signal to configure the first device to go back into the first mode before or after completion of uplink data transfer.

In some variations, additionally or alternatively, based on one or more feedback signals, the second device may generate a user prompt (e.g., feedback) to instruct a user to manually adjust the second device, if doing so may improve exchange of signals (power, data) in the second mode. In some variations, if no feedback signals are received upon interrogation, or based on one or more received feedback signals, the second device may generate a user prompt instructing a user to consult a health care professional.

### F. Gauge Pressure Estimation

In some variations, a first device (e.g., IMD) may be configured to directly measure absolute pressure corresponding to a blood pressure, such as in a heart chamber or a blood vessel, and the like. For example, an absolute pressure value may be referenced to a vacuum or a known pressure. Gauge pressure may be estimated from absolute pressure measurements. Gauge pressure may be referenced to the ambient air pressure or atmospheric pressure. Gauge pressure may be predetermined by the absolute pressure measured by the first device minus the atmospheric pressure. Thus, in some variations, in addition to an absolute pressure measurement by a first device, a corresponding measurement of atmospheric pressure may enable accurate determination of gauge pressure for disease monitoring and therapy. Systems, device, and methods are described herein to estimate gauge pressure from the absolute pressure measured.

In some variations, left ventricular end-diastolic pressure (LVEDP) may be measured to monitor the progression of a patient's heart failure. LVEDP values (which may be gauge pressure values) may typically be low, for example, lying in the range of about 0 mmHg to about 40 mmHg. Atmospheric or ambient pressure at a patient's location may be variable and may depend on a number of factors including, but not limited to, weather conditions, ambient temperature, whether the patient is on an airplane, altitude of the patient relative to sea level, combinations thereof, and the like. For example, atmospheric pressure at sea level may be about 760 mmHg, whereas may be about 720 mmHg at an altitude of about 500 m (a difference, compared to sea level, of 40 mmHg which is on the order of LVEDP). Thus, accurate determination of atmospheric pressure may be important for the accurate estimation of LVEDP in this example, and blood pressure, in general. In some variations, a second device (e.g., an external wireless device) may be configured to measure atmospheric pressure. In some variations, a first device may measure absolute pressure inside the body independently of second device), i.e., without being simultaneously interrogated or commanded by a second device to measure pressure. For example, an implanted first device may be configured to continuously monitor blood pressure throughout a day (e.g., the first device may be powered by a battery). The second device may not be placed on a patient's chest or may not be carried by a patient at all times.

In some variations, a second device or any external device may be configured to measure atmospheric pressure. For example, in some variations, a second device configured to power and/or communicate with a first device may also comprise a barometric sensor or a pressure sensor configured to measure atmospheric pressure. In some variations, a barometric sensor may be located on an external device such as a phone, a smartwatch, a tablet, and the like. Additionally, or alternatively, an application such as a mobile app may be configured to run on the external device to record and/or process atmospheric pressure values. In some variations, a second device or any external device may obtain atmospheric pressure values from the internet or from another device. In some variations, a patient may carry with them a second device or an external device configured to obtain, record, and/or process atmospheric/ambient pressure values. For example, a second device configured to measure atmospheric pressure may be attached to a patient's cell phone, or may be placed in a purse or a bag that may be carried by the patient with them. In some variations, a second device may be worn on the body (e.g., in the form of a patch, a sleeve, a strap, a belt, and the like).

In some variations, absolute pressure measurements measured by a first device may be post-processed by a second device (e.g., software processing) to estimate gauge pressure. Post-processing may identify features or signatures of atmospheric pressure, or atmospheric pressure variations, in the absolute pressure data. These features or variations may be subtracted to determine gauge pressure. For example, post-processing may comprise using low or high pass filters to eliminate atmospheric pressure variations from the absolute pressure data. In some variations, atmospheric pressure may change at a slower rate compared to blood pressure. In some variations, if a significant deviation is detected in the absolute pressure, software processing may attribute it to a change in atmospheric pressure. In some variations, post-processing may be based on data such as a patient's travel history (e.g., travel times/locations) and the like, to estimate atmospheric pressure and subsequent gauge pressure estimation.

In some variations, a first device may be configured to measure or estimate atmospheric pressure. In some variations, a first device may be configured to measure or determine gauge pressure and/or a pressure correlated with gauge pressure, by measuring both an absolute pressure of interest (e.g., absolute pressure of blood in the LV, any physiological pressure change in a tissue or organ of interest) and an estimate or proxy of the atmospheric pressure. For example, in some variations, a first device may comprise a first pressure transducer located in the lumen of a heart chamber or a blood vessel. The first pressure transducer may be directly affected by or may directly sense blood pressure. The first device may further comprise a second pressure transducer positioned or situated at a location (e.g., inside a heart wall or a septum) where it may not be directly affected by or may not directly sense blood pressure. In some variations, processing of the signals measured by the first and second pressure transducers (e.g., subtracting one from the other) may provide the one or more of the desired gauge pressure, a proxy for the gauge pressure, or a pressure that may be correlated with gauge pressure. In some variations, a single pressure transducer may comprise a first portion located inside the lumen of a heart chamber or a blood vessel and a second portion situated at a location (e.g., inside a heart wall or a septum) where it may not be directly affected by or may not directly sense blood pressure. In some variations, a different first device (e.g., another IMD) may be configured to measure atmospheric pressure. For example, a first device may be implanted just under the skin (e.g., a few mm under the skin of the arm) to measure atmospheric pressure or a reference pressure that may be used to determine gauge pressure from the absolute blood pressure measured by another first device.

In some variations, time synchronization between the measurements of absolute blood pressure and atmospheric pressure may be used to accurately subtract atmospheric pressure data from absolute blood pressure data to estimate gauge pressure. For example, atmospheric pressure measured by a different device (e.g., by a second device, any external device, or a different first device) may be compared to the absolute blood pressure measurements of the first device disposed in the body. In some variations, a first device and a second device may be time synchronized at one point in time, after which, both devices may record pressure data at a fixed rate. For example, a second device and/or an external device (e.g., a phone) and a first device may be synchronized to 9:00 a.m. on a predetermined day, after which the first device may record one or more absolute blood pressure values about every 5 minutes. The second device and/or the external device (e.g., phone) may record one or more atmospheric pressure values about every 5 minutes. The first device may store absolute blood pressure values in its memory. Additionally or alternatively, the first device may store one or more time points (e.g., a time, a date, a value corresponding to the first device's counter or timer, and the like) corresponding to the one or more of the stored absolute blood pressure values in memory. After a subsequent time period (e.g., after one or more hours, days, and the like), the second device may download the absolute blood pressure data from the first device (e.g., via uplink data transfer). The second device may align or synchronize the time points of the first device's absolute blood pressure data and the time points of the second device's (or an external device's) atmospheric pressure data, and then perform subtraction to determine gauge pressure values at one or more time points.

### G. Ultrasound Imaging

In some variations, a first device (e.g., IMD) may be configured to sense (e.g., measure) one or more physiological parameters (e.g., blood pressure) in conjunction with an imaging technique (e.g., transthoracic echocardiography or TTE) for measuring one or more physiological parameters (e.g., blood flow). Measurements using a plurality of modalities may improve diagnosis, monitoring, and treatment (e.g., appropriate adjustment of medication) of a patient. For example, a first device implanted in a heart chamber or a blood vessel may be configured to measure blood pressure and TTE may measure blood flow or velocity to assess one or more structures or motion of the heart or heart chambers of a patient (e.g., flow or velocity through the left ventricular outflow tract, LV contractions or LV wall motion, prosthetic valve leaflet thickness/motion, and the like). In some variations, TTE may be one of the imaging techniques configured to diagnose and/or monitor patients with heart failure, valvular heart disease, prosthetic valve dysfunctions, combinations thereof, and the like. TTE imaging in conjunction with sensing by the first device may generate interference. For example, TTE imaging may couple ultrasonic signals to a pressure transducer of a first device so as to corrupt the sensed pressure data. Similarly, a first device transmitting ultrasonic signals (e.g., for uplink data transfer) during a TTE procedure (e.g., during CW Doppler imaging for blood velocity measurement) may interfere with ultrasonic signals, leading to errors or corruption of the TTE data or images.

In some variations, an ultrasound signal (e.g., interrogation signal, downlink data, commands, and the like) transmitted by a second device to a first device may encode a specific code (e.g., an ID, a command). The first device may respond to the received signal upon detecting (e.g., decoding) such a code. Signal encoding may reduce erroneously generated signal transmission (e.g., feedback signal, ultrasound response signal) due to due to imaging procedures such as TTE.

In some variations, a first device may be configured to operate in a first mode such as a sensing mode during a time period when ultrasonic imaging such as TTE is not being performed. In the sensing mode, the first device may be configured to periodically monitor or sample a physiological parameter such as blood pressure and store physiological parameter data in its memory. For example, a first device in the sensing mode may be configured to perform one or more of sampling blood pressure, processing the sampled pressure values (e.g., computing peak pressure, mean pressure, ignoring certain values, combinations thereof, and the like), storing processed data (or physiological parameter data) in its memory, receiving wireless signals (e.g., interrogation signal, power, downlink data, commands, and the like) from a second device, transmitting wireless signals (e.g., feedback signal, uplink data) to a second device, combinations thereof, and the like. In some variations, prior to initiating an ultrasonic imaging procedure such as TTE, a second device may configure the first device (e.g., via a downlink command) to operate in a second mode (e.g., waveform storage mode) during a time period when ultrasonic imaging such as TTE is performed. For example, the first device in the second mode may sample pressure multiple times over one or more cardiac cycles and store pressure datapoints in its memory. The samples may be used for patient diagnosis and/or monitoring. For example, the samples may be processed to plot a waveform of pressure over one or more cardiac cycles. In some variations, the techniques described herein for mitigating coupling between an ultrasound signal and a pressure transducer may be applied in a similar manner for mitigating coupling between TTE signals and a pressure transducer of a first device.

In some variations, a first device comprising limited memory capacity may transmit data to a second device prior to operating in a waveform storage mode. For example, a second device may download data (e.g., data captured during the sensing mode) from the first device's memory (e.g., via uplink data transfer) to free up some or all memory space (e.g., the first device may erase its memory after transferring data to the second device and receiving a signal from the second device acknowledging data receipt) prior to operating in the waveform storage mode. This may allow one or more pressure waveforms to be stored in the first device.

In some variations, ultrasonic imaging such as TTE may be performed to measure one or more physiological parameters after a first device is configured in the second mode (e.g., waveform storage mode). For example, a first device may be configured to operate in the second mode to sense and store pressure waveforms (e.g., LV pressure waveform) over one or more cardiac cycles. Then, TTE may be performed to measure blood velocity or flow (e.g., in the left ventricular outflow tract or LVOT). In some variations, TTE may use pulse wave (PW) Doppler imaging or continuous wave (CW) Doppler imaging for measuring velocity or flow. In some variations, a first device may continue to overwrite its memory with data corresponding to pressure waveforms over one or more cardiac cycles, or may automatically transition into another mode (e.g., sleep mode) when the memory reaches capacity.

In some variations, a first device may be configured to uplink data (e.g., sensed pressure waveform data) to a second device in real-time while TTE measures a physiological parameter (e.g., flow). In some variations, a first device may be configured to sense the presence of TTE signals (e.g., pulses corresponding to PW Doppler) and sense pressure and/or transmit an uplink signal to a second device when the TTE signal is not present to avoid signal interference. For example, a first device may be configured to sense pulses corresponding to PW Doppler and may optionally determine a repetition rate or pulse repetition frequency (PRF) of PW Doppler pulses from the detection of pulses. The first device may be configured to sense pressure and/or transmit an uplink signal to a second device during the gap or OFF time between consecutive PW Doppler pulses. Additionally, or alternatively, the first device may be configured to estimate a distance of between a focal spot of PW Doppler in tissue and the TTE source based on the measured PRF.

After completion of one or more TTE procedures, a second device may be configured to download (e.g., via uplink data transfer) the data stored by the first device in the second mode (e.g., LV pressure waveform data). In this manner, a health care professional (e.g., cardiologist) may gain access to data measured by a first device (e.g., pressure waveforms over one or more cardiac cycles) and by TTE (e.g., flow over one or more cardiac cycles) during a single checkup procedure. In some variations, at the end of an ultrasonic imaging procedure such as TTE during a checkup procedure, a second device may be used to configure a first device (e.g., via a downlink command) to operate in the first mode (e.g., sensing mode). In some variations, a first device may transition into the first mode (e.g., sensing mode) on its own (e.g., after a predetermined time out).

In some variations, ultrasonic imaging such as TTE may be performed after configuring a first device to operate in the second mode (e.g., waveform storage mode) and downloading data corresponding to the second mode (e.g., LV pressure waveforms over one or more cardiac cycles) using a second device. In some variations, ultrasonic imaging such as TTE may be performed while a first device is operating in the first mode (e.g., before configuring the first device to operate in the second mode).

### H. Long-Term Parameter Tracking

In some variations, long-term parameter tracking may be used to identify changes in either patient physiology or of the wireless system. For example, tracking one or more parameters (e.g., characteristics, properties, features) of the wireless link (e.g., link gain), a parameter related to the wireless system, a parameter related to the physiology or disease of the patient, combinations thereof, and the like,) over a predetermined time period (e.g., hours, days, weeks, months, years) may enable identification of a change and/or a failure or degradation mode of the wireless link and/or the wireless system and/or the patient's physiology.

In some variations, long-term tracking may be useful as a “seed” or a starting point for a second device (e.g., external wireless device) configured to power and/or communicate with a first device (e.g., IMD). For example, in some variations, a transducer element of a second device may transmit an interrogation signal, a transmit power, and the like, using previously stored values, or an extrapolation of previously stored values, based on long-term tracking. In some variations, long-term tracking may aid diagnosis or monitoring of a patient's condition. For example, long-term tracking of the position or motion of a first device attached to an LV wall may generate useful data corresponding to the motion of the LV wall itself, which may be used to monitor the progression of heart failure in a patient.

As used herein, a parameter (e.g., property, feature) tracked (e.g., measured, sensed, estimated, identified, detected) over the long term may be referred to as a tracked parameter. In some variations, a tracked parameter may include, but is not limited to, wireless link gain between a first device and a second device, transmit power of the second device, transmit frequency of the second device, one or more parameters of the transducer configuration of the second device configured to exchange wireless power and/or data with the first device, the transducer element(s) of the first device having the highest link gain with the second device, energy state of the first device, a battery life of the first device, a parameter related to a pressure transducer of the first device (e.g., sensitivity, drift), a parameter related to an ultrasound transducer of the first device (e.g., impedance, efficiency), transmit frequency of the first device, transmit power of the first device (e.g., for uplink or feedback signals), one or more positions and/or orientations (e.g., angle relative a second device's axis) of the first device, a parameter related to any system, device, or method described herein, a parameter related to a body (e.g., a physiological parameter), combinations thereof, and the like.

In some variations, a tracked parameter may include, but is not limited to, one or more of tissue encapsulation of the first device (e.g., an IMD), movement, drift or rotation of the first device, degradation of the first device, patient gaining/losing fat, fluid retention in a patient's lungs, motion of a patient's heart wall or chamber or location in or near to which a first device may have been implanted, other failure or degradation modes of the link/system, progression of a patient's disease (e.g., heart failure monitoring), combinations thereof, and the like.

In some variations, an external device (e.g., second device, another second device) may be configured to store one or more tracked parameters in memory. For example, in some variations, a second device or another external device may be configured to track one or more of a link gain, transmit power, time of flight of ultrasound signals (e.g., from the second device to the first device and back to the second device), transducer configuration, phases and frequency required for efficiently powering a first device. In some variations, the second device may be configured to compare the required transmit power in a predetermined powering session to the transmit power required in one or more previous powering sessions (e.g., which may have been done weeks or months ago). Changes in one or more tracked parameters may indicate one or more of rotation of a first device, patient gaining/losing fat or weight, encapsulation of the first device, any degradation of the first device and/or the second device, changes in the physiological state of a patient (e.g., fluid retention), combinations thereof, and the like.

In some variations, upon detecting a change exceeding a predetermined threshold in one or more tracked parameters, a second device may generate a user prompt as described herein. A user prompt may comprise one or more of instructing a user on how to correctly operate the second device (e.g., if it is determined that the change may be due to incorrect usage of the second device), alerting a patient to contact a health care professional (e.g., if a significant degradation, rotation or drift of the first device is detected, if changes in fluid retention in the lungs is detected), alerting the doctor directly, combinations thereof, and the like. In some variations, a health care professional may reposition and/or adjust a first device and/or implant another first device if there is a predetermined rotation, drift, or degradation of the first device.

### II. Methods

Described herein are methods for establishing a reliable and/or efficient wireless link (e.g., for power transfer and/or data communication) between a first device (e.g., an IMD) and a second device (e.g., an external wireless device, a wireless device), using the systems and devices described herein. In some variations, the first device may be implanted in a patient's body for performing one or more functions such as monitoring physiological signals or parameters (e.g., blood pressure, blood flow, neural action potentials, etc.) and stimulating tissue (e.g., nerve, muscle, etc.). In some variations, the first device may be configured to receive wireless power from a second device. In some variations, the first device may be configured to wirelessly communicate data and/or commands bi-directionally with a second device. In such systems, establishing a reliable and/or efficient wireless link for power and/or data transfer may be important for minimizing energy dissipation of the IMD and the external wireless device, minimizing tissue heating and achieving error-free data transfer for accurate disease monitoring and therapy. A position of an implantable IMD may not be initially known to a wireless device before establishing a power and/or data link with the IMD. The IMD may be implanted in a complex non-homogeneous tissue medium, making it challenging to establish an efficient and reliable wireless link. Thus, it may be important to locate/track the IMD and/or find a transducer configuration, as described in detail herein.

Methods described here may comprise one or more of the following steps, including, but not limited to, sending an interrogation signal from a wireless device (using a sub-array), receiving a feedback signal at the wireless device, processing the feedback signal (using a processor of the wireless device) to generate feedback signal data, and finding/determining a transducer configuration based on feedback signal data. Methods are also provided herein for adequate positioning of a wireless device on a patient's body for establishing a reliable wireless link with an implanted device, noise reduction for accurate sensing, and estimation of a heart rate of patients.

In some variations, a method of positioning a wireless device on the body may comprise generating a user prompt corresponding to a desired location on the body, and orienting the wireless device according to one or more of an orientation feature and an orientation signal of the wireless device. This may be beneficial for at-home monitoring in order to guide inexperienced users such as patients for correctly positioning the wireless device for disease monitoring and/or therapy. In some variations, a method of positioning a wireless device on the body may comprise measuring a parameter of one or more of the wireless device and the body, estimating a position of the wireless device on the body based on the measured parameter, and generating a user prompt corresponding to the estimated position of the wireless device on the body. Such a method may provide ease of use to patients by enabling automatic detection of the positioning of the wireless device on the body, and a user prompt that may help guide users to establish a reliable wireless link with the IMD.

In some variations, a method of coupling an ultrasonic device to a body of a patient may comprise measuring a parameter of one or more of the ultrasonic device and the body, estimating a coupling state between the ultrasonic device and the body based on the measured parameter, and generating a user prompt corresponding to the coupling state between the ultrasonic device and the body. Such a system may be able to automatically guide a user towards achieving adequate ultrasonic coupling between an ultrasonic device and the body, thereby improving patient experience and benefit from the therapeutic or monitoring system.

In some variations, a method of noise reduction may be utilized which may allow decoupling of an ultrasound signal from a pressure signal measured by an IMD, thereby, allowing accurate recovery of physiological pressure data.

Generally, establishing a reliable wireless link between devices in a given wireless system may comprise one or more of the methods described herein, or any sub-set of the one or more methods described herein, or a combination of methods or sub-sets thereof. One or more methods described here, or steps therein, may be applied to a moving IMD, for example, an IMD that may move or rotate relative to a wireless device. One or more methods described here, or steps therein, may be applied to a plurality of IMDs that may be configured to exchange wireless signals with a wireless device.

### A. Wireless Device Positioning on the Body

A wireless device (e.g., a handheld device, a wearable device) may be used by inexperienced users such as patients (e.g., at home) for wirelessly powering and/or communicating with an implantable IMD (IMD). The user may not know the appropriate position of the wireless device on the body which may be required for establishing a robust wireless link with the IMD. This may lead to inadequate disease monitoring or therapy. Solutions are provided herein for foolproof positioning of a wireless device on the body.

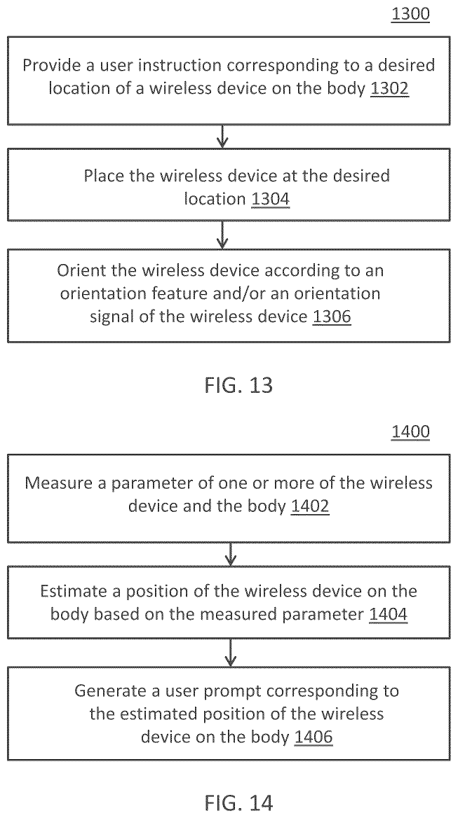

FIG. 13 is a flowchart that generally describes a variation of a method of positioning a wireless device on the body. The method (1300) may comprise the steps of providing a user instruction (e.g., displaying a picture of the desired location on the body, providing visual and/or audio instructions, and the like) corresponding to a desired location on the body (1302). Such an instruction may be provided by the wireless device to be positioned on the body and/or by a different device such as a phone, a computer, a tablet, and the like. Based on the user instruction, the user may place the wireless device at a location on the body (1304). Further, the wireless device may be oriented according to one or more of an orientation feature and an orientation signal of the wireless device (1306). An orientation feature may comprise one or more of a marking, a structure and a shape of the wireless device. In some variations, an orientation feature such as a marking may be configured to orient the wireless device with respect to a structure on the body. For example, the wireless device may be oriented on the chest such that the marking is close to or directed towards the left arm. An orientation signal may comprise a signal from one or more of an orientation sensor, an accelerometer, a gyroscope, a position sensor, combinations thereof, and the like. The orientation signal may indicate to the user if the wireless device is oriented adequately on the body or not, and may be configured to guide the user for proper positioning and orientation.

FIG. 14 is a flowchart that generally describes a variation of another method of positioning a wireless device on the body. The method may comprise the steps of measuring a parameter of one or more of the wireless device and the body (1402), estimating a position of the wireless device on the body based on the measured parameter (1404), and generating a user prompt corresponding to the estimated position of the wireless device on the body (1406). In some variations, the parameter may comprise one or more of a heart sound, a lung sound, a breathing sound, a wireless signal coming from an implantable IMD, a wireless reflection signal, a wireless backscatter signal, combinations thereof, and the like. For example, the wireless reflection signal and the wireless backscatter signal may be generated by the IMD and/or one or more tissue structures (e.g., ribs, lungs) upon transmission of an interrogation signal by the wireless device into tissue. In some variations, the wireless signals may be ultrasonic signals. Measurement of such one or more parameters and/or processing of the measured parameters by a processor of the wireless device, may indicate a proximity of the wireless device to a desired location on the chest.

In some variations, the user prompt may comprise one or more of a notification about the estimated position of the wireless device on the body, whether the positioning of the wireless device is adequate or not, and a recommendation comprising one or more of repositioning the wireless device on the body, re-orienting the wireless device, and contacting a health care professional. Repositioning the wireless device may comprise one or more of moving, adjusting and rotating the wireless device (e.g., its position, angle, rotation, tilt, direction, and the like). In some variations, mechanical and/or electrical parameters of the wireless device may also be adjusted in response to the user prompt. For example, adjusting electrical parameters may comprise recharging a battery of the wireless device.

Any permutations or combinations of the methods described herein, or parts thereof, may be used for ensuring adequate positioning of a wireless device on the body.

### B. Ultrasonic Coupling to Body

Adequate ultrasonic coupling between an ultrasonic device (e.g., wireless device) and the body of a patient is needed for wireless powering and communication with an implantable medical device using ultrasound. An air gap between the wireless device and body tissue is undesirable since it may disrupt an ultrasonic link between the wireless device and an implantable IMD (IMD) due to acoustic impedance mismatch. Avoiding such air gaps may be challenging if an inexperienced person such as a patient operates the ultrasonic device (e.g., at home), and/or if the ultrasonic device is being used for a prolonged duration. New methods of coupling an ultrasonic device to a body of a patient may be desirable.

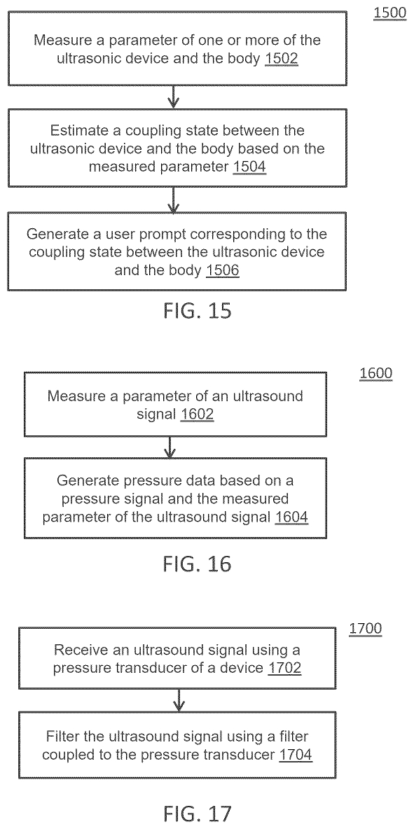

FIG. 15 is a flowchart that generally describes a variation of a method of coupling an ultrasonic device to a body of a patient. The method (1500) may comprise measuring a parameter of one or more of the ultrasonic device and the body (1502), estimating a coupling state between the ultrasonic device and the body based on the measured parameter (1504), and generating a user prompt corresponding to the estimated coupling state (1506). In some variations, the method may be repeated periodically and a user prompt may be provided until an adequate ultrasonic coupling is achieved.

The ultrasonic device may comprise one or more ultrasound transducers. In some variations, the parameter may comprise one or more of an electrical impedance of an ultrasound transducer of the ultrasonic device, reflection coefficient (e.g., an S11 parameter) of an ultrasound transducer of the ultrasonic device, a heart sound, an ultrasonic signal coming from an implantable medical device, an ultrasonic reflection signal, pressure, force, touch, capacitance, electrical impedance of tissue, heat, and temperature. In some variations, the ultrasonic device may comprise a processor and one or more sensors or transducers for measuring such one or more parameters.

In some variations, the electrical impedance of an ultrasound transducer, which may be affected by its surrounding medium due to acoustic loading, may be different with or without the presence of an air gap between the ultrasonic device and body tissue. In some variations, the ultrasonic device may comprise a processor configured to measure a reflection, a reflection coefficient and/or an S₁₁ parameter to assess coupling to body tissue. The reflection may be significant if an air gap is present. Whereas if an air gap is not present, an ultrasonic signal transmitted by the ultrasonic device may sufficiently penetrate through tissue, resulting in a small reflection. Such a reflection or an associated parameter (e.g., S₁₁) may be measured at one or more frequencies. In some variations, a processor of the ultrasonic device may process the measured reflection coefficient or S₁₁ and compare it to a threshold to determine whether the coupling is sufficient or not. For example, a magnitude of S₁₁ below a threshold may indicate adequate ultrasonic coupling of the wireless device to tissue.

In some variations, the ultrasonic device may comprise one or more of a pressure sensor, a force sensor, a touch sensor, combinations thereof, and the like. For example, a pressure or force sensor may be configured to detect if an ultrasonic device is sufficiently pressed against skin or tissue. As another example, a touch sensor (e.g., capacitive, resistive, surface acoustic wave, infrared, heat-based, and the like) may be configured to detect contact with skin or tissue. In some variations, the ultrasonic device may comprise one or more of an audio sensor or a pressure sensor to detect heart sounds (e.g., auscultation), a thermal or temperature sensor to detect heat or temperature associated with the body, combinations thereof, and the like, to assess coupling of the ultrasonic device to tissue.

In some variations, the ultrasonic device may be configured to determine whether there is sufficient coupling to tissue based on whether an uplink signal (e.g., a feedback signal) is received from an implantable device and/or based on measuring a strength of one or more uplink signals received by the ultrasonic device. In some variations, an implantable device may be configured to periodically transmit uplink signals such as a beacon signal at one or more beacon frequencies (e.g., about 100 Hz or lower). For example, if an ultrasonic device, when placed on the body and turned on, does not receive a beacon signal, or receives a low strength of a beacon signal compared to a threshold, for a duration greater than or equal to an expected time period between two consecutive beacons, it may be determined that the coupling between the ultrasonic device and tissue is inadequate and/or that the ultrasonic device is not positioned at the right location on the body.

An implantable device comprising a power source, such as a battery, may have sufficient energy to periodically transmit such beacon signals. In some variations, the beacon signal may comprise one or more of an ultrasonic pulse, an RF pulse, and the like. As an example, the beacon signal may be an ultrasonic pulse with a carrier frequency between about 0.1 MHz and 20 MHz, a pulse width of between about 0.05 μs to about 100 ms. In some variations, the beacon signal may encode information such as an ID code, an energy state of the implantable device (e.g., battery voltage), combinations thereof, and the like.

In some variations, the ultrasonic device may be configured to transmit an interrogation signal and to receive a corresponding feedback signal generated by an implantable device upon receiving the interrogation signal. The ultrasonic device may be further configured to estimate the coupling state based on measuring a strength of the received feedback signal, or based on whether a feedback signal is received or not.

In some variations, estimating a coupling state between the ultrasonic device and the body may comprise estimating one or more of the adequacy and degree of coupling between the ultrasonic device and the body. As an example, a processor of the ultrasonic device may be configured to compare a measured parameter such as S11 to a predetermined threshold to assess whether the coupling is adequate or not. In some variations, one or more measured parameters may be compared to a plurality of thresholds (e.g., by performing digitization) to assess a degree or extent of ultrasonic coupling. In some variations, an ultrasonic device may comprise a plurality of ultrasound transducers (e.g., array), where measurement of a parameter of the ultrasound transducers may be configured to assess which part or side of the ultrasonic device is adequately or inadequately coupled to body tissue.

In some variations, generating a user prompt corresponding to the coupling state between the ultrasonic device and the body may comprise providing to a user one or more of a notification about the coupling state and a recommendation comprising one or more of adjusting the ultrasonic device, repositioning the ultrasonic device against the body, applying an ultrasonic coupling agent (e.g., ultrasonic gel, silicone, a dry couplant, and the like), adding an acoustic matching layer, adjusting a fastener of the ultrasonic device to the body, and contacting a health care professional. Adjusting the ultrasonic device may comprise one or more of holding the ultrasonic device against skin, pushing or pressing the ultrasonic device towards tissue or skin, moving and/or rotating the ultrasonic device, cleaning a surface of the ultrasonic device and/or skin, removing hair between the ultrasonic device and skin, combinations thereof, and the like.

In some variations, an ultrasonic device may be secured or fastened (e.g., tightly) to the body to ensure good ultrasonic coupling to tissue using a fastener. For example, a fastener may comprise one or more of a belt, a strap, a sleeve, an adhesive, a clip or a pin (e.g., like a clip used for elastic bandages), a wearable structure, combinations thereof, and the like.

In some variations, the ultrasonic device may be located at one or more locations including, but not limited to, outside the body (e.g., on a wearable device, a handheld device, a probe connected to a measurement setup, a device placed or fastened to body, and the like), permanently implanted inside the body (e.g., implanted under the skin, along the outer wall of an organ), temporarily implanted inside the body (e.g., located on a catheter or a probe inserted through a blood vessel, esophagus or the chest wall, used during surgery or procedure), combinations thereof, and the like.

An example method is described here. In some variations, when placed on a patient's body and turned on, an ultrasonic device may be configured to first measure a parameter of one or more of its ultrasound transducers to estimate a coupling state and generate a user prompt to direct the user to adjust the ultrasonic device until adequate coupling is achieved. The ultrasonic device may then be configured to wait for a predetermined time duration to receive one or more beacon signals from an implantable device to assess its positioning on the body. In some variations, if the ultrasonic device is unable to receive any beacon signals from the implantable device, it may be configured to additionally measure a parameter such as heart sound, to detect proximity of the ultrasonic device to the patient's heart. Based on such measurements, a user prompt may be generated to guide the user for repositioning or moving the ultrasonic device in a desired direction. In some variations, if the ultrasonic device detects that it is placed at the right location on a patient's body and ultrasonic coupling to tissue is adequate, but is unable to receive any uplink signals from the implantable device, the user may be notified to consult a health care professional.

### C. Noise Reduction

An implantable IMD (IMD) may comprise one or more ultrasound transducers for exchanging one or more of wireless power and data with a wireless device (e.g., an external wireless device). The IMD may further comprise one or more pressure transducers for sensing pressure (e.g., blood pressure). Ultrasonic waves may be incident on such an IMD due to one or more of wireless power/data transmitted by the wireless device to the IMD, ultrasonic reflections in tissue, and one or more procedures such as ultrasonic imaging (e.g., transthoracic echocardiography or TTE). Since ultrasonic signals comprise of pressure waves, they may couple to a pressure transducer of an IMD (i.e., interfere with the pressure signal measured by the pressure transducer), and may corrupt the pressure data, resulting in an erroneous pressure value that may not accurately represent patient state. Solutions are presented herein to mitigate this challenge.

Methods of noise reduction (or interference mitigation) are presented for detecting and/or mitigating an interference of an ultrasound signal with a pressure signal. FIG. 16 is a flowchart that generally describes a variation of a method of noise reduction. The method (1600) may comprise measuring a parameter of an ultrasound signal (1602) received by one or more of an ultrasound transducer, pressure transducer, flow sensor, force sensor and MEMS device, and generating pressure data based on a pressure signal measured by the pressure transducer and the measured parameter of the ultrasound signal (1604). In some variations, generating the pressure data may comprise decoupling the ultrasound signal from the pressure signal. In some variations, decoupling the ultrasound signal from the pressure signal may comprise one or more of averaging, digital signal processing and analog signal processing.

In some variations, an IMD may be configured to measure a parameter of an ultrasound signal received by a pressure transducer. For example, the IMD may be configured to detect the presence and/or strength of ultrasound signals by taking a plurality of pressure samples per desired pressure datapoint. For example, if pressure datapoints are desired at a sampling rate of 100 Hz, instead of sampling pressure only once per 10 ms, an IMD may be configured to sample pressure more than once over an observation time (e.g., greater than 1 μs) at a high sampling rate (e.g., greater than 1 MHz). The plurality of pressure samples may be digitized (e.g., using an ADC) and processed using a processor of the IMD. Processing may happen in real time or post-processing may be performed after storing the samples in a memory of the IMD.

In some variations, after processing the plurality of pressure samples, the IMD's processor may determine if an ultrasonic signal may have coupled to the pressure transducer. One example of such processing is presented herein. A processor of the IMD may determine a variation in the values of the plurality of pressure samples per datapoint. For example, the processor may process values corresponding to the plurality of pressure samples per datapoint and determine one or more of a minimum value, a maximum value, a mean value, a difference between the maximum and minimum values, a variance, a standard deviation, a percentage change, combinations thereof, and the like. In some variations, if such a variation is found to be above a certain threshold (e.g., variation greater than ±1%, or a variation greater than ±10%, and the like), then it may indicate that an ultrasonic signal may have coupled to the pressure transducer. This may be because physiological or body pressure (e.g., blood pressure) may not be expected to change significantly within a short duration of time. For example, 5 pressure samples taken within a total duration of about 1 ms, may not vary by more than ±10% relative to each other, unless an ultrasonic signal may have coupled to the pressure transducer. Thus, a large change or variation in pressure samples taken within a short duration of time may be an indicator or a signature of ultrasonic coupling to the IMD's pressure transducer.

In some variations, an IMD may be configured to measure a parameter of an ultrasound signal received by an ultrasound transducer. For example, the IMD may be configured to detect the presence and/or strength of ultrasound signals by monitoring a voltage of its one or more ultrasound transducers, or a voltage generated by a circuit (e.g., a power circuit, an AC-DC converter circuit) connected to the one or more ultrasound transducers. For example, presence of a non-zero AC voltage, or an AC voltage above a certain threshold, at the terminals of an ultrasound transducer of the IMD may indicate a presence of incident ultrasound signals.

In some variations, generating the pressure data may comprise identifying one or more pressure samples of the pressure data comprising the measured parameter of the ultrasound signal, and rejecting or flagging the identified one or more pressure samples. For example, after an IMD detects that an ultrasonic signal may have coupled to a pressure signal for one or more desired pressure data points, it may ignore the samples and may instead write an error code (e.g., all 1's or all 0's, and the like) to its memory corresponding to that pressure data point. When the pressure data is transferred to a wireless device (e.g., via uplink data transfer), the wireless device may identify such an error code as being due to ultrasonic coupling to the pressure transducer or any other errors. In some variations, the wireless device may use any processing techniques such as extrapolation of error-free data to estimate pressure values that were corrupted due to ultrasonic coupling to the IMD's pressure transducer. In some variations, after an IMD detects that an ultrasonic signal may have coupled to the pressure transducer, the IMD's processor may process the plurality of pressure samples to decouple the effect of ultrasonic coupling (e.g., identify signatures of an ultrasound signal of about MHz frequency), estimate the actual physiological pressure for the datapoint, and store such an estimated pressure value to its memory.

In some variations, if a coupling of an ultrasound signal to an IMD's pressure transducer is detected, the IMD may be configured to measure a pressure signal after a time delay (e.g., once or periodically). In some variations, the time delay may be predetermined. For example, such a predetermined time delay may allow sufficient time for ultrasound signals, reflections and/or echoes to dissipate. In some variations, the time delay may be based on dissipation of the ultrasound signal. For example, the IMD may be configured to monitor a strength of the ultrasound signal and sample pressure after the strength is below a predetermined threshold.

In some variations, if no ultrasonic coupling to the pressure transducer is detected, the IMD's processor may compute an average pressure value of the one or more pressure samples taken per datapoint, and use such an average pressure value as the desired pressure datapoint (e.g., store such an average value in the memory). An advantage of taking a plurality of pressure samples per datapoint and computing an average value of such pressure samples for the datapoint is that it may help with reducing noise constraints on the design of an analog front end (AFE) circuit connected to the pressure transducer, lowering the value of the input sampling capacitance and/or the ADC capacitance (e.g., C_{DAC} in a SAR ADC), thereby, allowing faster settling of the pressure sample. Due to these advantages, the AFE and the pressure transducer may need to be powered, or connected to the power supply, for a shorter time duration per pressure sample, which may be beneficial in reducing the energy consumption of the IMD per pressure sample.

In some variations, the step of generating the pressure data is performed by a processor of a first device (e.g., IMD) comprising the ultrasound transducer and the pressure transducer. In some variations, the step of generating the pressure data is performed by a processor of a second device (e.g., wireless device) which may be in wireless communication with the first device (e.g., IMD). In some variations, an IMD may be configured to save all pressure values corresponding to the plurality of pressure samples, taken per datapoint, in its memory, and transfer all such values to the wireless device via uplink data transfer.

FIG. 17 is a flowchart that generally describes a variation of another method of noise reduction. The method (1700) may comprise the steps of receiving an ultrasound signal using a pressure transducer of a device such as an IMD (1702), and filtering the ultrasound signal using a filter coupled to the pressure transducer (1704). For example, an ultrasound signal at about 1 MHz frequency may result in fluctuations in the voltage of a pressure transducer at or close to about 1 MHz. On the other hand, desired pressure signals in the body may not have such a high frequency, and for instance, may have useful frequency content only up to about 200 Hz.

In some variations, filtering may comprise one or more of analog filtering, digital filtering, analog post-processing, digital post-processing and filtering using one or more of a filter, an amplifier, a processor, an integrator, an averager and a boxcar sampler. For example, an IMD may comprise a low-pass filter connected to a pressure transducer, either directly to the pressure transducer's output terminals, or after sufficiently amplifying the pressure transducer's signal using a pre-amplifier. The output of the filter may be connected to an ADC for digitizing the pressure sample. In some variations, a cut-off frequency and roll off of such a low-pass filter may be set such that the filter sufficiently attenuates undesired signals, such as the ultrasound signal in this case, based on frequency. In the example discussed here, in some variations, a cut-off frequency of about 500 kHz may be used as an example. Using a high cut-off frequency for the low-pass filter (e.g., that is still sufficiently low to attenuate high frequency ultrasound signals), and/or a high roll off, may allow fast settling of the pressure samples.

### D. Heart Rate Estimation

In some applications, an IMD (IMD) may be configured to sense blood pressure (e.g., in one of the heart chambers, a blood vessel, etc.). Sensing of blood pressure (BP) may be important for the diagnosis and/or monitoring of cardiovascular diseases including but not limited to one or more of heart failure, congestive heart failure, arrhythmia, combinations thereof, and the like. In some variations, measurement of a heart rate (HR), alternatively or in addition to BP, may be important. For example, a physician may assess the HR of a patient in conjunction with BP, and this may be useful for one or more of accurate monitoring of the progression of heart failure, diagnosis and/or monitoring of arrhythmia, diagnosis of a condition or an event, adjustment of a patient's therapy or medication, combinations thereof, and the like. In some variations, an IMD may measure BP (and may transmit measured BP data in real time, or at a later time, to a wireless device), while a wireless device (or any wireless device such as a smart watch) may measure HR simultaneously, thereby, allowing a physician to assess the HR values at the time the BP measurements were taken. In some variations, an IMD may be configured to measure a HR (e.g., using electrodes similar to an ECG measurement), in addition to BP measurements, and transmit both BP and HR data (e.g., raw data such as waveforms, or after computing one or more HR values for one or more cardiac cycles) to the wireless device. In some applications, an IMD may need to have a miniature size such that it may not be desirable, or possible, to include electrodes on the IMD for measuring HR directly. In some applications, the IMD may be configured to measure BP continuously or at many time points per day, during which, it may not be desirable, or possible, to have a wireless device configured for measuring HR simultaneously. Solutions are provided herein for estimating a heart rate based on blood pressure measurements.

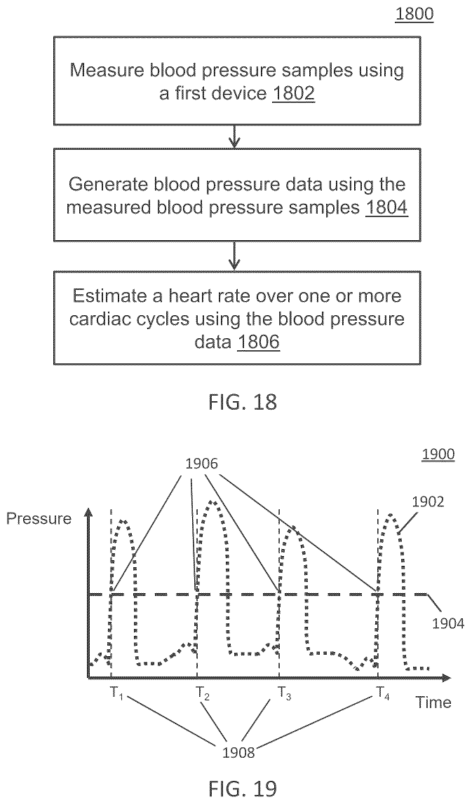

FIG. 18 is a flowchart that generally describes a variation of a method of estimating a heart rate. The method (1800) may comprise the steps of measuring blood pressure samples using a first device such as an IMD (1802), generating blood pressure data using the measured blood pressure samples (1804), and estimating a heart rate over one or more cardiac cycles using the blood pressure data (1806). In some variations, estimating the heart rate may be performed by a processor of the first device. In some variations, estimating the heart rate may be performed by a processor of a second device (e.g., a wireless device) that may be in wireless communication with the first device (e.g., an IMD). For example, the first device may wirelessly transmit blood pressure data (e.g., a BP waveform) to the second device in real time or at a later time after storing the waveform in a memory of the first device.

In some variations, estimating a heart rate may comprise comparing one or more of the blood pressure samples to a predetermined threshold, identifying two or more cross-over points where the blood pressure samples cross the predetermined threshold, and estimating a heart rate based on one or more elapsed times between the identified cross-over points. FIG. 19 illustrates a conceptual timing diagram (1900) corresponding to such a method. In some variations, an IMD may sample BP at a certain rate (e.g., about 100 Hz, about 1 kHz, and the like), and the IMD may have a timer or a clock that may, for instance, have a count or information about a time point corresponding to a sample. In some variations, the IMD's processor may be configured to digitize the sensed pressure (e.g., using an ADC) and/or to compare a sensed pressure value of a BP waveform (1902) to a threshold (1904). Upon comparison of one or more pressure samples to the threshold in real time, when a sensed pressure value may be found to cross the threshold (e.g., when the BP is rising, or when the BP is falling, in a given cardiac cycle), the IMD may store a value corresponding to the time at which this cross over may happen. Such a crossing of a threshold (1904) by a BP waveform (1902) may be termed as a cross-over point (1906), which is conceptually illustrated in FIG. 19. For example, an IMD may determine timer counts (1908) T₁, T₂, T₃, T₄ and the like, corresponding to such cross-over points (1906), as shown in FIG. 19. Such a timer count (1908) may be determined for more than one cardiac cycle (any number of cardiac cycles), and may be processed by a processor of the IMD to determine a HR, or an average HR over a certain time duration, HR variation, combinations thereof, and the like. For example, if the time corresponding to a first cross-over point is T₁ seconds, and the time corresponding to the next cross-over point is T₂ seconds, then an instantaneous HR (i.e., HR for one cardiac cycle) may be estimated as 1/(T₂−T₁) Hz or 60/(T₂−T₁) beats per minute. Similarly, times corresponding to a plurality of cross-over points (1906) may be determined and processed to estimate an average HR. An advantage of such a technique may be a reduced sensitivity of the estimated HR value to fluctuations in the BP waveform (e.g., changing values of peak BP in consecutive cycles, etc.), and/or to the sampling rate, since the measurement relies on the part of the BP waveform that has a large slope. Fluctuations, variations or shifts in the BP waveform may occur due to one or more reasons including, but not limited to, breathing, atmospheric pressure variations (if the measured BP is an absolute pressure), combinations thereof, and the like. As long as a part of the BP waveform with a large slope crosses the threshold level periodically (either while rising or while falling), it may be possible to determine the HR value using this technique with a reasonable accuracy.

Any method may be used for measuring the elapsed time (e.g., T₂−T₁) between consecutive cross-over points (see FIG. 19). In some variations, an IMD may have a running timer or a clock, and may determine a timer count corresponding to a cross-over point, as discussed above. In some variations, the IMD may comprise a time-to-digital converter or TDC circuit (e.g., comprising a ramp interpolator or a relaxation oscillator) to measure the time between two cross-over points. For example, charging of a known capacitor with a known current source may be started at one cross-over point (e.g., triggered by the output of a comparator that compares rising BP values to a threshold), and an increase in the voltage of the capacitor (due to the charging) may be measured at the next cross-over point, in order to estimate the elapsed time between the two cross-over points. It may be noted that techniques mentioned here are examples, and other variations of measuring the times corresponding to the cross-over points, or the elapsed time between two cross-over points, may be used.

In some variations, the threshold, as described above and shown in FIG. 19, may be adaptable and may be determined by the IMD in real time, or from past BP measurements. For example, the IMD may determine a mean BP value, or may compute an arithmetic mean of maximum and minimum BP values (i.e., (max+min)/2), or may compute a fraction of the peak pressure (e.g., 80%), from one or more cardiac cycles, and designate this value as a threshold for HR estimation in the next one or more cardiac cycles. In some variations, such an adaptable threshold may be useful if the BP waveform shifts significantly due to changes in atmospheric pressure, breathing, or any other reasons. In some variations, the threshold may be fixed to a pre-set value. For example, in some variations, a threshold of about 820 mmHg absolute pressure may be used for a pressure sensor in the LV, if the atmospheric pressure is known to be about 760 mmHg, and the like. In some variations, a wireless device may transmit a value of a threshold, or a value useful for determining the threshold (e.g., value of atmospheric pressure, and the like), to the IMD via downlink data. Optionally or additionally, before transmitting such a value to the IMD via downlink signals, the wireless device may measure and digitize the atmospheric pressure.

In some variations, estimating a heart rate may comprise identifying points of local maxima or minima in the blood pressure samples, and estimating a heart rate based on one or more elapsed times between two or more points of local maxima or minima. For example, a processor may be configured to detect a local maximum or minimum value of the BP in every cardiac cycle, save a timer count corresponding to the occurrence of the maximum or minimum BP values, and process the timer count values to estimate a HR or an average HR, and the like. In some variations, an IMD's processor may be configured to perform a running maximum or running minimum computation in real time during the process of measuring blood pressure samples. In some variations, a processor may be configured to pass the blood pressure data through a filter or a smoothing window in order to mitigate the effect of random or temporary fluctuations and/or noise in the sensed pressure samples.

In some variations, estimating a heart rate may comprise identifying points of maximum or minimum rate of change in the blood pressure samples, and estimating a heart rate based on one or more elapsed times between two or more points of maximum or minimum rate of change. For example, a processor may comprise a differentiator circuit to compute a rate of change of the blood pressure samples.

In some variations, estimating a heart rate is based on a frequency domain representation of the blood pressure samples. For example, a processor may be configured to compute a Fourier transform or a fast-Fourier transform (FFT) of the BP waveform to determine a frequency or time period of the BP waveform.

In some variations, it may not be desirable, or possible, to store a complete BP waveform in an IMD's memory due to reasons such as memory size limitations, and/or to process a complete BP waveform in an IMD's processor due to limitations in the energy and/or time required for complex computations. In such variations, an IMD may be configured to process sensed BP values in real time and determine a heart rate using one or more of the methods described herein.

In some variations, in general (i.e., applicable to any method described herein, and not necessarily only for the method of determining HR from BP measurements), an IMD may ignore certain pressure samples during processing and before computing a maximum, minimum, peak, and/or mean pressure values. For example, such ignored pressure samples may comprise anomalous pressure values due to one or more of undesired coupling of an ultrasound signal to a pressure transducer, coughing and/or sneezing of a patient, sudden movement of a patient, sudden movement of tissue (e.g., heart wall, blood vessel) and/or the IMD, any temporary anomalies or irregularities, combinations thereof, and the like. In some variations, one or more anomalous pressure values may be identified by comparing a given pressure sample to one or more previous pressure samples (e.g., an extrapolated version of one or more previous pressure samples), predetermined thresholds, thresholds determined in real time based on previous pressure samples, combinations thereof, and the like.

Any permutations or combinations of the methods described herein, or parts thereof, may be used for estimating a heart rate. One or more of the methods described herein may be configured to estimate other parameters or events related to blood pressure samples, similar to the estimation of heart rate.

The specific examples and descriptions herein are exemplary in nature and variations may be developed by those skilled in the art based on the material taught herein without departing from the scope of the present invention, which is limited only by the attached claims.

1.-58. (canceled)

59. A method of exchanging wireless signals, comprising:
transmitting an interrogation signal to a first device using a first sub-array of a second device;
receiving a feedback signal from the first device using a second sub-array of the second device;
selecting one or more transducer configurations of the second device based on the feedback signal; and
exchanging one or more wireless signals with the first device using the one or more transducer configurations of the second device during one or more intervals, wherein the wireless signals comprise one or more of a power signal, data signal, interrogation signal, feedback signal, downlink signal and uplink signal.

60. The method of claim 59, further comprising transmitting the feedback signal from the first device in response to one or more wireless signals received by the first device.

61. The method of claim 59, further comprising detecting one or more of a falling edge of one or more wireless signals and a code corresponding to one or more wireless signals received by the first device.

62. The method of claim 59, wherein selecting one or more transducer configurations of the second device comprises one or more of determining one or more of a frequency, a delay, a phase, an amplitude and a gain of the selected one or more transducer elements based at least in part on one or more of a delay, phase, arrival time, time of flight, amplitude, frequency, and encoded data of the feedback signal.

63. The method of claim 59, further comprising determining to transmit one or more of a power signal, interrogation signal, data signal and a downlink signal to the first device in response to the received feedback signal.

64. (canceled)

65. The method of claim 59, wherein a transducer configuration corresponding to a subsequent interval is selected based on one or more previously received feedback signals during one or more previous intervals.

66. The method of claim 59, wherein a duration of at least one interval of the one or more intervals is determined by the first device.

67. The method of claim 59, wherein a duration of at least one interval of the one or more intervals is determined by the second device.

68. The method of claim 59, wherein the first device is configured to periodically transmit the feedback signals during one or more of the intervals.

69.-73. (canceled)

74. The method of claim 59, wherein selecting one or more of the transducer configurations of the second device comprises:
estimating a strength of the feedback signal received by the second sub-array of the second device; and
exchanging one or more of the wireless power signal and data signal using the one or more transducer configurations based on the estimated strength of the received feedback signal.

75. (canceled)

76. The method of claim 59, wherein the feedback signal comprises a first feedback signal, and the method further comprising:
powering the first device by transmitting a first power signal during a first power interval; and
receiving a second feedback signal from the first device after the first power interval.

77.-80. (canceled)

81. The method of claim 59, further comprising selecting the transducer configuration based on a location of the first device.

82.-83. (canceled)

84. The method of claim 59, wherein a duration of the interval is predetermined.

85.-88. (canceled)

89. The method of claim 59, further comprising generating a location notification corresponding to a spatial adjustment of the second device.

90.-93. (canceled)

94. The method of claim 89, wherein generating the location notification is based on the feedback signal.

95. The method of claim 59, comprising generating a power notification comprising a power state of one or more of the first device and the second device.

96. The method of claim 59, comprising generating a communication notification corresponding to one or more of data received from the first device, physiological parameter data, and parameter data of one or more of the first device and the second device.

97.-159. (canceled)
